(19) United States
(12) Patent Application Publication
(10) Pub. No.: US 2006/0240048 A1
(43) Pub. Date: Oct. 26, 2006

(54) Pyridine derivatives as cb2 receptor modulators

(76) Inventors: Andrew John Eatherton, Welwyn, Hertfordshire (GB); Gerard Martin Paul Giblin, Hertfordshire (GB); Richard Howard Green, Hertfordshire (GB); Jennifer Margaret Doughty, Gorham, ME (US); Karamjit Singh Jandu, Hertfordshire (GB); William Leonard Mitchell, Hertfordshire (GB); Alan Naylor, Essex (GB); Giovanni Palombi, Milan (IT); Derek Anthony Rawlings, Hertfordshire (GB); Brian Peter Slingsby, Hertfordshire (GB); Andrew Richard Whittington, Hertfordshire (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/528,613

(22) PCT Filed: Sep. 25, 2003
(86) PCT No.: PCT/EP03/10930

§ 371 Date: Feb. 28, 2006

(30) Foreign Application Priority Data
Sep. 27, 2002 (GB) 0222493.9

(51) Int. Cl. A61K 47/00

(52) U.S. Cl. 424/278.1

(57) ABSTRACT

The present invention relates to novel pyridine derivatives, pharmaceutical compositions containing these compounds and their use in the treatment of diseases, particularly pain, which diseases are caused directly or indirectly by an increase or decrease in activity of the cannabinoid receptor.

The present invention relates to novel pyridine derivatives, pharmaceutical compositions containing these compounds and their use in the treatment of diseases, particularly pain, which diseases are caused directly or indirectly by an increase or decrease in activity of the cannabinoid receptor.

Cannabinoids are a specific class of psychoactive compounds present in Indian cannabis (Cannabis sativa), including about sixty different molecules, the most representative being cannabinol, cannabidiol and several isomers of tetrahydrocannabinol. Knowledge of the therapeutic activity of cannabis dates back to the ancient dynasties of China, where, 5,000 years ago, cannabis was used for the treatment of asthma, migraine and some gynaecological disorders. These uses later became so established that, around 1850, cannabis extracts were included in the US Pharmacopaeia and remained there until 1947.

Cannabinoids are known to cause different effects on various systems and/or organs, the most important being on the central nervous system and on the cardiovascular system. These effects include alterations in memory and cognition, euphoria, and sedation. Cannabinoids also increase heart rate and vary systemic arterial pressure. Peripheral effects related to bronchial constriction, immunomodulation, and inflammation have also been observed. The capability of cannabinoids to reduce intraocular pressure and to affect respiratory and endocrine systems is also well documented. See e.g. L. E. Hollister, Health Aspects of Cannabis, Pharmacological Reviews, Vol. 38, pp. 1-20, (1986). More recently, it was found that cannabinoids suppress the cellular and humoral immune responses and exhibit antiinflammatory properties. Wirth et al., Antiinflammatory Properties of Cannabichrome, Life Science, Vol. 26, pp. 1991-1995, (1980).

In spite of the foregoing benefits, the therapeutic use of cannabis is controversial, both due to its relevant psychoactive effects (causing dependence and addiction), and due to manifold side effects that have not yet been completely clarified. Although work in this field has been ongoing since the 1940's, evidence indicating that the peripheral effects of cannabinoids are directly mediated, and not secondary to a CNS effect, has been limited by the lack of receptor characterization, the lack of information concerning an endogenous cannabinoid ligand and, until recently, the lack of receptor subtype selective compounds.

The first cannabinoid receptor was found to be mainly located in the brain, in neural cell lines, and, only to a lesser extent, at the peripheral level. In view of its location, it was called the central receptor (“CB1”). See Matsuda et al., “Structure of a Cannabinoid Receptor and Functional Expression of the Cloned cDNA,” Nature, Vol. 346, pp. 561-564 (1990. The second cannabinoid receptor (“CB2”) was identified in the spleen, and was assumed to modulate the non psychoactive effects of the cannabinoids. See Munro et el., “Molecular Characterization of a Peripheral Receptor for Cannabinoids,” Nature, Vol. 365, pp. 61-65 (1993).

Recently, some compounds have been prepared which are capable of acting as agonists on both the cannabinoid receptors. For example, use of derivatives of dihydroxypyrrole-(1,2,3-d,e)-1,4-benzoxazine in the treatment of glaucoma and the use of derivatives of 1,5-diphenyl-pyrazole as immunomodulators or psychotropic agents in the treatment of various neuropathologies, migraine, epilepsy, glaucoma, etc are known. See U.S. Pat. No. 5,112,820 and EP 576357, respectively. However, because these compounds are active on both the CB1 and CB2 receptor, they can lead to serious psychoactive effects.

The foregoing indications and the preferential localization of the CB2 receptor in the immune system confirms a specific role of CB2 in modulating the immune and antiinflammatory response to stimuli of different sources.

The total size of the patient population suffering from pain is vast (almost 300 million), dominated by those suffering from back pain, osteo-arthritic pain and post-operative pain. Neuropathic pain (associated with neuronal lesions such as those induced by diabetes, HIV, herpes infection, or stroke) occurs with lower, but still substantial prevalence, as does cancer pain.

The pathogenic mechanisms that give rise to pain symptoms can be grouped into two main categories:
those that are components of inflammatory tissue responses (Inflammatory Pain);
those that result from a neuronal lesion of some form (Neuropathic Pain).

Chronic inflammatory pain consists predominantly of osteoarthritis, chronic low back pain and rheumatoid arthritis. The pain results from acute and on-going injury and/or inflammation. There may be both spontaneous and provoked pain.

There is an underlying pathological hypersensitivity as a result of physiological hyperexcitability and the release of inflammatory mediators which further potentiate this hyperexcitability. CB2 receptors are expressed on inflammatory cells (T cells, B cells, macrophages, mast cells) and mediate immune suppression through inhibition of cellular interaction/inflammatory mediator release. CB2 receptors may also be expressed on sensory nerve terminals and therefore directly inhibit hyperalgesia.

The role of CB2 in immunomodulation, inflammation, osteoporosis, cardiovascular, renal and other disease conditions is now being examined. In light of the fact that cannabinoids act on receptors capable of modulating different functional effects, and in view of the low homology between CB2 and CB1, the importance of developing a class of drugs selective for the specific receptor sub-type is evident. The natural or synthetic cannabinoids currently available do not fulfil this function because they are active on both receptors.

Based on the foregoing, there is a need for compounds which are capable of selectively modulating the receptor for cannabinoids and, therefore, the pathologies associated with such receptors. Thus, CB2 modulators offer a unique approach toward the pharmacotherapy of immune disorders, inflammation, osteoporosis, renal ischemia and other pathophysiological conditions.

The present invention provides novel pyridine derivatives of formula (I) and pharmaceutically acceptable derivatives thereof, pharmaceutical compositions containing these compounds or derivatives, and their use as CB2 receptor modulators, which are useful in the treatment of a variety of disorders.

The present invention further comprises a method for treating disease mediated by CB2 receptors in an animal, including humans, which comprises administering to an animal in need thereof an effective amount of a compound of formula (I) or a pharmaceutically acceptable derivative thereof.

The invention provides compounds of formula (I):

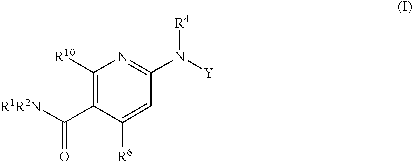

wherein:

Y is phenyl, unsubstituted or substituted with one, two or three substituents;

R¹ is selected from hydrogen, C₁₋₆ alkyl, C₃₋₆ cycloalkyl, or halosubstitutedC₁₋₆ alkyl;

R² is (CH₂)ₘR³ where m is 0 or 1;

or R¹ and R² together with N to which they are attached form an optionally substituted 4 to 8-membered non-aromatic heterocyclyl ring;

R³ is a 4- to 8-membered non-aromatic heterocyclyl group, a C₃₋₈ cycloalkyl group, a straight or branched C₁₋₁₀ alkyl, a C₂₋₁₀alkenyl, a C₃₋₈cycloalkenyl, a C₂₋₁₀alkynyl, or a C₃₋₈cycloalkynyl any of which can be unsubtituted or substituted or R⁵;

R⁴ is selected from hydrogen, C₁₋₆ alkyl, C₃₋₆ cycloalkyl, or halosubstitutedC₁₋₆ alkyl, COCH₃, or SO₂Me;

R⁵ is

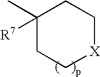

wherein

p is 0, 1 or 2, and X is CH₂, O, or S;

R⁶ is a substituted or unsubstituted (C₁₋₆)alkyl or chloro and R¹⁰ is hydrogen or R¹⁰ is a substituted or unsubstituted (C₁₋₆)alkyl or chloro and R⁶ is hydrogen;

R⁷ is OH, C₁-alkoxy, NR^{8a}R^{8b}, NHCOR⁹, NHSO₂R⁹ or SOqR⁹;

R^{8a} is H or C₁₋₆alkyl;

R^{8b} is H or C₁₋₆alkyl;

R⁹ is C₁₋₆-alkyl;

q is 0, 1 or 2;

and pharmaceutically acceptable derivatives thereof.

In one particular embodiment Y is a substituted phenyl. In one particular embodiment Y is substituted by 1 or 2 substituents. If mono-substituted, in one particular embodiment, the substituents is in the 3 position.

When Y is substituted, the substituent or substituents are preferably selected from: C₁₋₆ alkyl, halosubstitutedC₁₋₆ alkyl, C₁₋₆ alkoxy, a hydroxy group, a cyano group, halo, a C₁₋₆alkylsulfonyl group, —CONH₂, —NHCOCH₃ or —COOH. Further the substituent or substituents can be selected from halosubstitutedC₁₋₆ alkoxy, SO₂NR^{8a}R^{8b} wherein R^{8a} and R^{8b} are as defined above or C₁₋₆ alkynyl. In one particular embodiment Y is substituted by halo, cyano, methoxy, trifluoromethoxy or methyl.

A further aspect of the invention are compounds of formula (Ia):

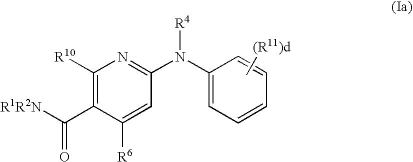

wherein:

R¹ is selected from hydrogen, C₁₋₆ alkyl, C₃₋₆ cycloalkyl, or halosubstitutedC₁₋₆ alkyl;

R² is (CH₂)ₘR³ where m is 0 or 1;

or R¹ and R² together with N to which they are attached form a non-aromatic heterocyclyl ring selected from azetidinyl, pyrrolidinyl, morpholinyl, piperazinyl, piperidinyl, tetrahydropyridinyl, azapine, oxapine, azacyclooctanyl, azaoxacyclooctanyl and azathiacyclooctanyl, any of which can be unsubstituted or substituted with 1, 2 or 3 substituents selected from; C₁₋₆ alkyl, C₁₋₆ alkoxy, hydroxy, cyano, halo, sulfonyl, methylsulfonyl, NR^{8a}R^{8b}, CH₂phenyl, NHCOCH₃, (═O), CONHCH₃ and NHSO₂CH₃;

R³ is 2- or 3-azetidinyl, oxetanyl, thioxetanyl, thioxetanyl-s-oxide, thioxetanyl-s,s-dioxide, dioxalanyl, pyrrolidinyl, tetrahydrofuranyl, tetrahydrothiophenyl, tetrahydrothiophenyl-s,s-dioxide, morpholinyl, piperidinyl, piperazinyl, tetrahydropyranyl, tetrahydrothiopyranyl, thiomorpholinyl, thiomorpholinyl-s,s-dioxide, tetrahydropyridinyl, dioxanyl, tetrahydro-thiopyran 1,1 dioxide, azapine, oxapine, azacyclooctanyl, azaoxacyclooctanyl, azathiacyclooctanyl, oxacylcooctanyl, thiacyclooctanyl, a C₃₋₈ cycloalkyl group, a straight or branched C₁₋₁₀ alkyl, a C₂₋₁₀alkenyl, a C₃₋₈cycloalkenyl, a C₂₋₁₀alkynyl, or a C₃₋₈ cycloalkynyl or R⁵; any of which can be unsubstituted or substituted with 1, 2 or 3 substituents selected from C₁₋₆ alkyl, C₁₋₆ alkoxy, hydroxy, cyano, halo, sulfonyl, methylsulfonyl, NR^{8a}R^{8b}, CH₂phenyl, NHCOCH₃, (═O), CONHCH₃ and NHSO₂CH₃;

R⁴ is selected from hydrogen, C₁₋₆ alkyl, C₃₋₆ cycloalkyl, or halosubstitutedC₁₋₆ alkyl, COCH_{3,} or SO₂Me;

R⁵ is

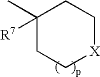

wherein

p is 0, 1 or 2, and X is CH_{2,} O or S;

R⁶ is a substituted or unsubstituted (C₁₋₆)alkyl or chloro and R¹⁰ is hydrogen or R¹⁰ is a substituted or unsubstituted (C₁₋₆)alkyl or chloro and R⁶ is hydrogen;

R⁷ is OH, C₁₋₆alkoxy, NR^{8a}R^{8b}, NHCOR⁹, NHSO₂R⁹ or SOqR⁹;

R^{8a} is H or C₁₋₆alkyl;

R^{8b} is H or C₁₋₆alkyl;

R⁹ is C₁₋₆alkyl;

R¹¹ is C₁₋₆ alkyl, halosubstitutedC₁₋₆ alkyl, C₁₋₆ alkoxy, hydroxy, cyano, halo, C₁₋₆alkylsulfonyl group, —CONH₂, —NHCOCH₃, —COOH, halosubstituted C₁₋₆ alkoxy SO₂NR^{8a}R^{8b} or C₁₋₄ alkynyl;

q is 0, 1 or 2;

d is 0, 1, 2, or 3;

and pharmaceutically acceptable derivatives thereof.

In one particular embodiment R¹ is hydrogen.

In one particular embodiment R⁴ is C₁₋₆ alkyl or hydrogen, more preferably methyl or hydrogen, even more preferably hydrogen.

In one particular embodiment X is CH₂ or O.

When R¹ and R² together with N to which they are attached form a 4- to 8-membered non-aromatic heterocyclyl ring which is substituted, or when R³ is substituted, they may be substituted with 1, 2 or 3 substituents preferably selected from: C₁₋₆ alkyl, C₁₋₆ alkoxy, a hydroxy group, a cyano group, halo or a sulfonyl group. Additionally the optional substituent(s) can be selected from methylsulfonyl, NR^{8a}R^{8b}, CH₂phenyl, NHCOCH₃, (═O), CONHCH₃ or NHSO₂CH₃ wherein R^{8a} and R^{8b} are as defined for formula (I).

When R⁶ or R¹⁰ are substituted alkyl groups, they can be substituted with 1, 2 or 3 substitutents selected from hydroxy, C₁₋₆-alkyoxy, cyano, halo, NR^{8a}R^{8b}, CONR^{8a}R^{8b}, SO₂NR^{8a}R^{8b}, NR^{8a}COR^{8b} or NR^{8a}SO₂R^{8b}, preferably hydroxy or fluorine.

In one particular embodiment R¹ and R² together with the N to which they are attached form an optionally substituted 5-or 6-membered non-aromatic heterocyclyl ring.

In one particular embodiment R⁶ is a substituted or unsubstituted (C₁₋₆)alkyl, chloro or CHxFn wherein n is 1, 2, or 3, x is 0, 1 or 2 and n and x add up to 3 and R¹⁰ is hydrogen or R¹⁰ is a substituted or unsubstituted (C₁₋₆)alkyl, chloro or CHxFn wherein n is 1, 2, or 3, x is 0, 1 or 2 and n and x add up to 3 and R⁶ is hydrogen

In one particular embodiment R⁶ is t-butyl, isopropyl or CHxFn, more preferably R⁶ is isopropyl or CHxFn even more preferably isopropyl or CF₃ and R¹⁰ is hydrogen or R¹⁰ is t-butyl, isopropyl or CHxFn, more preferably R¹⁰ is isopropyl or CHxFn, more preferably isopropyl or CF₃ and R⁶ is hydrogen.

In one particular embodiment R¹⁰ is hydrogen.

In one particular embodiment R⁷ is OH.

In one particular embodiment R⁵ is

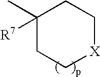

wherein p is 0, 1 or 2.

In one particular embodiment when R³ is an optionally substituted C₃₋₈cycloalkyl group or an optionally substituted 4- to 8-membered nonaromatic heterocyclyl, m is 1.

In one particular embodiment R³ is an optionally substituted C₃₋₆ cycloalkyl group or an optionally substituted 4- or 6-membered nonaromatic heterocyclyl.

In one particular embodiment when R¹ and R² taken together with the N to which they are attached form an optionally substituted heterocyclyl ring, the ring may be selected from pyrrolidinyl, morpholinyl, piperazinyl, piperidinyl and tetrahydropyridinyl.

In one particular embodiment when R³ is an optionally substituted non-aromatic heterocyclyl group selected from dioxalanyl, pyrrolidinyl, tetrahydrofuranyl, tetrahydrothiophenyl, tetrahydrothiophenyl-s,s-dioxide, morpholinyl, piperidinyl, piperazinyl, tetrahydropyranyl, tetrahydrothiopyranyl, dioxanyl, thiomorpholinyl, dioxanyl, thiomorpholinyl-s,s-dioxide and tetrahydropyridinyl.

A further aspect of the invention are compounds of formula (Ib):

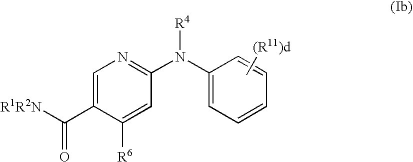

wherein:

R¹ is selected from hydrogen;

R² is (CH₂)ₘR³ where m is 0 or 1;

or R¹ and R² together with N to which they are attached form pyrrolidinyl, morpholinyl, piperazinyl, piperidinyl, tetrahydropyridinyl, any of which can be unsubstituted or substituted with 1, 2 or 3 substituents selected from C₁₋₆ alkyl, C₁₋₆ alkoxy, hydroxy, cyano, halo, sulfonyl, methylsulfonyl, NR^{8a}R^{8b}, CH₂phenyl, NHCOCH₃, (═O), CONHCH₃ and NHSO₂CH₃;

R³ is dioxalanyl, pyrrolidinyl, tetrahydrofuranyl, tetrahydrothiophenyl, tetrahydrothiophenyl-s,s-dioxide, morpholinyl, piperidinyl, piperazinyl, tetrahydropyranyl, tetrahydrothiopyranyl, thiomorpholinyl, thiomorpholinyl-s,s-dioxide, dioxanyl, tetrahydropyridinyl, a C₃₋₈ cycloalkyl group, a straight or branched C₁₋₁₀ alkyl; any of which can be unsubstituted or substituted with 1, 2 or 3 substituents selected from C₁₋₆ alkyl, C₁₋₆ alkoxy, hydroxy, cyano, halo, sulfonyl, methylsulfonyl, NR^{8a}R^{8b}, CH₂phenyl, NHCOCH₃, (═O), CONHCH₃ or NHSO₂CH₃; or R⁵.

R⁴ is selected from hydrogen, C₁₋₆ alkyl, C₃₋₆ cycloalkyl, or halosubstitutedC₁₋₆ alkyl, COCH₃, or SO₂Me;

R⁵ is

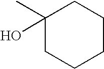

R⁶ is a substituted or unsubstituted (C₁₋₆)alkyl or chloro;

R^{8a} is H or C₁₋₆alkyl;

R^{8b} is H or C₁₋₆alkyl;

R¹¹ is C₁₋₆ alkyl, halosubstitutedC₁₋₆ alkyl, C₁₋₆ alkoxy, hydroxy, cyano, halo, C₁₋₆alkylsulfonyl group, —CONH₂, —NHCOCH₃, —COOH, halosubstituted C₁₋₆ alkoxy, SO₂NR^{8a}R^{8b} or C₁₋₆ alkynyl;

d is 0, 1, 2, or 3;

and pharmaceutically acceptable derivatives thereof.

Alternatively compounds of formula (I) can be selected from compounds of formula (Ic);

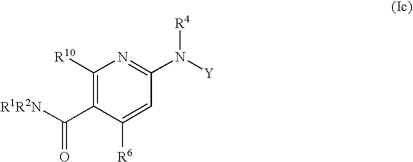

wherein:

Y is phenyl, optionally substituted with one, two or three substituents;

R¹ is selected from hydrogen, C₁₋₆ alkyl, C₃₋₆ cycloalkyl, or halosubstitutedC₁₋₆ alkyl;

R² is (CH₂)ₘR³ where m is 0 or 1;

or R¹ and R² together with N to which they are attached form an optionally substituted 5- or 6-membered non-aromatic heterocyclyl ring;

R³ is an optionally substituted 4- to 8-membered non-aromatic heterocyclyl group, an optionally substituted C₃₋₈ cycloalkyl group, an optionally substituted straight or branched C₁₋₁₀ alkyl or R⁵;

R⁴ is selected from hydrogen, C₁₋₆ alkyl, C₃₋₆ cycloalkyl, or halosubstitutedC₁₋₆ alkyl, COCH₃, or SO₂Me;

R⁵ is

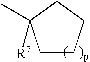

wherein

p is 0, 1 or 2;

R⁸ is (C₁₋₆)alkyl, chloro or CHxFn wherein n is 1, 2, or 3, x is 0, 1 or 2 and n and x add up to 3 and R¹⁰ is hydrogen or R¹⁰ is (C₁₋₆)alkyl, chloro or CHxFn wherein n is 1, 2, or 3, x is 0, 1 or 2 and n and x add up to 3 and R⁶ is hydrogen;

R⁷ is OH, C₁₋₆alkoxy, NR^{8a}R^{8b}, NHCOR⁹, NHSO₂R⁹, SOqR⁹;

R^{8a} is H or C₁₋₆alkyl;

R^{8b} is H or C₁₋₆alkyl;

R⁹ is C₁₋₆alkyl;

q is 0, 1 or 2;

and pharmaceutically acceptable derivatives thereof.

In one particular embodiment the compounds are selective for CB2 over CB1. Preferably the compounds are 100 fold selective i.e. compounds of formula (I) have an EC50 value at the cloned human cannabinoid CB2 receptor of at least 100 times the EC₅₀ values at the cloned human cannabinoid CB1 receptor or have less than 10% efficacy at the CB1 receptor.

The invention is described using the following definitions unless otherwise indicated.

The term “pharmaceutically acceptable derivative” means any pharmaceutically acceptable salt, ester, salt of such ester or solvate of the compounds of formula (I), or any other compound which upon administration to the recipient is capable of providing (directly or indirectly) a compound of formula (I) or an active metabolite or residue thereof.

It will be appreciated by those skilled in the art that compounds of formula (I) may be modified to provide pharmaceutically acceptable derivatives thereof at any of the functional groups in the compounds, and that the compounds of formula (I) may be derivatised at more than one position.

It will be appreciated that, for pharmaceutical use, the salts referred to above will be physiologically acceptable salts, but other salts may find use, for example in the preparation of compounds of formula (I) and the physiological acceptable salts thereof. Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropyl amine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Preferred examples of pharmaceutically acceptable salts include the ammonium, calcium, magnesium, potassium, and sodium salts, and those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The terms ‘halogen or halo’ are used to represent fluorine, chlorine, bromine or iodine.

The term ‘alkyl’ as a group or part of a group means a straight or branched chain alkyl group or combinations thereof, for example a methyl, ethyl, n-propyl, i-propyl, n-butyl, s-butyl, t-butyl, pentyl, hexyl, 1,1-dimethylethyl, or combinations thereof.

The term ‘alkoxy’ as a group or as part of a group means a straight, branched or cyclic chain alkyl group having an oxygen atom attached to the chain, for example a methoxy, ethoxy, n-propoxy, i-propoxy, n-butoxy, s-butoxy, t-butoxy group, pentoxy, hexyloxy group, cyclopentoxy or cyclohexyloxy group.

The term ‘cycloalkyl’ means a closed non-aromatic carbon ring, for example cyclobutyl, cyclopentyl, cyclohexyl or cycloheptyl, or cyclooctyl.

The term ‘alkenyl’ as a group or part of a group means a straight or branched chain carbon chain or combinations containing 1 or more double bonds for example an ethenyl, n-propenyl, i-propenyl, butenyl, pentenyl, hexenyl or combinations thereof.

The term ‘cycloalkenyl’ means a closed non-aromatic carbon ring containing 1 or more double bonds, for example cyclobutenyl, cyclopentenyl, cyclohexenyl or cycloheptenyl, or cyclooctenyl.

The term ‘alkynyl’ as a group or part of a group means a straight or branched chain carbon chain or combinations containing 1 or more triple carbon bonds for example a ethynyl, propynyl, butynyl, pentynyl, hexynyl or combinations thereof.

The term ‘cycloalkynyl’ means a closed non-aromatic carbon ring containing 1 or more triple bonds, for example cyclobutynyl, cyclopentynyl, cyclohexynyl or cycloheptynyl, or cyclooctynyl.

When R¹ and R² taken together with the N to which they are attached form an optionally substituted heterocyclyl ring, the ring may optionally contain 1, 2, 3 or 4 further hetero atoms. The ring may be saturated or unsaturated. Preferably the further hetero atoms are selected from oxygen, nitrogen or sulphur. An example of a 4-membered heterocyclyl ring is azetidinyl Examples of 5-membered heterocyclyl rings include pyrrolidinyl, Examples of 6-membered heterocyclyl rings are morpholinyl, piperazinyl or piperidinyl. An additional example is tetrahydropyridinyl. Examples of a 7-membered heterocyclyl ring are azapine or oxapine. Examples of 8-membered heterocyclyl rings are azacyclooctanyl, azaoxacyclooctanyl or azathiacyclooctanyl.

When R³ is an optionally substituted non-aromatic heterocyclyl group, the ring may contain 1, 2, 3, or 4 hetero atoms. Preferably the hetero atoms are selected from oxygen, nitrogen or sulphur. Examples of 4-membered groups are 2- or 3-azetidinyl, oxetanyl, thioxetanyl, thioxetanyl-s-oxide and thioxetanyl-s,s-dioxide. Examples of 5-membered heterocyclyl groups in this instance include dioxalanyl, pyrrolidinyl, tetrahydrofuranyl and tetrahydrothiophenyl. Additionally it can be tetrahydrothiophenyl-s,s-dioxide. Examples of 6-membered heterocyclyl groups are morpholinyl, piperidinyl, piperazinyl, tetrahydropyranyl, tetrahydrothiopyranyl, thiomorpholinyl and thiomorpholinyl-s,s-dioxide. Additional examples are tetrahydropyridinyl, dioxanyl, and tetrahydro-thiopyran 1,1 dioxide. Examples of a 7-membered heterocyclyl ring are azapine or oxapine. Examples of 8-membered groups are azacyclooctanyl, azaoxacyclooctanyl or azathiacyclooctanyl, oxacylcooctanyl, or thiacyclooctanyl.

In one particular embodiment compounds of the present invention can be selected from:
6-(3-Chloro-phenyl-amino)-4-isopropyl-N-(tetrahydro-pyran-4-ylmethyl)-nicotinamide;
6-(3-Bromo-phenyl-amino)-4-isopropyl-N-(tetrahydro-pyran-4-ylmethyl)-nicotinamide;
6-(2,4-Dichloro-phenylamino)-4-isopropyl-N-(tetrahydro-pyran-4-ylmethyl)-nicotinamide;
4-isopropyl-N-(tetrahydro-pyran-4-ylmethyl)-6-(3-trifluoromethoxy-phenylamino)-nicotinamide;
4-tert-Butyl-6-(2,4-di-chloro-phenylamino)-N-(tetrahydro-pyran-4-ylmethyl)-nicotinamide;
6-(3-Chloro-4-cyano-phenylamino)-4-isopropyl-N-(tetrahydro-pyran-4-ylmethyl)-nicotinamide;
6-(2-Fluoro-3-trifluoromethyl-phenylamino)-4-isopropyl-N-(tetrahydro-pyran-4-ylmethyl)-nicotinamide;
6-(4-Bromo-2-chloro-phenylamino)-4-isopropyl-N-(tetrahydro-pyran-4-ylmethyl)-nicotinamide;
6-(3,4-Dichloro-phenylamino)-4-isopropyl-N-(tetrahydro-pyran-4-ylmethyl)-nicotinamide;
6-(2-Bromo-4-trifluoromethoxy-phenylamino)-4-isopropyl-N-(tetrahydro-pyran-4-ylmethyl)-nicotinamide;
6-(3,5-Difluoro-phenylamino)-4-isopropyl-N-(tetrahydro-pyran-4-ylmethyl)-nicotinamide;
6-(2,4-Dichloro-phenylamino)-N-(tetrahydro-pyran-4-ylmethyl)-4-trifluoromethyl-nicotinamide;
   and pharmaceutically acceptable derivatives thereof.

Compounds of formula (I) can be prepared as set forth in scheme 1: 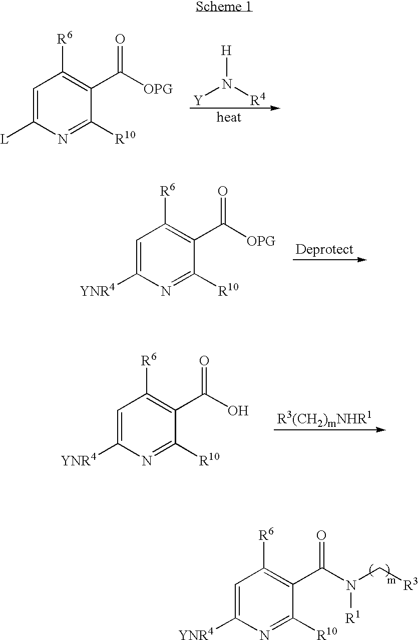
wherein L is a leaving group, for example halo, PG is a protecting group for example methyl, ethyl or benzyl, and R¹, R³, R⁴, R⁶, R¹⁰, m and Y are as defined for compounds of formula (I).

Alternatively compounds of formula (I) can be prepared as shown in scheme 2.

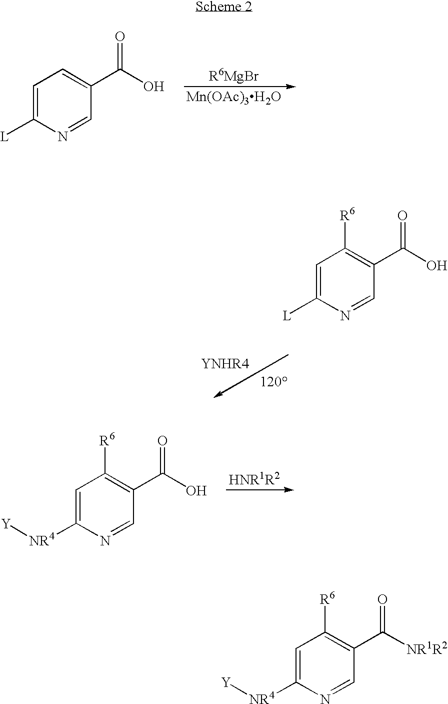

wherein L is a leaving group, for example halo e.g. chloro, and R¹, R², R⁴ and Y are as defined for compounds of formula (I).

Furthermore compounds of formula (I) when R¹⁰ is unsubstituted or substituted (C₁₋₆)alkyl or chloro and R⁶ is hydrogen can be prepared as shown in scheme 3.

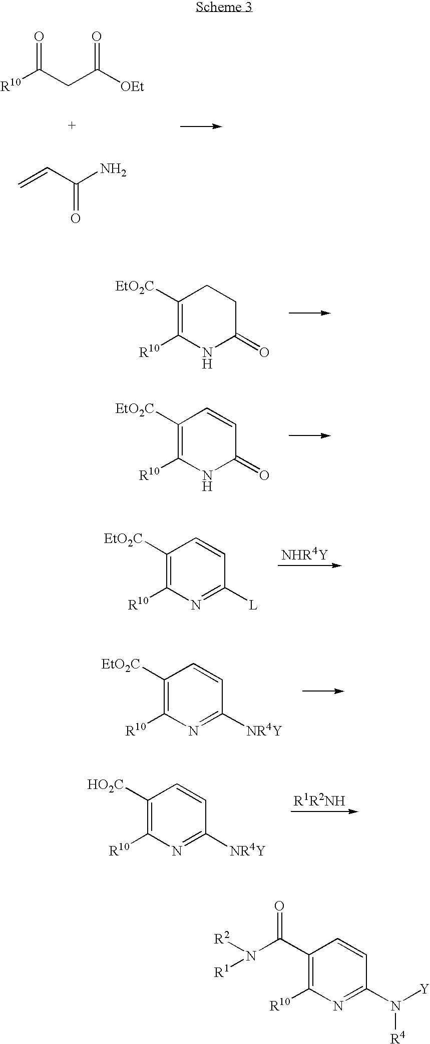

wherein L is a leaving group for example halogen, e.g. chloro, R¹, R², Y, R⁴ are as defined for compounds of formula (I).

Furthermore compounds of formula (I) when R¹⁰ is unsubstituted or substituted (C₁₋₆)alkyl or chloro and R⁶ is hydrogen can be prepared as shown in scheme 4.

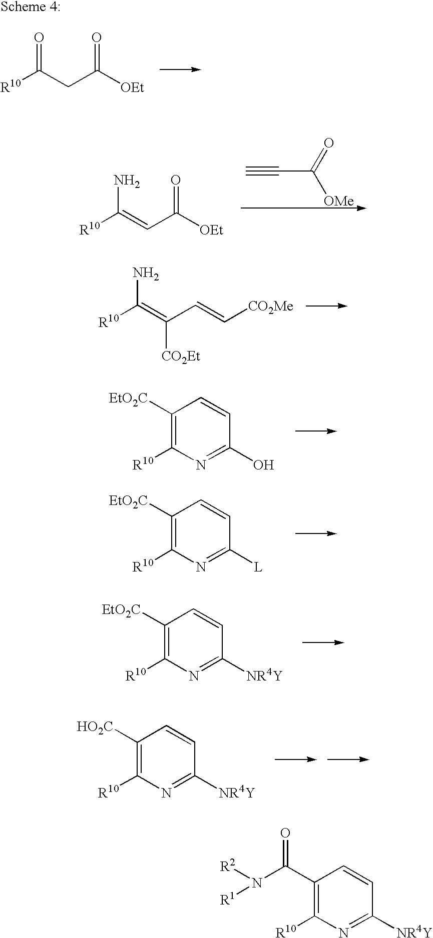

wherein L is a leaving group for example halogen, e.g. chloro, R¹, R², Y, R⁴ are as defined for compounds of formula (I).

It is to be understood that the present invention encompasses all isomers of compounds of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I and following of this invention can generally be prepared by carrying out the procedures disclosed in the Schemes and/or in the Examples below, by substituting a readily available isotopically labeled reagent for a non-isotopically labeled reagent.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

The compounds of the invention bind selectively to the CB2 receptor, and are therefore useful in treating CB2 receptor mediated diseases.

In view of their ability to bind to the CB2 receptor, the compounds of the invention may be useful in the treatment of the disorders that follow. Thus, the compounds of formula (I) may be useful as analgesics. For example they may be useful in the treatment of chronic inflammatory pain (e.g. pain associated with rheumatoid arthritis, osteo-arthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; dysmenorrhea, chronic pain, dental pain algesia, pelvic pain, poststroke pain and menstrual pain.

The compounds of the invention may also be useful disease modification or joint structure preservation in multiple sclerosis, rheumatoid arthritis, osteo-arthritis, rheumatoid spondylitis, gouty arthritis and juvenile arthritis.

The compounds of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of formula (I) may also be useful in the treatment of fever.

The compounds of formula (I) may also be useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); cough, gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastroesophageal reflux disease emesis, oesophagitis, organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, polymyositis, gingivitis, myocardial ischemia, pyrexia, systemic lupus erythematosus, tendinitis, bursitis, and Sjogren's syndrome.

The compounds of formula (I) may also be useful in the treatment of bladder hyperrelexia following bladder inflammation.

The compounds of formula (I) are also useful in the treatment of immunological diseases such as autoimmune diseases, immunological deficiency diseases or organ transplantation. The compounds of formula (I) are also effective in increasing the latency of HIV infection.

The compounds of formula (I) are also useful in the treatment of diseases of abnormal platelet function (e.g. occlusive vascular diseases).

The compounds of formula (I) are also useful in the treatment of neuritis, heart burn, dysphagia, pelvic hypersensitivity, urinary incontinence, cystitis or pruritis.

The compounds of formula (I) are also useful for the preparation of a drug with diuretic action.

The compounds of formula (I) are also useful in the treatment of impotence or erectile dysfunction.

The compounds of formula (I) are also useful for attenuating the hemodynamic side effects of non-steroidal anti-inflammatory drugs (NSAID's) and cyclooxygenase-2 (COX-2) inhibitors.

The compounds of formula (I) are also useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with ageing, particularly Age Associated Memory Impairment. The compounds may also be useful for the treatment of amyotrophic lateral sclerosis (ALS) and neuroinflamation.

The compounds of formula (I) are also useful in neuroprotection and in the treatment of neurodegeneration following stroke, cardiac arrest, pulmonary bypass, traumatic brain injury, spinal cord injury or the like.

The compounds of formula (I) are also useful in the treatment of tinnitus.

The compounds of formula (I) are also useful in the treatment of psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), panic disorder, agoraphobia, social phobia, obsessive compulsive disorder and post-traumatic stress disorder, memory disorders, including dementia, amnesic disorders and age-associated memory impairment, disorders of eating behaviours, including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof.

The compounds of formula (I) are also useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

The compounds of formula (I) are also useful in the treatment of kidney dysfunction (nephritis, particularly mesangial proliferative glomerulonephrltis, nephritic syndrome), liver dysfunction (hepatitis, cirrhosis), gastrointestinal dysfunction (diarrhoea) and colon cancer.

The compounds of formula (I) may useful for the treatment of bladder hyper-reflexia following bladder inflammation.

It is to be understood that references to treatment includes both treatment of established symptoms and prophylactic treatment unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the activity of cannabinoid 2 receptors.

According to a further aspect of the invention, we provide a method of treating a human or animal subject suffering from a condition which is mediated by the activity of cannabinoid 2 receptors which comprises administering to said subject a therapeutically effective amount of a compound of formula (I) or a pharmaceutically acceptable derivative thereof.

According to a further aspect of the invention we provide a method of treating a human or animal subject suffering from an immune disorder, an inflammatory disorder, pain, rheumatoid arthritis, multiple sclerosis, osteoarthritis or osteoporosis which method comprises administering to said subject an effective amount of a compound of formula (I) or a pharmaceutically acceptable derivative thereof. Preferably the pain is selected from inflammatory pain, viseral pain, cancer pain, neuropathic pain, lower back pain, muscular sceletal, post operative pain, acute pain and migraine. More preferably the inflammatory pain is pain associated with rheumatoid arthritis or osteoarthritis.

According to another aspect of the invention is provided the use of a compound of formula (I) or a pharmaceutically acceptable derivative thereof for the manufacture of a therapeutic agent for the treatment or prevention of a condition such as an immune disorder, an inflammatory disorder, pain, rheumatoid arthritis, multiple sclerosis, osteoarthritis or osteoporosis

Preferably the pain is selected from inflammatory pain, viseral pain, cancer pain, neuropathic pain, lower back pain, muscular sceletal, post operative pain, acute pain and migraine. More preferably the inflammatory pain is pain associated with rheumatoid arthritis or osteoarthritis.

In order to use a compound of formula (I) or a pharmaceutically acceptable derivative thereof for the treatment of humans and other mammals it is normally formulated in accordance with standard pharmaceutical practice as a pharmaceutical composition. Therefore in another aspect of the invention is provided a pharmaceutical composition comprising a compound of formula (I) or a pharmaceutically acceptable derivative thereof adapted for use in human or veterinary medicine.

As used herein, “modulator” means both antagonist, full or partial agonist and inverse agonist. In one embodiment of the present modulators are agonists.

The term “treatment” or “treating” as used herein includes the treatment of established disorders and also includes the prophylaxis thereof. The term “prophylaxis” is used herein to mean preventing symptoms in an already afflicted subject or preventing recurrence of symptoms in an afflicted subject and is not limited to complete prevention of an affliction.

Compounds of formula (I) and their pharmaceutically acceptable derivatives may be administered in a standard manner for the treatment of the indicated diseases, for example orally, parentarally, sub-lingually, dermally, intranasally, transdermally, rectally, via inhalation or via buccal administration.

Compositions of formula (I) and their pharmaceutically acceptable derivatives which are active when given orally can be formulated as syrups, tablets, capsules and lozenges. A syrup formulation will generally consist of a suspension or solution of the compound or salt in a liquid carrier for example, ethanol, peanut oil, olive oil, glycerine or water with a flavouring or colouring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or derivative in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of formula (I) or a pharmaceutically acceptable derivative thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

Each dosage unit for oral administration contains suitably from 0.01 mg/Kg to 500 mg/Kg for example 0.1 mg to 500 mg/Kg, and preferably from 0.01 mg to 100 mg/Kg for example 1 mg/Kg to 100 mg/Kg, and each dosage unit for parenteral administration contains suitably from 0.1 mg to 100 mg/Kg, of a compound of formula (I) or a pharmaceutically acceptable derivative thereof calculated as the free acid. Each dosage unit for intranasal administration contains suitably 1-400 mg and preferably 10 to 200 mg per person. A topical formulation contains suitably 0.01 to 5.0% of a compound of formula (I).

The daily dosage regimen for oral administration is suitably about 0.01 mg/Kg to 40 mg/Kg, of a compound of formula (I) or a pharmaceutically acceptable derivative thereof calculated as the free acid. The daily dosage regimen for parenteral administration is suitably about 0.001 mg/Kg to 40 mg/Kg, of a compound of formula (I) or a pharmaceutically acceptable derivative thereof calculated as the free acid. The daily dosage regimen for intranasal administration and oral inhalation is suitably about 10 to about 500 mg/person. The active ingredient may be administered from 1 to 6 times a day, sufficient to exhibit the desired activity.

It may be advantageous to prepare the compounds of the present invention as nanoparticles. This may improve the oral bioavailability of the compounds. For the purposes of the present invention “nanoparticulate” is defined as solid particles with 50% of the particles having a particle size of less than 1 μm, more preferably less than 0.75 μm

The particle size of the solid particles of compound (I) may be determined by laser diffraction. A suitable machine for determining particle size by laser diffraction is a Lecotrac laser particle size analyser, using an HELOS optical bench fitted with a QUIXEL dispersion unit.

Numerous processes for the synthesis of solid particles in nanoparticulate form are known. Typically these processes involve a milling process, preferably a wet milling process in the presence of a surface modifying agent that inhibits aggregation and/or crystal growth of the nanoparticles once created. Alternatively these processes may involve a precipitation process, preferably a process of precipitation in an aqueous medium from a solution of the drug in a non-aqueous solvent.

Accordingly, in a further aspect, the present invention provides a process for preparing compound (I) in nanoparticulate form as hereinbefore defined, which process comprises milling or precipitation.

Representative processes for the preparation of solid particles in nanoparticulate form are described in the patents and publications listed below.

U.S. Pat. No. 4,826,689 to Violanto & Fischer, U.S. Pat. No. 5,145,684 to Liversidge et al U.S. Pat. No. 5,298,262 to Na & Rajagopalan, U.S. Pat. No. 5,302,401 Liversidge et al U.S. Pat. No. 5,336,507 to Na & Rajagopalan, U.S. Pat. No. 5,340,564 to Illig & Sarpotdar U.S. Pat. No. 5,346,702 to Na Rajagopalan, U.S. Pat. No. 5,352,459 to Hollister et al U.S. Pat. No. 5,354,560 to Lovrecich, U.S. Pat. No. 5,384,124 to Courteille et al, U.S. Pat. No. 5,429,824 to June, U.S. Pat. No. 5,503,723 to Ruddy et al, U.S. Pat. No. 5,510,118 to Bosch et al, U.S. Pat. No. 5,518 to Bruno et al, U.S. Pat. No. 5,518,738 to Eickhoff et al, U.S. Pat. No. 5,534,270 to De Castro, U.S. Pat. No. 5,536,508 to Canal et al, U.S. Pat. No. 5,552,160 to Liversidge et al, U.S. Pat. No. 5,560,931 to Eickhoff et al, U.S. Pat. No. 5,560,932 to Bagchi et al, U.S. Pat. No. 5,565,188 to Wong et al, U.S. Pat. No. 5,571,536 to Eickhoff et al, U.S. Pat. No. 5,573,783 to Desieno & Stetsko, U.S. Pat. No. 5,580,579 to Ruddy et al, U.S. Pat. No. 5,585,108 to Ruddy et al, U.S. Pat. No. 5,587,143 to Wong, U.S. Pat. No. 5,591,456 to Franson et al, U.S. Pat. No. 5,622,938 to Wong, U.S. Pat. No. 5,662,883 to Bagchi et al, U.S. Pat. No. 5,665,331 to Bagchi et al, U.S. Pat. No. 5,718,919 to Ruddy et al, U.S. Pat. No. 5,747,001 to Wiedmann et al, WO93/25190, WO96/24336, WO 97/14407, WO 98/35666, WO 99/65469, WO 00/18374, WO 00/27369, WO 00/30615 and WO 01/41760.

Such processes may be readily adapted for the preparation of compound (I) in nanoparticulate form. Such processes form a further aspect of the invention.

The process of the present invention preferably uses a wet milling step carried out in a mill such as a dispersion mill in order to produce a nanoparticulate form of the compound. The present invention may be put into practice using a conventional wet milling technique, such as that described in Lachman et al., The Theory and Practice of Industrial Pharmacy, Chapter 2, “Milling” p. 45 (1986).

In a further refinement, WO02/00196 (SmithKline Beecham pic) describes a wet milling procedure using a mill in which at least some of the surfaces are made of nylon (polyamide) comprising one or more internal lubricants, for use in the preparation of solid particles of a drug substance in nanoparticulate form.

In another aspect the present invention provides a process for preparing compounds of the invention in nanoparticulate form comprising wet milling a suspension of compound in a mill having at least one chamber and agitation means, said chamber(s) and/or said agitation means comprising a lubricated nylon, as described in WO02/00196.

The suspension of a compound of the invention for use in the wet milling is typically a liquid suspension of the coarse compound in a liquid medium. By “suspension” is meant that the compound is essentially insoluble in the liquid medium. Representative liquid media include an aqueous medium. Using the process of the present invention the average particle size of coarse compound of the invention may be up to 1 mm in diameter. This advantageously avoids the need to pre-process the compound.

In a further aspect of the invention the aqueous medium to be subjected to the milling comprises compound (I) present in from about 1% to about 40% w/w, preferably from about 10% to about 30% w/w, more preferably about 20% w/w.

The aqueous medium may further comprise one or more pharmaceutically acceptable water-soluble carriers which are suitable for steric stabilisation and the subsequent processing of compound (I) after milling to a pharmaceutical composition, e.g. by spray drying. Pharmaceutically acceptable excipients most suitable for steric stabilisation and spray-drying are surfactants such as poloxamers, sodium lauryl sulphate and polysorbates etc; stabilisers such as celluloses e.g. hydroxypropylmethyl cellulose; and carriers such as carbohydrates e.g. mannitol.

In a further aspect of the invention the aqueous medium to be subjected to the milling may further comprise hydroxypropylmethyl cellulose (HPMC) present from about 0.1 to about 10% w/w.

The process of the present invention may comprise the subsequent step of drying compound of the invention to yield a powder.

Accordingly, in a further aspect, the present invention provides a process for preparing a pharmaceutical composition contain a compound of the present invention which process comprises producing compound of formula (I) in nanoparticulate form optionally followed by drying to yield a powder.

A further aspect of the invention is a pharmaceutical composition comprising a compound of formula (I) or a pharmaceutically acceptable derivative thereof in which the compound of formula (I) or a pharmaceutically acceptable derivative thereof is present in solid particles in nanoparticulate form, in admixture with one or more pharmaceutically acceptable carriers or excipients.

By “drying” is meant the removal of any water or other liquid vehicle used during the process to keep compound of formula (I) in liquid suspension or solution. This drying step may be any process for drying known in the art, including freeze drying, spray granulation or spray drying. Of these methods spray drying is particularly preferred. All of these techniques are well known in the art. Spray drying/fluid bed granulation of milled compositions is carried out most suitably using a spray dryer such as a Mobile Minor Spray Dryer [Niro, Denmark], or a fluid bed drier, such as those manufactured by Glatt, Germany.

In a further aspect the invention provides a pharmaceutical composition as hereinbefore defined, in the form of a dried powder, obtainable by wet milling solid particles of compound of formula (I) followed by spray-drying the resultant suspension.

Preferably, the pharmaceutical composition as hereinbefore defined, further comprises HPMC present in less than 15% w/w, preferably in the range 0.1 to 10% w/w.

The CB₂ receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as aspirin, diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; EP₁ receptor ligands, EP₄ receptor ligands; EP₂ receptor ligands; EP₃ receptor ligands; EP₄ antagonists; EP₂ antagonists and EP₃ antagonists; bradykinin receptor ligands and vanilloid receptor ligand, antirheumatoid arthritis drugs, for example anti TNF drugs e.g. enbrel, remicade, anti-IL-1 drugs, or DMARDS e.g. leflunamide. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The compound of the present invention may be administered in combination with other active substances such as 5HT3 antagonists, NK-1 antagonists, serotonin agonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compound of the inventions include for example ondansetron, granisetron, metoclopramide.

Suitable serotonin agonists which may be used in combination with the compound of the invention include sumatriptan, rauwolscine, yohimbine, metoclopramide.

Suitable SSRIs which may be used in combination with the compound of the invention include fluoxetine, citalopram, femoxetine, fluvoxamine, paroxetine, indalpine, sertraline, zimeldine.

Suitable SNRIs which may be used in combination with the compound of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

It will be appreciated that the compounds of any of the above combinations or compositions may be administered simultaneously (either in the same or different pharmaceutical formulations), separately or sequentially.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

Determination of Cannabinoid CB1 Receptor Agonist Activity

The cannabinoid CB1 receptor agonist activity of the compounds of formula (I) was determined in accordance with the following experimental method.

Experimental Method

Yeast (Saccharomyces cerevisiae) cells expressing the human cannabinoid CB1 receptor were generated by integration of an expression cassette into the ura3 chromosomal locus of yeast strain MMY23. This cassette consisted of DNA sequence encoding the human CB1 receptor flanked by the yeast GPD promoter to the 5′ end of CB1 and a yeast transcriptional terminator sequence to the 3′ end of CB1. MMY23 expresses a yeast/mammalian chimeric G-protein alpha subunit in which the C-terminal 5 amino acids of Gpa1 are replaced with the C-terminal 5 amino acids of human Gαi3 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM stocks in DMSO. EC₅₀ values (the concentration required to produce 50% maximal response) were estimated using dilutions of between 3- and 5-fold (BiomekFX, Beckman) into DMSO. Agonist solutions in DMSO (1% final assay volume) were transferred into black, clear bottom, microtitre plates from NUNC (96- or 384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 20 μM fluorescein di-β-D-glucopyranoside (FDGlu). This mixture (50 ul per well for 384-well plates, 200 ul per well for 96-well plates) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a Spectrofluor microtitre plate reader (Tecan; excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation
Eₘₐₓ=Max_{[compound X]}−Min_{[compound X]}/Max_{[HU210]}−Min_{[HU210]}×100%

where Max_{[compound X]} and Min_{[compound X] are the fitted maximum and minimum respectively from the concentration effect curve for compound X, and Max}_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation
EMR=EC_{50 [compound X]}/EC_{50 [HU210]}

Where EC_{50 [compound X]} is the EC₅₀ of compound X and EC_{50 [HU210]} is the EC50 of HU210.

Compounds of the Examples tested according to this method had EC₅₀ values >30,000 nM at the cloned human cannabinoid CB1 receptor.

Determination of Cannabinoid CB2 Receptor Agonist Activity

The cannabinoid CB2 receptor agonist activity of the compounds of formula (I) was determined in accordance with the following experimental method.

Experimental Method

Yeast (Saccharomyces cerevisiae) cells expressing the human cannabinoid CB2 receptor were generated by integration of an expression cassette into the ura3 chromosomal locus of yeast strain MMY23. This cassette consisted of DNA sequence encoding the human CB2 receptor flanked by the yeast GPD promoter to the 5′ end of CB2 and a yeast transcriptional terminator sequence to the 3′ end of CB2. MMY23 expresses a yeast/mammalian chimeric G-protein alpha subunit in which the C-terminal 5 amino acids of Gpa1 are replaced with the C-terminal 5 amino acids of human Gαi3 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM stocks in DMSO. EC₅₀ values (the concentration required to produce 50% maximal response) were estimated using dilutions of between 3- and 5-fold (BiomekFX, Beckman) into DMSO. Agonist solutions in DMSO (1% final assay volume) were transferred into black, clear bottom, microtitre plates from NUNC (96- or 384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 20M fluorescein di-β-D-glucopyranoside (FDGlu). This mixture (50 ul per well for 384-well plates, 200 ul per well for 96-well plates) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a Spectrofluor microtitre plate reader (Tecan; excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation
Eₘₐₓ=Max_{[compound X]}−Min_{[compound X]}/Max_{[HU210]}−Min_{[HU210]}×100%

where Max_{[compound X]} and Min_{[compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation
EMR=EC_{50 [compound X]}/EC_{50 [HU210]}

Where EC_{50 [compound X]} is the EC₅₀ of compound X and EC_{50 [HU210]} is the EC₅₀ of HU210.

The compounds of Examples 1 to 38, 50 to 55, 69 to 93, 104 to 172, 204, 208 to 220, 223, 224, 234 to 279, 293, 295 to 297 tested according to this method had an EC₅₀ values of <300 nM and efficacy value of >50% at the cloned human cannabinoid CB2 receptor.

The compounds of Examples 39 to 45, 56 to 62, 94 to 102, 173 to 177, 280 to 292, 294 and 298 to 304 tested according to this method had an EC₅₀ values of <1000 nM and efficacy value of >50% at the cloned human cannabinoid CB2 receptor.

The compounds of Examples 46 to 49, 63 to 68, 103, 178 to 203, 205 to 207, 222, 225 to 233 and 305 tested according to this method had an EC₅₀ values of >1000 nM and/or efficacy value of <50% at the cloned human cannabinoid CB2 receptor.

The compound of Examples 221 tested according to this method had an EC₅₀ value of between 300 and 1000 nM and an efficacy value of <30% at the cloned human cannabinoid CB2 receptor.

The following examples are illustrative, but not limiting of the embodiments of the present invention.

The following abbreviations are used herein

MDAP represents mass-directed auto-purification;

THF represents tetrahydrofuran;

DCM represents dichloromethane;

DMSO represents dimethyl sulfoxide;

TFA represents trifluoroacetic acid.

All NMR experimental data was recorded at 400 MHz unless indicated.

Conditions, Hardware, and Software Used for Mass-Directed Autopurification

Hardware

Waters 600 gradient pump, Waters 2700 sample manager, Waters Reagent Manager, Micromass ZMD mass spectrometer, Gilson 202—fraction collector, Gilson Aspec—waste collector.

Software

Micromass Masslynx version 3.5

Column

The column used is typically a Supelco ABZ+ column whose dimensions are 10 mm internal diameter by 100 mm in length. The stationary phase particle size is 5 μm.

Solvents

A. Aqueous solvent=Water+0.1% Formic Acid

B. Organic solvent=MeCN: Water 95:5+0.05% Formic Acid

Make up solvent=MeOH: Water 80:20+50 mMol Ammonium Acetate

Needle rinse solvent=MeOH: Water: DMSO 80:10:10

Methods

Five methods are used depending on the analytical retention time of the compound of interest.

They all have a flow rate of 20 ml/min and a 15-minute runtime, which comprises of a 10-minute gradient followed by a 5-minute column flush and re-equilibration step.

Method 1 MDAP 1.5-2.2=0-30% B

Method 2 MDAP 2.0-2.8=5-30% B

Method 3 MDAP 2.5-3.0=15-55% B

Method 4 MDAP 2.8-4.0=30-80% B

Method 5 MDAP 3.8-5.5=50-90% B

Method Used for Purification Using the Biotage Horizon System.

Column: Biotage C18HS 25 + S
Fraction volume: 9 ml; UV Threshold: 0.03 AU
Solvent A = Water, B = Acetonitrile, Gradient:

| Volume(ml) | A | B |
|---|---|---|
| 0 | 70% | 30% |
| 240 | 0% | 100% |

### Description 1: Methyl 6-(3-chlorophenylamino)-4-(trifluoromethyl)-nicotinate

A mixture of methyl 6-chloro-4-(trifluoromethyl)-nicotinate (0.7 g, ex Fluorochem) and 3-chloroaniline (0.62 mL) was heated at 120° C. for 6 h. The reaction mixture solidified and the crude crystals were used for the next step without further purification.

LC-MS (ESI+): t=10.20 min, (MH+) 331 and 333.

### Description 2: 6-(3-Chlorophenylamino)-4-(trifluoromethyl)-nicotinic acid hydrochloride

To a suspension of methyl 6-(3-chlorophenylamino)-4-(trifluoromethyl)-nicotinate (Description 1) (1.0 g) in ethanol (5 mL) was added a solution of potassium hydroxide (510 mg) in water (5 mL) and the solution was stirred at reflux for 30 min. After removal of the ethanol under reduced pressure, the mixture was diluted with water (10 mL) and washed twice with dichloromethane. Concentrated hydrochloric acid was added to adjust pH to 1 and the precipitated solid was filtered and dried in vacuo at 60° C. to afford 6-(3-chlorophenylamino)-4-(trifluoromethyl)-nicotinic acid as its hydrochloride salt (0.62 g).

LC-MS (ESI+): t=8.51 min, (MH+) 317 and 319.

### Description 3: 6-Chloro-N-cyclohexylmethyl-nicotinamide

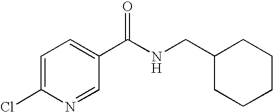

To a solution of 6-chloronicotinoyl chloride (1.5 g, ex Lancaster) in dry dichloromethane (15 ml) was added dropwise at 0° under nitrogen a solution of cyclohexanemethanamine (1.11 ml, ex Lancaster) and triethylamine (1.5 ml) in dry dichloromethane (15 ml) over 1 hour. The solution was stirred at 0° for 1 hour. Dichloromethane was removed under reduced pressure and ethyl acetate (30 ml) added. The solution was washed with water (3×20 ml), dried (MgSO₄) and evaporated to afford 6-chloro-N-cyclohexylmethyl-nicotinamide (1.96 g).

NMR (DMSO-d6) δ 0.85-1.0 (2H, m), 1.1-1.25 (3H, m), 1.54 (1H, m), 1.55-1.75 (5H, m), 3.11 (2H, t), 7.64 (1H, d), 8.23 (1H, d of d), 8.69 (1H, t), 8.82 (1H, s).

LC/MS t=2.9 min, Molecular ion observed [MH⁺] 253 consistent with molecular formula C₁₃H₁₇ ³⁵ClN₂O

### Description 4: 6-Chloro-N-cyclohexylmethyl-4-isopropyl-nicotinamide

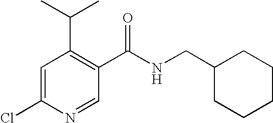

To a solution of 6-chloro-N-cyclohexylmethyl-nicotinamide (Description 3) (0.89 g) in dry tetrahydrofuran (5 ml) was added dropwise at 0° under nitrogen a 2.0M solution of isopropylmagnesium chloride (5.3 ml, ex Aldrich) and the solution stirred at room temperature for 15 hours. It was cooled to 0° and dry methanol (0.86 ml) added dropwise and the solution stirred for 15 minutes. 2,3-Dichloro-5,6-dicyano-1,4-benzoquinone (0.88 g) was added and the mixture stirred at room temperature for 30 minutes then evaporated under reduced pressure to ca. 6 ml. The residual liquid was warmed to 50° and t-butyl methyl ether (20 ml) added. The mixture was stirred under reflux for 1 hour then at room temperature for 1 hour and filtered. The filtrate was evaporated and the residue purified using Biotage chromatography (Merck 9385 silica gel) with 1:4 ethyl acetate:isohexane to afford 6-chloro-N-cyclohexylmethyl-4-isopropyl-nicotinamide (886 mg).

NMR (DMSO-d6) δ 0.85-1.0 (2H, m), 1.1-1.25 (3H, m), 1.19 (6H, d), 1.50 (1H, m), 1.55-1.75 (5H, m), 3.08 (2H, t), 3.22 (1H, m), 7.53 (1H, s), 8.24 (1H, s), 8.57 (1H, t).

LC/MS, t=3.2 min, Molecular ion observed [MH⁺]=295 consistent with the molecular formula C₁₆H₂₃ ³⁵ClN₂O.

### Description 5: 6-Chloro-N-cyclobutylmethyl-nicotinamide

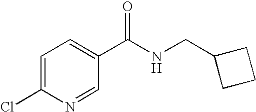

Prepared in a manner similar to Description 3 from 6-chloronicotinoyl chloride (1.9 g, ex-Lancaster), C-cyclobutyl-methylamine hydrochloride (1.52 g), and triethylamine (3.4 ml), to give the title compound (2.02 g).

NMR (DMSO-d6) δ 1.71 (2H, m), 1.82 (2H, m), 1.99 (2H, m), 2.52 (1H, m excess), 3.31 (2H, t), 7.64 (1H, d), 8.22 (1H, d of d), 8.71 (1H, t), 8.81 (1H, d).

LC/MS t=2.51 min, Molecular ion observed [MH⁺]=225 consistent with the molecular formula C₁₁H₁₃³⁵ClN₂O

### Description 6: 6-Chloro-N-cyclobutylmethyl-4-isopropyl-nicotinamide

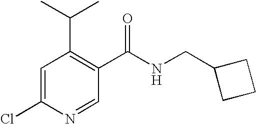

Prepared in a manner similar to Description 4 from 6-chloro-N-cyclobutylmethyl-nicotinamide (Description 3) (2.00 g), and 2.0M isopropylmagnesium chloride in THF (13.5 ml), to give the title compound (1.31 g).

NMR (DMSO-d6) δ 1.19 (6H, d), 1.72 (2H, m), 1.82 (2H, m), 1.98 (2H, m), 2.50 (1H, m excess), 3.20 (1H, m), 3.27 (2H, t), 7.53 (1H, s), 8.23 (1H, s), 8.58 (1H, t).

LC/MS t=3.07 min, [MH+]=267 consistent with the molecular formula C₁₄H₁₉³⁵ClN₂O

### Description 7: 6-Chloro-N-(tetrahydro-pyran-4-ylmethyl)-nicotinamide

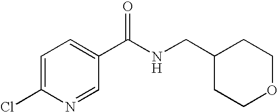

In a manner similar to Description 5,6-chloronicotinoyl chloride (1.90 g) and C-(tetrahydro-pyran-4-yl)-methylamine (1.65 g) afforded the title compound (1.46 g).

NMR (DMSO-d6) δ 1.1-1.25 (2H, m), 1.60 (2H, d), 1.79 (1H, m), 3.17 (2H, t), 3.26 (2H, t), 3.83 (2H, d of d), 7.64 (1H, d), 8.23 (1H, d of d), 8.75 (1H, t), 8.82 (1H, s).

LC/MS t=2.1 min, [MH⁺] 255 consistent with the molecular formula C₁₂H₁₅³⁵ClN₂O₂

### Description 8: 6-Chloro-4-isopropyl-N-(tetrahydro-pyran-4-ylmethyl)-nicotinamide

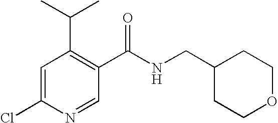

In a manner similar to Description 4,6-chloro-N-(tetrahydro-pyran-4-ylmethyl)-nicotinamide (Description 7) (1.46 g) and 2.0M isopropylmagnesium chloride in tetrahydrofuran (8.5 ml) afforded the title compound (624 mg).

NMR (DMSO-d6) δ 1.1-1.25 (2H, m), 1.19 (6H, d), 1.60 (2H, d), 1.75 (1H, m), 3.14 (2H, t), 3.21 (1H, m), 3.27 (2H, t), 3.85 (2H, d of d), 7.54 (1H, d), 8.26 (1H, s), 8.63 (1H, t).

LC/MS t=2.4 min, [MH⁺] 297 consistent with the molecular formula C₁₅H₂₁³⁵ClN₂O₂

### Description 9: 6-Chloro-N-cycloperitylmethyl-nicotinamide

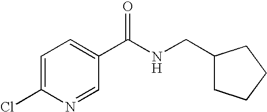

In a manner similar to Description 3,6-chloronicotinoyl chloride (0.50 g) and cyclopentanemethylamine hydrochloride (385 mg) afforded the title compound (534 mg).

NMR (DMSO-d6) δ 1.2-1.3 (2H, m), 1.45-1.65 (4H, m), 1.65-1.75 (2H, m), 2.13 (1H, m), 3.20 (2H, t), 7.64 (1H, d), 8.23 (1H, d of d), 8.74 (1H, t), 8.82 (1H, s).

LC/MS t=2.7 min, [MH⁺] 239, consistent with the molecular formula C₁₂H₁₅³⁵ClN₂O

### Description 10: 6-Chloro-N-cyclopentylmethyl-4-isopropyl-nicotinamide

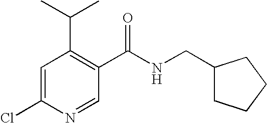

In a manner similar to Description 4,6-chloro-N-cyclopentyl-nicotinamide (Description 9)(532 mg) and 2.0M isopropylmagnesium chloride in tetrahydrofuran (3.4 ml) afforded the title compound (166 mg).

NMR (DMSO-d6)δ 1.19 (6H, d), 1.2-1.3 (2H, m), 1.45-1.65 (4H, m), 1.65-1.75 (2H, m), 2.10 (1H, m), 3.17 (2H, t), 3.21 (1H, m), 7.53 (1H, s), 8.23 (1H, s), 8.61 (1H, t).

LC/MS t=3.1 min, [MH⁺] 281, consistent with the molecular formula C₁₅H₂₁³⁵ClN₂O.

### Description 11: 1-(6-Chloro-4-isopropyl-pyridin-3-yl)-1-morpholin-4-yl-methanone

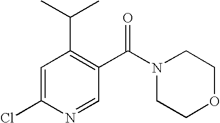

In a manner similar to Description 4,1-(6-chloro-pyridin-3-yl)-1-morpholin-4-yl-methanone (534 mg, Ref: US Patent Application 2002183309 (2002), and 2.0M isopropyl-magnesium chloride in tetrahydrofuran (3.6 ml) afforded the title compound (169 mg).

NMR (DMSO-d6) δ 1.19 (6H, t), 2.89 (1H, m), 3.1-3.25 (2H, m), 3.45 (1H, m), 3.55-3.75 (5H, m), 7.60 (1H, s), 8.26 (1H, s).

LC/MS t=2.3 min, [MH⁺] 269, consistent with the molecular formula C₁₃H₁₇³⁵ClN₂O₂

### Description 12: 6-Chloro-4-isopropyl-nicotinic acid

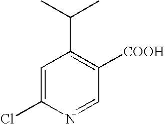

2M Isopropylmagnesium bromide in tetrahydrofuran (48 ml) was added dropwise over 1 hour to a solution of 6-chloronicotinic acid (Aldrich) (6.0 g) in dry tetrahydrofuran (100 ml) at 0° under nitrogen and the solution stirred at 0° for 3 hours then at room temperature for 15 hours. It was cooled to −60° and acetic acid (48 ml), tetrahydrofuran (40 ml) and manganese (III) acetate dihydrate (20.4 g) added successively. The mixture was stirred at −70° for 30 minutes then at room temperature for 1 hour. The suspension was filtered through Celite and the filtrate evaporated under reduced pressure. The residue was partitioned between dichloromethane (150 ml) and water (120 ml) and the aqueous layer separated and washed with dichloromethane (2×50 ml). The combined organic layers were dried (MgSO₄) and evaporated under reduced pressure to afford, after silica gel chromatography using 3:1 isohexane:ethyl acetate, 6-chloro-4-isopropyl-nicotinic acid (2.31 g).

NMR (DMSO-d6)δ 1.21 (6H, d), 3.76 (1H, m), 7.60 (1H, s), 8.67 (1H, s), 13.55 (1H, br s).

LC/MS t=2.6 min, [MH⁺] 200 consistent with molecular formula C₉H₁₀³⁵ClN0₂

### Description 13: 6-(3-Chloro-phenylamino)-4-isopropyl-nicotinic acid

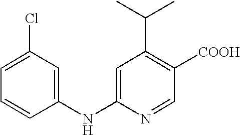

A mixture of 6-chloro-4-isopropyl-nicotinic acid (Description 12) (0.50 g) and 3-chloroaniline (265 mg) was stirred at 120° for 1½ hours. Isopropanol was added and the mixture chilled. Insoluble solid was filtered off, washed successively with isopropanol and ether and dried in vacuo at 50° to afford 6-(3-chloro-phenylamino)-4-isopropyl-nicotinic acid (0.51 g).

NMR (DMSO-d6) δ 1.19 (6H, d), 3.93 (1H, m), 6.85 (1H, s), 6.99 (1H, d), 7.31 (1H, t), 7.53 (1H, d), 8.00 (1H, s), 8.64 (1H, s), 9.73 (1H, s), 12.6 (1H, br s).

LC/MS t=3.63 min, [MH⁺] 291, consistent with molecular formula C₁₅H₁₅³⁵ClN0₂

### Description 14: 4-tert-Butyl-6-chloro-N-cyclohexylmethyl-nicotinamide

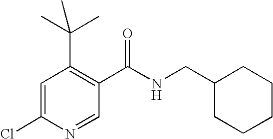

1.6 M n-Butyllithium in hexane (2.7 ml) was added dropwise to a stirred solution of 6-chloro-N-cyclohexylmethyl-4-isopropyl-nicotinamide (Description 4) (0.50 g) in dry tetrahydrofuran (3 ml) at −70° under nitrogen. The solution was stirred for 15 minutes then warmed to 0° and a solution of methyl iodide (0.11 ml) in dry tetrahydrofuran (2 ml) added, followed by stirring for a further 30 minutes. Solvent was removed under reduced pressure and ethyl acetate (10 ml) added. The solution was washed with water (10 ml), dried (MgSO₄) and evaporated under reduced pressure. The residue was purified using silica gel chromatography with 17:3 isohexane:ethyl acetate and further purified by MDAP to afford the title compound (83 mg).

NMR (CDCl₃) δ 0.95-1.05 (2H, m), 1.15-1.3 (4H, m), 1.42 (9H, s), 1.65-1.8 (5H, m), 3.28 (2H, t), 5.81 (1H, br s), 7.36 (1H, s), 8.21 (1H, s).

LC/MS t=3.6 min, [MH⁺] 309, consistent with C₁₇H₂₅³⁵ClN₂O

### Description 15: 4-tert-Butyl-6-chloro-N-(tetrahydro-pyran-4-ylmethyl)-nicotinamide

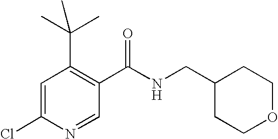

In a manner similar to Description 14, 6-chloro-4-isopropyl-N-(tetrahydro-pyran-4-ylmethyl)-nicotinamide (Description 8) (1.0 g), 1.6 M n-butyllithium in hexane (2.7 ml) and methyl iodide (0.22 ml) afforded, after silica gel chromatography, eluting with 1:1 isohexane:ethyl acetate and MDAP purification, the title compound (116 mg).

NMR (CDCl₃) δ 1.3-1.45 (2H, m), 1.42 (9H, s), 1.68 (2H, d), 1.91 (1H, m), 3.34 (2H, t), 3.40 (2H, t), 4.00 (2H, d of d), 6.04 (1H, br s), 7.36 (1H, s), 8.18 (1H, s).

LC/MS t=2.4 min, [MH⁺] 311 consistent with molecular formula C₁₆H₂₃³⁵ClN₂0₂

### Description 16: 4-Aminomethyltetrahydropyran-4-ol hydrochloride

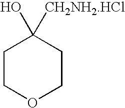

To a solution of 1.0M lithium aluminium hydride in tetrahydrofuran (20 ml) was added under a nitrogen atmosphere a solution of 4-hydroxytetra-hydropyran-4-carbonitrile (0.50 g, prepared as described in Eiden et al., Arch. Pharm., 320, 348, (1987)) in tetrahydrofuran (2 ml) and the solution stirred at reflux for 6 hours. Water (1 ml) and 2N sodium hydroxide solution (1 ml) were added cautiously and the resultant solid filtered and washed with ether. The filtrate was dried (MgSO₄), evaporated and the residue dissolved in ethanol (3 ml) and concentrated hydrochloric acid (0.5 ml) added. Solvent was removed under reduced pressure and the resultant solid washed with ether and dried in vacuo at 40° C. to afford the title compound (234 mg).

NMR (DMSO-d6) 1.45-1.6 (4H, m), 2.78 (2H, q), 3.61 (4H, m). 5.07 (1H, br s), 7.89 (3H, br s).

### Description 17: 6-(2,3-Dichloro-phenylamino)-4-trifluoromethyl-nicotinic acid methyl ester

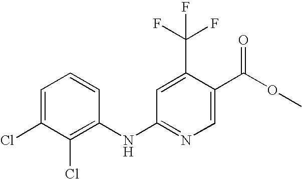

A mixture of methyl-6-chloro-4-(trifluoromethyl)-nicotinate (2.0 g, 8.37 mmol, ex Fluorochem) and 2,3-dichloroaniline (4.06 g, 25 mmol) was heated at 130° C. for 18 h, to afford the title compound.

MS m/z (ESI+): 365, 367 and 369 (isomeric peaks) (MH+).

### Description 18: 6-(2,3-Dichloro-phenylamino)-4-trifluoromethyl-nicotinic acid

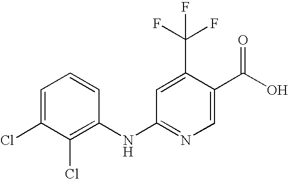

A solution of KOH (1.4 g, 25 mmol) in 20 mL of EtOH/H₂O (1:1) was added to the crude mixture from Description 17 and the resulting mixture was stirred under reflux for 3 h. The solution was concentrated in vacuo, diluted with water and washed three times (3×15 mL) with diethyl ether. Upon acidification of the aqueous layer to pH1 with HCl 37%, the title compound precipitated out as the hydrochloride salt that was filtered and dried under vacuum. The solid (2.7 g, 7 mmol) was then suspended in dichloromethane (20 mL), in the presence of PS-diisopropylethylamine (1.80 g, 7 mmol, loading 3.88 mmol/g, ex Argonaut Technologies) and stirred at room temperature for 30 min. After filtration of the resin and evaporation in vacuo of the solvent, the title compound was isolated as a white solid (2.45 g).

¹H NMR (300 MHz, DMSO-d6) δ: 13.17 (s br, 1H); 9.61 (s, 1H); 8.68 (s, 1H); 7.88 (dd, 1H); 7.44 (dd, 1H); 7.42 (s, 1H); 7.37 (dd, 1H).

MS m/z (ESI+): 351, 353 and 355 (isomeric peaks) (MH+).

### Description 19: 6-(2,4-Dichloro-phenylamino)-4-trifluoromethyl-nicotinic acid methyl ester

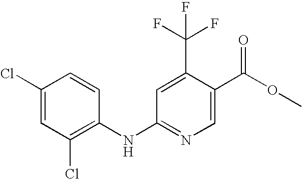

A mixture of methyl-6-chloro-4-(trifluoromethyl)-nicotinate (2.0 g, 8.37 mmol ex Fluorochem) and 2,4-dichloroaniline (4.05 g, 25 mmol) was heated at 130° C. for 15 h, to afford the title compound.

MS m/z (ESI+): 365, 367 and 369 (isomeric peaks) (MH+).

### Description 20: 6-(2,4-Dichloro-phenylamino)-4-trifluoromethyl-nicotinic acid

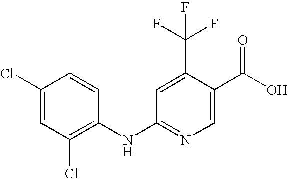

The title compound was prepared in a manner analogous to Description 18 from the product of Description 19 and isolated as a white solid (2.62 g).

¹H NMR (300 MHz, DMSO-d₆) δ: 13.16 (s br, 1H); 9.49 (s, 1H); 8.67 (s, 1H); 7.94 (d, 1H); 7.67 (d, 1H); 7.43 (dd, 1H); 7.40 (s, 1H).

MS m/z (ESI+): 351, 353 and 355 (MH+).

### Description 21: 6-(3-Chloro-phenylamino)-4-trifluoromethyl-nicotinic acid methyl ester

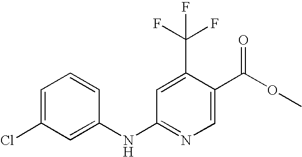

A mixture of methyl-6-chloro-4-(trifluoromethyl)-nicotinate (2.5 g, 10.5 mmol) and 3-chloroaniline (2.2 mL, 20.1 mmol) was heated at 120° C. for 18 h, to afford the title compound.

MS m/z (ESI+): 331 (MH+).

### Description 22: 6-(3-Chloro-phenylamino)-4-trifluoromethyl-nicotinic acid

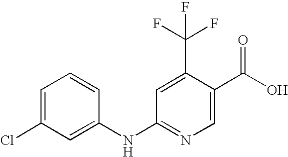

The title compound was prepared in a manner analogous to Description 18 from the product of Description 21 and was isolated as a white solid (1.5 g).

¹H NMR (300 MHz, DMSO-d₆) δ: 13.16 (s br, 1H); 10.28 (s, 1H); 8.80 (s, 1H); 8.01 (dd, 1H); 7.58 (ddd, 1H); 7.35 (dd, 1H); 7.28 (s, 1H); 7.06 (ddd, 1H).

MS m/z (ESI+): 317 (MH+).

### Description 23: 4-Aminomethyl-pyrrolidin-2-one

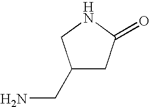

Sodium (0.1 g, 4.34 mmol) was added portionwise to a solution of 4-aminomethyl-1-benzyl-pyrrolidin-2-one (0.3 g, 1.47 mmol, CAS Registry N.: 97205-34-0) in 10 mL of liquid ammonia, at −50° C. and the mixture was stirred at −50° C. for 1 h. EtOH (10 mL) was slowly added and the reaction mixture was allowed to reach room temperature and stirred for 1 h at RT. Evaporation of the solvent in vacuo afforded the title compound (0.21 g), which was used for coupling with the acids above mentioned, without further purification.

¹H NMR (300 MHz, DMSO-d) δ: 3.28 (dd, 1H); 2.89 (dd, 1H); 2.45 (m, 2H); 2.18-1.93 (m, 2H); 1.68 (m, 1H).

### Description 25: 6-Hydroxy-2-trifluoromethyl-4,5-dihydro-pyridine-3-carboxylic acid ethyl ester

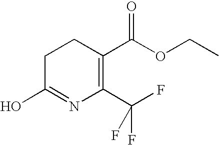

A mixture of ethyl 4,4,4-trifluoroacetoacetate (14.7 mL, 0.1 mol, 1.6 eq), acrylamide (4.5 g, 0.063 mol, 1.0 eq) and p-toluenesulphonic acid (0.156 g, 0.82 mmol, 0.013 eq) in toluene (60 mL) was refluxed for 38 h with azeotropic removal of water (Dean-Stark conditions). The reaction mixture was then concentrated to a small volume, by slow distillation of toluene at atmospheric pressure. Toluene (60 mL) was added and again the reaction mixture was concentrated, through slow distillation of toluene. After repeating this operation three times, the reaction mixture was concentrated in vacuo and the solid residue was purified by flash chromatography (silica gel, eluent gradient: from hexane/ethyl acetate 9:1 to hexane/ethyl acetate 8:2). The title compound was obtained as a brownish solid (3.8 g, yield=25%).

LC-MS (ESI+), MH+: 238, 210, 190.

### Description 26: 6-Hydroxy-2-trifluoromethyl-nicotinic acid ethyl ester

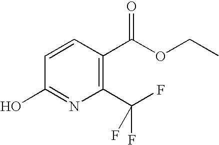

A solution of 6-hydroxy-2-trifluoromethyl-4,5-dihydro-pyridine-3-carboxylic acid ethyl ester (Description 25) (4.7 g, 19.8 mmol, 1 eq) and N-bromo succinimide (3.51 g, 19.8 mmol, 1 eq) in 15 mL of carbon tetrachloride was heated under reflux for 20 h. The resulting precipitate was filtered off and the filtrate was concentrated under reduced pressure to afford a brownish solid that was purified by flash chromatography (silica gel, eluent gradient: from hexane/ethyl acetate 9:1 to hexane/ethyl acetate 8:2). The title compound was obtained as a white solid (4.3 g, yield=92%).

LC-MS (ESI+), MH+: 236.

### Description 27: 6-Chloro-2-trifluoromethyl-nicotinic acid ethyl ester

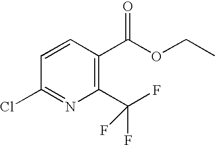

A mixture of 6-hydroxy-2-trifluoromethyl-nicotinic acid ethyl ester (Description 26) (2.6 g, 11.0 mmol, 1.0 eq) and phenyl dichlorophosphate (2.47 mL, 16.5 mmol, 1.5 eq) was heated under microwave irradiation for 30 min (170° C., power=70 W). The reaction mixture was poured into ice, stirred for 20 min and diluted with ethyl acetate (50 mL). The pH was adjusted to 10, by addition of a saturated aqueous solution of sodium bicarbonate (50 mL) and then the organic layer was separated, washed with water, dried over Na₂SO₄ and concentrated in vacuo. The resulting solid residue was purified by flash chromatography (silica gel, eluent gradient: from hexane to hexane/ethyl acetate 98:2) to give 1.7 g of the title compound (yield=61%).

LC-MS (ESI+), MH+: 254 and 256.

### Description 28: 6-(3-Chloro-phenylamino)-2-trifluoromethyl-nicotinic acid ethyl ester

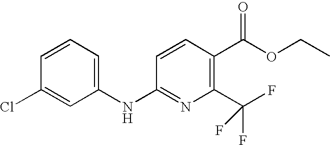

A mixture of 6-chloro-2-trifluoromethyl-nicotinic acid ethyl ester (Description 27) (1.4 g, 5.53 mmol, 1.0 eq) and 3-chloro aniline (2.91 mL, 27.6 mmol, 5.0 eq) was heated at 160° C. for 52 h to afford a black solid which was used for the next step without further purification.

LC-MS (ESI+), MH+: 345 and 347.

### Description 29: 6-(3-Chloro-phenylamino)-2-trifluoromethyl-nicotinic acid hydrochloride

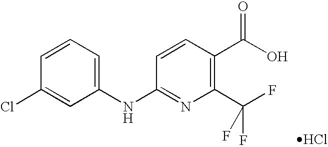

A solution of KOH (1.18 g) in water (25 mL) was added to a mixture of crude 6-(3-chloro-phenylamino)-2-trifluoromethyl-nicotinic acid ethyl ester (Description 28) in ethanol (25 mL) and refluxed for 8 h. After evaporation of ethanol under reduced pressure, the reaction mixture was diluted with water (35 mL) and repeatedly washed with diethyl ether (200 mL×5 times). The aqueous layer was treated with conc. HCl to adjust the pH to 3 and the title compound precipitated out as its hydrochloride salt, was filtered and dried at 40° C. in oven (1.71 g, yield of Description 28 and 29=87%).

LC-MS (ESI+), MH+: 317 and 319.

### Description 30: 3-Amino-4-methyl-pent-2-enoic acid ethyl ester

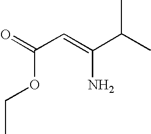

Ammonium acetate (2.44 g, 31.6 mol, 5 eq) was added to a solution of 4-methyl-3-oxo-pentanoic acid ethyl ester (1.0 g, 6.32 mol, 1 eq) in methanol (10 mL) and the mixture was stirred at room temperature for 3 days. Solvent was evaporated in vacuo and the solid residue was triturated with dichloromethane (20 mL) and filtered off. The filtrate was then washed with water and brine, dried over Na₂SO₄ and concentrated in vacuo to afford the title compound as a yellow oil (0.85 g, yield=85%).

### Description 31: 4-(1-Amino-2-methyl-propylidene)-pent-2-enedioic acid 5-ethyl ester 1-methyl ester

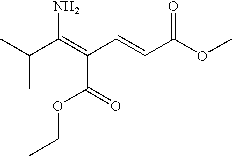

A solution of 3-amino-4-methyl-pent-2-enoic acid ethyl ester (Description 30) (5.0 g, 31.84 mmol, 1 eq) and methyl propiolate (3.08 mL, 36.8 mmol, 1.15 eq) in dry DMSO (20 mL) was heated under microwave irradiation at 170° C. (1^{st} cycle: 20 min, 2^{nd} cycle: 10 min). The reaction mixture was diluted with water (140 mL) and extracted twice with ethyl acetate (80 mL). The organic phase was washed with a saturated aqueous solution of NaHCO₃ and with brine, dried over sodium sulphate and concentrated in vacuo to afford 9.5 g of yellow solid, used for the next step without further purification.

LC-MS (ESI+), MH+: 242, 196.

### Description 32: 6-Hydroxy-2-Isopropyl-nicotinic acid ethyl ester

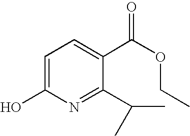

A catalytic amount of sodium tert-butoxide (100 mg) was added to a suspension of crude 4-(1-amino-2-methyl-propylidene)-pent-2-enedioic acid 5-ethyl ester 1-methyl ester (Description 31) (9.5 g) in anhydrous ethanol (100 mL) and the resulting mixture was refluxed for 28 h. Solvent was removed in vacuo, the residue was taken up with ethyl acetate and then washed subsequently with NaHCO₃ (aq) and with brine. The organic layer was dried over Na₂SO₄ and concentrated in vacuo to afford a reddish resin. Trituration of the resin with hexane/diethyl ether 1:1 yielded the title compound as a solid that was filtered off and dried in oven (1.97 g). The mother liquor was concentrated and purified by flash chromatography (silica gel, eluent gradient: from hexane/ethyl acetate 9:1 to hexane/ethyl acetate 7:3) to yield a second crop of pure title compound (1.6 g, total yield of Descriptions 31 and 32=54%).

LC-MS (ESI+), MH+: 210.

### Description 33: 6-Chloro-2-isopropyl-nicotinic acid ethyl ester

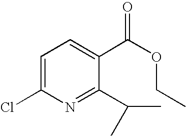

A mixture of 6-hydroxy-2-isopropyl-nicotinic acid ethyl ester (Description 32) 1.0 g, 4.78 mmol, 1.0 eq) and phenyl dichlorophosphate (1.13 mL, 7.56 mmol, 1.5 eq) was heated under microwaves irradiation at 170° C. for 1 min. The reaction mixture was poured into ice-water (25 mL), stirred for 20 min and diluted with ethyl acetate (40 mL). The pH was adjusted to 10, by addition of a saturated aqueous solution of sodium bicarbonate (50 mL) and then the organic layer was separated, washed with water, dried over Na₂SO₄ and concentrated in vacuo to give 1.11 g of the crude title compound as a black resin (yield=99%).

LC-MS (ESI+), MH+: 228 and 230.

### Description 34: 6-(3-Chloro-phenylamino)-2-isopropyl-nicotinic acid ethyl ester

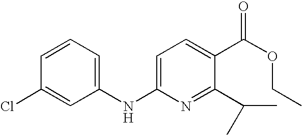

A mixture of 6-chloro-2-isopropyl-nicotinic acid ethyl ester (Description 33) (1.1 g, 4.84 mmol, 1.0 eq) and 3-chloro aniline (1.54 mL, 14.5 mmol, 3.0 eq) was heated at 120° C. for 4 h to afford a solid residue which was used for the next step without further purification.

LC-MS (ESI+), MH+: 319 and 321.

### Description 35: 6-(3-Chloro-phenylamino)-2-isopropyl-nicotinic acid hydrochloride

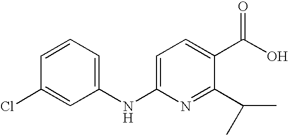

A solution of KOH (1.08 g) in water (10 mL) was added to a mixture of crude 6-(3-chloro-phenylamino)-2-isopropyl-nicotinic acid ethyl ester (Description 34) in ethanol (10 mL) and refluxed for 4 h. After evaporation of ethanol under reduced pressure, the reaction mixture was diluted with water (15 mL) and repeatedly washed with diethyl ether (40 mL×4 times). The aqueous layer was treated with conc. HCl to adjust the pH to 1 and the title compound precipitated out as its hydrochloride salt, was filtered and dried at 40° C. in oven (0.68 g). The aqueous mother liquor was treated with NaCl (s) and repeatedly extracted with ethyl acetate (30 mL×3 times), the organic layer was dried over sodium sulphate and evaporated in vacuo. The residue was treated with conc. HCl and the title compound that precipitated out was filtered and dried in oven (0.681 g, total yield of Description 34 and 35=85%).

LC-MS (ESI+), MH+: 291 and 293.

### Description 36: 6-Chloro-N-(1,1-dioxo-tetrahydro-1⁶-thiophen-3-ylmethyl)-4-isopropyl-nicotinamide

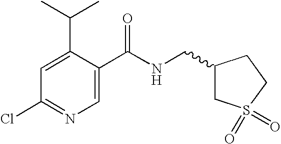

To a solution of 6-chloro-4-isopropyl-nicotinic acid (Description 12) (100 mg) in dimethylformamide (7 ml) was added successively N-ethylmorpholine (0.22 ml), C-(1,1-dioxo-tetrahydro-1⁶-thiophen-3-ylmethyl)-methylamine hydrochloride (111 mg, Ref.: Argyle et al., J. Chem. Soc., (C), 2156, (1967)), 1-hydroxybenzotriazole hydrate (120 mg) and 1-(3-dimethylamino-propyl)-3-ethylcarbodiimide hydrochloride (120 mg). The solution was stirred for 5 h and allowed to stand overnight. Dimethylformamide was removed under reduced pressure and ethyl acetate (20 ml) added. The solution was washed sequentially with 5% sodium bicarbonate solution (12 ml), water (12 ml) and brine (2×12 ml), dried (MgSO₄) and evaporated to afford the title compound (150 mg).

LC/MS t=2.1 min, [MH⁺] 331 consistent with the molecular formula C₁₄H₁₉³⁵ClN₂O₃S.

All the amines used in the Examples are commercially available except as follows where the synthesis of the amines is described in the literature or above.

Amines known in the literature

| Structure | CAS Registry Number |
|---|---|
| 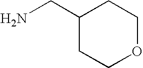 | 130290-79-8 |
| 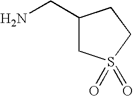 | 45697-13-0 |
| 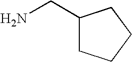 | 6053-81-2 |
| 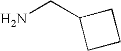 | 4415-83-2 |
| 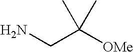 | 89282-70-2 |
| 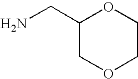 | 88277-83-2 |
| 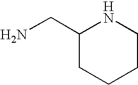 | 22990-77-8 |
| 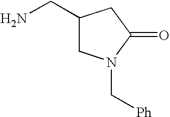 | 97205-34-0 |
| 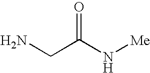 | 22356-89-4 |
| 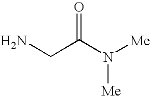 | 1857-19-8 |

### EXAMPLE 1

### 2-(3-Chlorophenylamino)-4-trifluoromethylpyridine-5-carboxylic acid cyclohexylmethyl amide

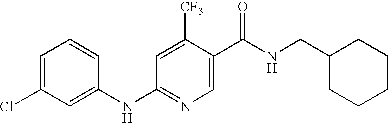

To a solution of 6-(3-chlorophenylamino)-4-(trifluoromethyl)-nicotinic acid hydrochloride (Description 2) (0.2 g) in dimethylformamide (5 mL) were added N-methylmorpholine (283 μL), 4-aminomethylcyclohexane (80 μL), 1-hydroxybenzotriazole hydrate (104 mg), 1-(3-dimethylamino-propyl)-3-ethylcarbodiimide hydrochloride (118 mg). After stirring at room temperature for 6 h, dimethylformamide was evaporated under reduced pressure and dichloromethane added. The solution was washed with a 5% aqueous solution of potassium carbonate (5 mL), then with brine (2×3 mL) and was evaporated under reduced pressure. Chromatographic purification (silica gel; hexane, ethyl acetate 8:2) afforded the title compound (35 mg).

¹H NMR (300 MHz, DMSO-d6) δ 9.85 (1H, s) 8.45 (2H, m), 8.05 (1H, s), 7.5 (1H, d), 7.35 (1H, t), 7.15 (1H, s), 7.02 (1H, d), 3.1 (2H, t), 0.85-1.8 (11H, m).

MS m/z (EI⁺): 411 and 413 (MH⁺.), 328, 315, 299.

IR (KBr): 3412 cm−1, 3309, 2925, 2852, 1648.

### EXAMPLE 2

### 6-(3-Chlorophenylamino)-N-cyclohexylmethyl-4-isopropylnicotinamide

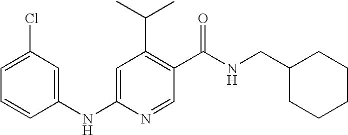

A mixture of 6-chloro-N-cyclohexylmethyl-4-isopropyl-nicotinamide (Description 4) (50 mg) and 3-chloroaniline (90 μl) was heated under microwave conditions at 1900 for 20 minutes. Ethyl acetate (5 ml) was added and the solution washed with dilute potassium carbonate solution (3 ml) and water (3 ml), dried (MgSO₄) and evaporated. The residue was triturated with isohexane to afford the title compound (60 mg).

NMR (DMSO-d6) δ 0.85-1.0 (2H, m), 1.1-1.25 (3H, m), 1.16 (6H, d), 1.51 (1H, m), 1.6-1.8 (5H, m), 3.06 (2H, t), 3.41 (1H, m), 6.78 (1H, s), 6.92 (1H, d), 7.27 (1H, t), 7.46 (1H, d), 8.06 (1H, t), 8.12 (1H, s), 8.33 (1H, t), 9.41 (1H, s).

LC/MS, t=3.7 min, Molecular ion observed [MH⁺]=386 consistent with the molecular formula C₂₂H₂₈ ³⁵ClN₃O.

### EXAMPLE 3

### N-Cyclohexylmethyl-6-(3,4-dichloro-phenylamino)-4-isopropyl-nicotinamide

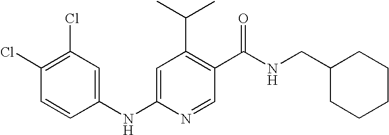

A mixture of 6-chloro-N-cyclohexylmethyl-4-isopropyl-nicotinamide (Description 4) (50 mg), 3,4-dichloroaniline (Aldrich) (33 mg), sodium t-butoxide (46 mg), tris(dibenzylideneacetone)palladium (0) (3.2 mg), 2-(dicyclohexylphosphino)biphenyl (2.6 mg) and dimethoxyethane (1 ml) was irradiated under microwave conditions at 150° for 30 minutes. Solvent was evaporated under reduced pressure and ethyl acetate (5 ml) added. The mixture was washed with water (3 ml), dried (MgSO₄) and evaporated. The residue was purified by mass-directed autopurification techniques to afford the title compound (12.0 mg).

NMR (DMSO-d6) δ 0.85-1.0 (2H, m), 1.1-1.25 (3H, m), 1.16 (6H, d), 1.51 (1H, m), 1.6-1.8 (5H, m), 3.06 (2H, t), 3.41 (1H, m), 6.80 (1H, s), 7.50 (2H, m), 8.13 (1H, s), 8.25 (1H, s), 8.35 (1H, t), 9.62 (1H, s).

LC/MS t=3.9 min, [MH⁺] 420, consistent with molecular formula C₂₂H₂₇³⁵Cl₂N₃O

### EXAMPLE 4

### 6-(3-Bromo-phenylamino)-N-cyclohexylmethyl-4-isopropyl-nicotinamide

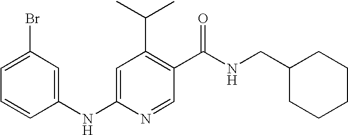

A mixture of 6-chloro-N-cyclohexylmethyl-4-isopropyl-nicotinamide (Description 4) (60 mg) and 3-bromoaniline (Aldrich) (0.5 ml) was irradiated under microwave conditions at 180° for 30 minutes. The mixture was dissolved in dichloromethane and passed down a log SepPak column to remove excess 3-bromoaniline. Elution with 9:1 dichloromethane:ether removed the crude product which was further purified by MDAP to afford the title compound (13.6 mg).

NMR (DMSO-d6) δ 0.85-1.0 (2H, m), 1.1-1.25 (3H, m), 1.17 (6H, d), 1.52 (1H, m), 1.6-1.8 (5H, m), 3.06 (2H, t), 3.42 (1H, m), 6.78 (1H, s), 7.06 (1H, d), 7.22 (1H, t), 7.52 (1H, d), 8.13 (1H, s), 8.19 (1H, s), 8.33 (1H, t), 9.40 (1H, s).

LC/MS t=3.95 min, [MH⁺] 430, consistent with molecular formula C₂₂H₂₈⁷⁹BrN₃0

### EXAMPLE 5

### N-Cyclohexylmethyl-6-(2,4-dichloro-phenylamino)-4-isopropyl-nicotinamide

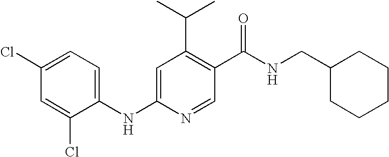

A mixture of 6-chloro-N-cyclohexylmethyl-4-isopropyl-nicotinamide (Description 4) (50 mg), 2,4-dichloroaniline (33 mg), sodium t-butoxide (23 mg), tris(dibenzylideneacetone)palladium(0) (1.6 mg), 2-(dicyclohexylphosphino)biphenyl (1.3 mg) and dimethoxyethane (1 ml) was stirred under reflux for 18 hours. The solvent was evaporated under reduced pressure and ethyl acetate (5 ml) added. The mixture was washed with water (3 ml), dried (MgSO₄) and evaporated. The residue was purified by MDAP to afford the title compound (12 mg).

NMR (DMSO-d6) δ 0.8-1.0 (2H, m), 1.1-1.3 (3H, m), 1.17 (6H, d), 1.50 (1H, m), 1.6-1.8 (5H, m), 3.05 (2H, t), 3.38 (1H, m), 7.08 (1H, s), 7.40 (1H, d), 7.65 (1H, s), 8.01 (1H, s), 8.07 (1H, d), 8.37 (1H, t), 8.93 (1H, br s).

LC/MS t=3.8 min, [MH⁺] 420, consistent with molecular formula C₂₂H₂₇³⁵Cl₂N₃O

### EXAMPLE 6

### 6-(3-Chloro-phenylamino)-N-cyclobutylmethyl-4-isopropyl-nicotinamide

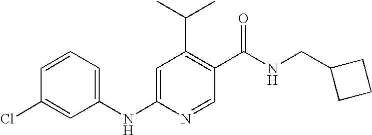

A mixture of 6-chloro-N-cyclobutylmethyl-4-isopropyl-nicotinamide (Description 6) (80 mg) and 3-chloroaniline (0.5 ml) was irradiated under microwave conditions at 180° C. for 30 mins. The mixture was diluted with dichloromethane (2 ml) and chromatographed on silica gel. The excess aniline was removed by elution with dichloromethane and then elution with dichloromethane/ether (5:1) gave the title compound (38 mg).

NMR (DMSO-d6) δ 1.16 (6H, m), 1.74 (2H, m), 1.82 (2H, m), 2.00 (2H, m), 2.52 (1H, m excess), 3.23 (2H, t), 3.40 (1H, m), 6.78 (1H, s), 6.92 (1H, d), 7.27 (1H, t), 7.46 (1H, d), 8.04 (1H, s), 8.10 (1H, s), 8.33 (1H, t), 9.41 (1H, s)

LC/MS t=3.65 min, [MH⁺] 358 consistent with the molecular formula C₂₀H₂₄³⁵ClN₃O

TABLE 1

| | | | 1. | | |
|---|---|---|---|---|---|
| | | | Preparation | | |
| | | | method | | |
| | | | A, B, or C | | 1. Retention |
| | | | 2. Reaction | | Time (min). |
| | | | temperature | Purification | 2. [MH⁺] |
| Ex. | | | (° C.), | method | 3. Molecular |
| No | Chemical Name | Structure | 3. Time. | E or F | formula |
| 7 | 6-(3-Chloro-phenyl- amino)-4-isopropyl-N- (tetrahydro-pyran-4- ylmethyl)-nicotinamide | 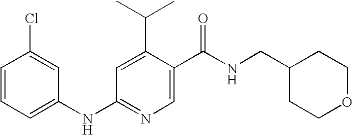 | A 200°1 hr | E | 3.1 388 C₂₁H₂₆³⁵ClN₃O₂ |
| 8 | 6-(3-Bromo-phenyl- amino)-4-isopropyl-N- (tetrahydro-pyran-4- ylmethyl)-nicotinamide | 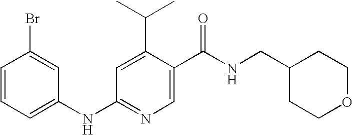 | A 200°30 min | E | 3.1 432 C₂₁H₂₆⁷⁹BrN₃O₂ |
| 9 | N-Cyclohexylmethyl-4- isopropyl-6-(3- methoxy- phenylamino)- nicotinamide | 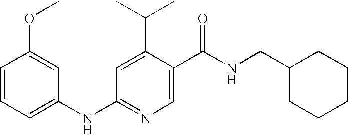 | B 150°30 min | E | 3.4 382 C₂₃H₃₁N₃O₂ |
| 10 | N-Cyclohexylmethyl-6- (3-fluoro- phenylamino)-4- isopropyl-nicotinamide | 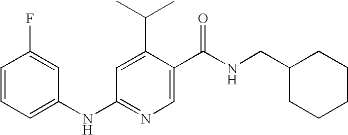 | B 150°30 min | E | 3.6 370 C₂₂H₂₈FN₃O |
| 11 | 1-[6-(3-Chloro- phenylamino)-4- isopropyl-pyridin-3-yl]- 1-morpholin-4-yl- methanone | 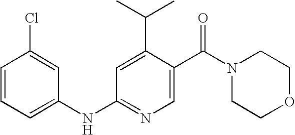 | A 180°30 min | E | 3.1 360 C₁₉H₂₂³⁵ClN₃O₂ |
| 12 | 6-(3-Bromo- cyclohexylmethyl-4- phenylamino)-N- isopropyl-nicotinamide | 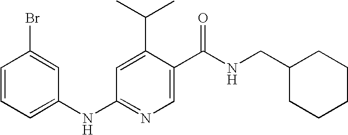 | A 180°30 min | E | 3.95 430 C₂₂H₂₈⁷⁹BrN₃O |
| 13 | N-Cyclohexylmethyl-4- isopropyl-6-m- tolytamino- nicotinamide | 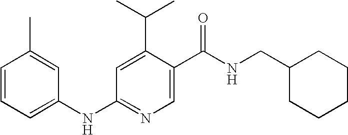 | A 180°(1 hr) | E | 3.68 366 C₂₃H₃₁N₃O |
| 14 | N-Cyclohexylmethyl-4- isopropyl-6-(3- trifluoromethyl- phenylamino)- nicotinamide | 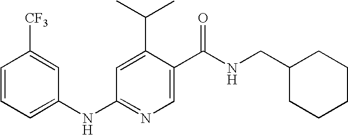 | A 180°1 hr | E | 3.7 420 C₂₃H₂₈F₃N₃O |
| 15 | N-Cyclohexylmethyl-4- isopropyl-6-(3- trifluoromethoxy- phenylamino)- nicotinamide | 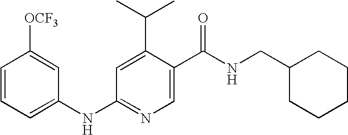 | A 180°30 min | E | 3.8 436 C₂₃H₂₈F₃N₃O₂ |
| 16 | 6-(2,3-Dichloro- phenylamino)-4- isopropyl-N- (tetrahydro-pyran-4- ylmethyl)-nicotinamide | 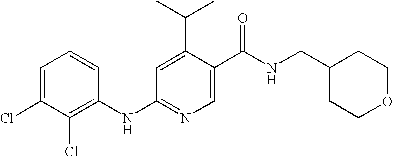 | B 150°30 min | E | 3.34 422 C₂₁H₂₅³⁵Cl₂N₃O₂ |
| 17 | 6-(2,4-Dichloro- phenylamino)-4- isopropyl-N- (tetrahydro-pyran-4- ylmethyl)-nicotinamide | 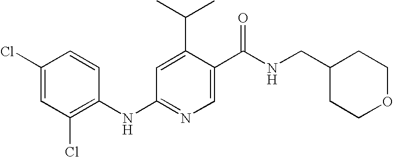 | B 150°30 min | E | 3.39 422 C₂₁H₂₅³⁵Cl₂N₃O₂ |
| 18 | 6-(3,4-Dichloro- phenylamino)-4- isopropyl-N- (tetrahydro-pyran-4- ylmethyl)-nicotinamide | 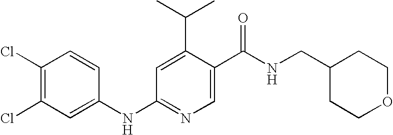 | B 150°30 min | E | 3.51 422 C₂₁H₂₅³⁵Cl₂N₃O₂ |
| 19 | 4-Isopropyl-N- (tetrahydro-pyran-4- ylmethyl)-6-(3- trifluoromethyl- phenylamino) nicotinamide | 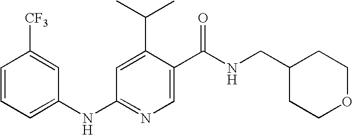 | A 180°1 hr | E | 3.2 422 C₂₂H₂₆F₃N₃O₂ |
| 20 | 4-Isopropyl-N- (tetrahydro-pyran-4- ylmethyl)-6-(3- trifluoromethoxy- phenylamino) nicotinamide | 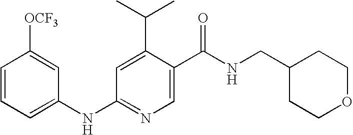 | A 180°30 min | E | 3.3 438 C₂₂H₂₆F₃N₃O₃ |
| 21 | 6-[(3-Chloro- phenyl)amino]-N- (cyclopentylmethyl)-4- isopropyl-nicotinamide | 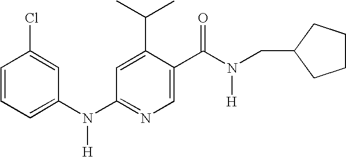 | A 180°30 min | E | 3.76 372 C₂₁H₂₆N₃ClO |
| 22 | N-Cyclopentylmethyl-6- (3-fluorophenylamino)- 4-isopropyl- nicotinamide | 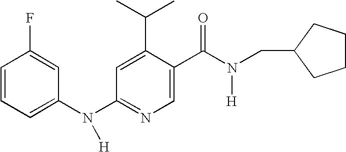 | A 180°30 min | E | 3.69 356 C₂₁H₂₆N₃FO |
| 23 | N-Cyclopentylmethyl-4- isopropyl-6-(3- trifluoromethyl- phenylamino)- nicotinamide | 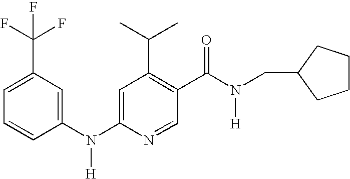 | A 180°30 min | E | 3.82 406 C₂₁H₂₆N₃F₃O |
| 24 | N-Cyclopentylmethyl-4- isopropyl-6-m- tolylamino-nicotinamide | 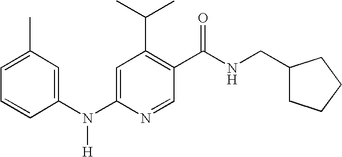 | A 180°30 min | E | 3.52 352 C₂₂H₂₉ON₃ |
| 25 | N-Cyclopentylmethyl-4- isopropyl-6-(3- trifluoromethoxy- phenylamino)- nicotinamide | 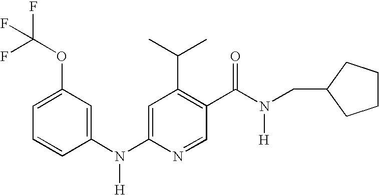 | A 180°30 min | E | 3.86 422 C₂₂H₂₆N₃O₂F₃ |
| 26 | 6-(3- Bromophenylamino)-N- cyclopentylmethyl-4- isopropyl-nicotinamide | 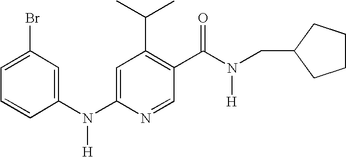 | A 180°30 min | E | 3.86 422 C₂₁H₂₆N₃OBr |
| 27 | N-Cyctopentylmethyl-4- isopropyl-6-(3- methoxyphenylamino) nicotinamide | 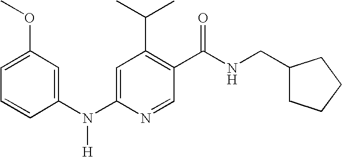 | A 180°30 min | E | 3.81 418 C₂₂H₂₉N₃O₂ |
| 28 | 6-(3-Cyano- phenylamino)-N- cyclopentylmethyl-4- isopropyl-nicotinamide | 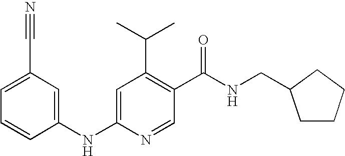 | A 180°30 min | E | 3.55 363 C₂₂H₂₈N₄O |
| 29 | 6-(2-Chloro-4-fluoro- phenylamino)-N- cyclopentylmethyl-4- isopropyl-nicotinamide | 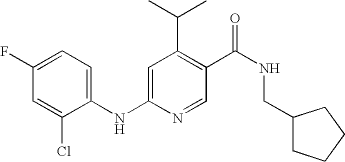 | A 180°30 min | E | 3.6 391 C₂₁H₂₅N₃ClFO |
| 30 | 6-(2-Chloro-4-cyano- phenylamino)-N- cyclopentylmethyl-4- isopropyl-nicotinamide | 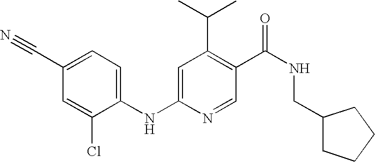 | A 180°30 min | E | 3.76 398 C₂₂H₂₅N₄ClO |
| 31 | N-Cyclopentylmethyl-6- (2,4-dichloro- isopropyl-nicotinamide | 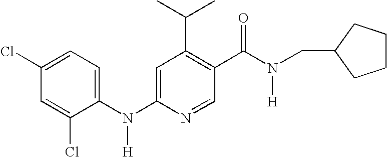 | A 180°30 min | E | 3.70 407 C₂₁H₂₅N₃Cl₂O |
| 32 | N-Cyclopentylmethyl-6- (3,4- dichlorophenyl)amino)- 4-isopropyl- nicotinamide | 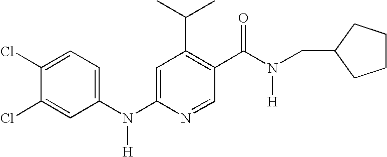 | A 180°30 min | E | 3.80 407 C₂₁H₂₅N₃Cl₂O |
| 33 | 6-(3-Bromo- phenylamino)-N- cyclobutylmethyl-4- isopropyl-nicotinamide | 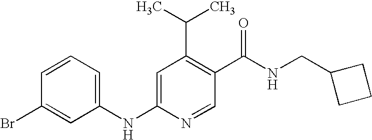 | C 180°30 min | F | 3.70 402 C₂₀H₂₄⁷⁹BrN₃O |
| 34 | N-Cyclobutylmethyl-6- (3-fluoro- phenylamino)-4- isopropyl-nicotinamide | 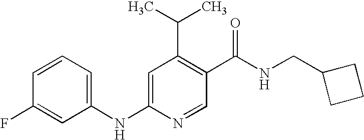 | C 180°30 min | F | 3.49 342 C₂₀H₂₄FN₃O |
| 35 | N-Cyclobutylmethyl-6- (3-trifluoromethyl- phenylamino)-4- | 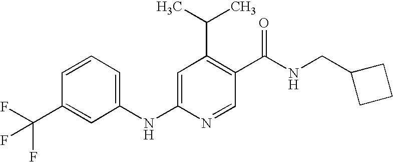 | C 180°30 min | F | 3.53 392 C₂₁H₂₄F₃N₃O |
| 36 | 6-(3-Cyano- phenylamino)-N- cyclobutylmethyl-4- isopropyl-nicotinamide | 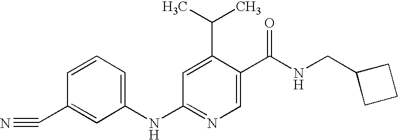 | C 180°30 min | F | 3.41 349 C₂₁H₂₄N₄O |
| 37 | N-Cyclobutylmethyl-4- isopropyl-6-m- tolylamino- nicotinamide | 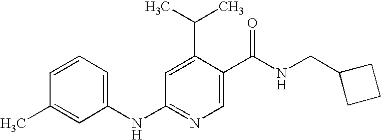 | C 180°1 hr | F | 3.39 338 C₂₁H₂₇N₃O |
| 38 | N-Cyclobutylmethyl-4- isopropyl-6-(3- methoxy- phenylamino)- nicotinamide | 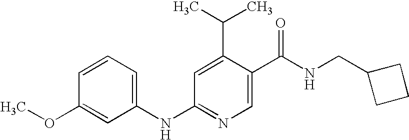 | C 180°1 hr | F | 3.30 354 C₂₁H₂₇N₃O₂ |

Preparative Method A: As for Example 2, with temperature and time of reaction, and any other variations included in the table.
Preparative Method B: As for Example 3, with temperature and time of reaction, and any other variations noted in the table.
Preparative Method C: As for Example 6, with temperature and time of reaction, and any other variations noted in the table.
Purification Method E: Purify by mass-directed autopurification techniques.
Purification Method F: The crude product was diluted with dichloromethane (2 ml) and the solution applied to a Sep-Pack column of silica gel. This was eluted firstly with dichloromethane, followed by dichloromethane/ether 5:1 to give pure product.

TABLE 2
The Examples 39 to 45 in Table 2 were prepared in a manner similar to as Example 2 with the
reaction temperature and time given in the table. An asterisk in the fourth column signifies that
the preparative method used was the same as that used in Example 46 and the product was
purified by the method given in the 5th column.

| | | | | | 1. Retention |
|---|---|---|---|---|---|
| | | | 1. Reaction | | Time (min). |
| | | | Temperature | Purification | 2. [MH⁺] |
| Ex. | | | 2. Reaction | method | 3. Molecular |
| No | Name | Structure | Time | E, or F | formula |
| 39 | 6-(3-Fluoro- phenylamino)-4- isopropyl-N- (tetrahydro-pyran-4- ylmethyl)-nicotinamide | 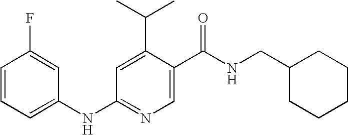 | 200°1 hr | E | 2.9 372 C₂₁H₂₆FN₃O₂ |
| 40 | 1-[6-(3-Fluoro- phenylamino)-4- isopropyl-pyridin-3-yl]- 1-morpholin-4-yl- methanone | 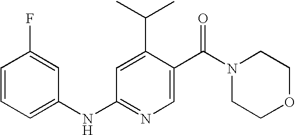 | 180°30 min | E | 2.9 344 C₁₉H₂₂FN₃O₂ |
| 41 | 4-Isopropyl-6-(3- methoxy-phenylamino)- N-(tetrahydro-pyran-4- ylmethyl)-nicotinamide | 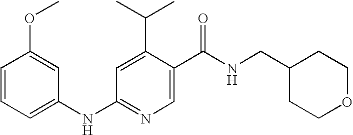 | 180°2 hr | E | 2.7 384 C₂₂H₂₉N₃O₃ |
| 42 | 4-lsopropyl-N- (tetrahydro-pyran-4- ylmethyl)-6-m- tolylamino-nicotinamide | 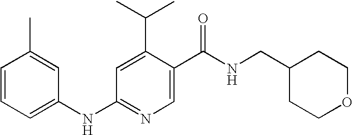 | 180°1 hr | E | 2.93 368 C₂₂H₂₉N₃O₂ |
| 43 | 6-(3-Cyano- phenylamino)-4- isopropyl-N- (tetrahydro-pyran-4- ylmethyl)-nicotinamide | 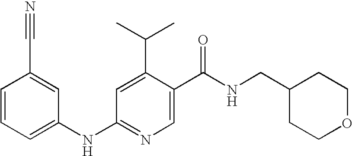 | 180°30 min | E | 2.8 379 C₂₂H₂₈N₄O₃ |
| 44 | 6-[(3,4-Dichloro- phenyl)-methyl-amino]- 4-isopropyl-N- (tetrahydro-pyran-4- ylmethyl)-nicotinamide | 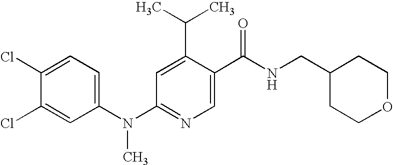 | 180°2 hrs * | E | 3.51 436 C₂₂H₂₇³⁵Cl₂N₃O₂ |
| 45 | 6-[(3-Bromo-phenyl)- methyl-amino]-4- isopropyl-N- (tetrahydro-pyran-4- ylmethyl)-nicotinamide | 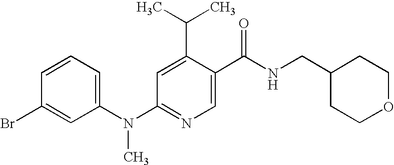 | 180°2 hrs * | F | 3.31 446 C₂₂H₂₈⁷⁹BrN₃O₂ |

Purification Method E: Purify by mass-directed autopurification techniques.
Purification Method F: The crude product was diluted with dichloromethane (2 ml) and the solution applied to a Sep-Pack column of silica gel. This was eluted firstly with dichloromethane, followed by dichloromethane/ether 5:1 to give pure product.

### EXAMPLE 46

### 6-[(3-Fluoro-phenyl)-methyl-amino]-4-isopropyl-N-(tetrahydro-pyran-4-ylmethyl)-nicotinamide

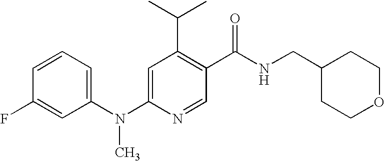

A mixture of 6-chloro-4-isopropyl-N-(tetrahydro-pyran-4-ylmethyl)-nicotinamide (Description 8) (89 mg), 3-fluoro-N-methylaniline (75 mg) and methanesulphonic acid (72 mg) in dioxan (1 ml) was heated on microwave at 180° C. for 2 hours. The mixture was diluted with ethyl acetate (20 ml) and washed with sodium bicarbonate solution (20 ml) and water (2×20 ml) and evaporated to an oil. Purification by chromatography on silica gel (dichloromethane then dichloromethane/methanol 10:1) gave a solid which was triturated with ether/isohexane 1:1 to give the title compound (63 mg).

NMR (DMSO-d6) δ 1.05 (6H, d), 1.15 (2H, m), 1.60 (2H, d), 1.74 (1H, m), 3.10 (2H, t), 3.26 (2H, m), 3.34 (1H, m excess), 3.42 (3H, s), 3.84 (2H, m), 6.64 (1H, s), 7.02 (1H, m), 7.14 (2H, m), 7.43 (1H, q), 8.11 (1H, s), 8.35 (1H, t).

LC/MS t=2.97 min, Molecular ion observed [MH⁺]=386 consistent with the molecular formula C₂₂H₂₈FN₃O₂

TABLE 3
All examples prepared in Table 3 were prepared by the same method as given for
Example 46, with variations in reaction time, and purification method given in the table.

| | | | | | 1. Retention Time |
|---|---|---|---|---|---|
| | | | | | (min). |
| | | | | | 2.[MH⁺] |
| Ex. | | | Reaction | Purification, E | 3. Molecular |
| No | Compound Name | Compound Structure | time | or F | formula |
| 47 | 4-lsopropyl-6- (methyl-phenyl- amino)-N- (tetrahydro-pyran 4-ylmethyl)- nicotinamide | 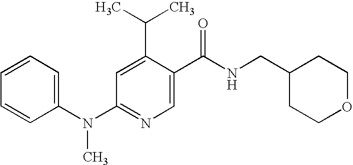 | 1 hr | E, then silica gel chromatography CH₂Cl₂:MeOH, 50:1, then 25:1 | 2.67 368 C₂₂H₂₉N₃O₂ |
| 48 | 6-[(3-Chloro- phenyl)-methyl- amino]-4- isopropyl-N- (tetrahydro-pyran- 4-ylmethyl)- nicotinamide | 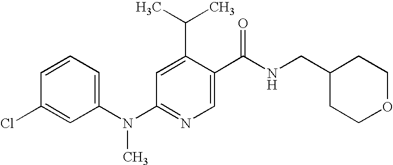 | 2 hrs | E | 3.22 402 C₂₂H₂₈³⁵ClN₃O₂ |
| 49 | 6-[(4-Chloro- phenyl)-methyl- amino]-4- isopropyl-N- (tetrahydro-pyran- 4-ylmethyl)- nicotinamide | 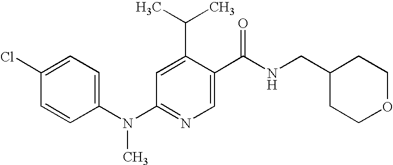 | 2 hrs | E | 3.20 402 C₂₂H₂₈³⁵ClN₃O₂ |

Purification Method E: Purify by mass-directed autopurification techniques.
Purification Method F: The crude product was diluted with dichloromethane (2 ml) and the solution applied to a Sep-Pack column of silica gel. This was eluted firstly with dichloromethane, followed by dichloromethane/methanol 10:1 to give pure product.

### EXAMPLE 50

### 6-(3-Chloro-phenylamino)-N-cyclobutyl-4-isopropyl-nicotinamide

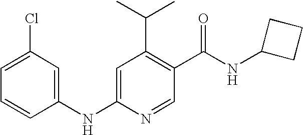

To a solution of 6-(3-chloro-phenylamino)-4-isopropyl-nicotinic acid (Description 13) (48 mg) in dimethylformamide (2.5 ml) was added successively N-ethylmorpholine (69 μl), cyclobutylamine (17 μl), 1-hydroxybenzotriazole hydrate (40 mg) and 1-(3-dimethylamino-propyl)-3-ethylcarbodiimide hydrochloride (40 mg). The solution was stirred for 3 hours and allowed to stand overnight. Dimethylformamide was removed under reduced pressure and ethyl acetate (8 ml) added. The solution was washed sequentially with 5% sodium bicarbonate solution (5 ml), water (5 ml) and brine (2×5 ml), dried (MgSO₄) and evaporated to afford the title compound (40 mg).

NMR (DMSO-d6) δ 1.16 (6H, d), 1.65 (2H, m), 1.99 (2H, m), 2.2 (2H, m), 3.40 (1H, m), 4.35 (1H, m), 6.77 (1H, s), 6.92 (1H, d), 7.28 (1H, t), 7.46 (1H, d), 8.06 (1H, t), 8.13 (1H, s), 8.56 (1H, d), 9.42 (1H, s).

LC/MS t=3.51 min, [MH⁺] 344, consistent with molecular formula C₁₉H₂₂³⁵ClN₃O

The compounds in Tables 4, 5, and 6 were synthesized by the method used to prepare Example 50.

TABLE 4

| | | | 1. Retention Time (min) |
|---|---|---|---|
| Ex. | | | 2. [MH⁺] |
| No | Name | Structure | 3. Molecular formula |
| 51 | 6-(3-Chloro- phenylamino)-N- cyclopropylmethyl-4- isopropyl-nicotinamide | 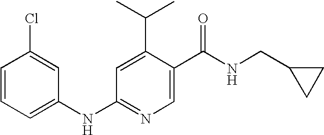 | 3.47 344 C₁₉H₂₂³⁵ClN₃O |
| 52 | 6-(3-Chloro- phenylamino)-N-(2- ethyl-butyl)-4-isopropyl- nicotinamide | 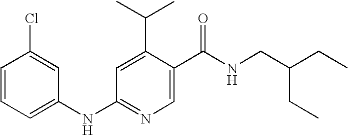 | 3.8 374 C₂₁H₂₈³⁵ClN₃O |
| 53 | 6-(3-Chloro- Phenylamino)-N- cyclohexyl-4-isopropyl- nicotinamide | 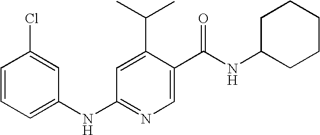 | 3.7 372 C₂₁H₂₆³⁵ClN₃O |
| 54 | 6-(3-Chloro- phenylamino)-N-(1- hydroxy- cyclohexylmethyl)-4- isopropyl-nicotinamide | 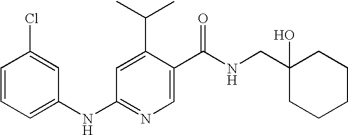 | 3.46 402 C₂₂H₂₈³⁵ClN₃O₂ |
| 55 | 1-[6-(3-Chloro- phenylamino)-4- isopropyl-pyridin-3-yl]- 1-piperidin-1-yl- methanone | 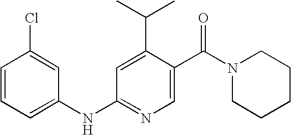 | 3.57 358 C₂₀H₂₄³⁵ClN₃O |

TABLE 5

| | | | 1. Retention Time |
|---|---|---|---|
| | | | (min). |
| Ex. | | | 2. [MH⁺] |
| No | Name | Structure | 3. Molecular formula |
| 56 | 6-(3-Chloro- phenylamino)-N-(2,2- dimethyl-propyl)-4- isopropyl-nicotinamide | 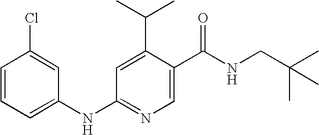 | 3.6 360 C₂₀H₂₆³⁵ClN₃O |
| 57 | 6-(3-Chloro- phenylamino)-4- isopropyl-N-(2- methoxy-ethyl)- nicotinamide | 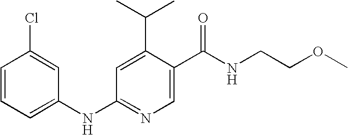 | 3.0 348 C₁₈H₂₂³⁵ClN₃O₂ |
| 58 | 6-(3-Chloro- phenylamino)-4- isopropyl-N- (tetrahydro-pyran-4-yl)- nicotinamide | 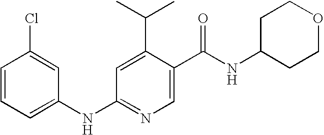 | 3.0 374 C₂₀H₂₄³⁵ClN₃O₂ |
| 59 | 6-(3-Chloro- phenylamino)-4- isopropyl-N-[(R)-1- (tetrahydro-furan-2- yl)methyl]-nicotinamide | 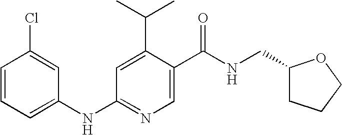 | 3.30 374 C₂₀H₂₄³⁵ClN₃O₂ |
| 60 | N-((R)-1-{1-[6-(3- Chloro-phenylamino)- 4-isopropyl-pyridin-3- yl]-methanoyl}- pyrrolidin-3-yl)- acetamide | 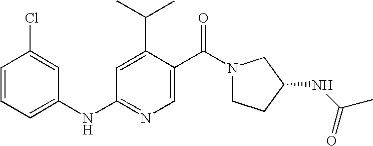 | 2.77 401 C₂₁H₂₅³⁵ClN₄O₂ |
| 61 | 1-[6-(3-Chloro- phenylamino)-4- 1-(4-methane-sulfonyl- piperazin-1-yl)- methanone | 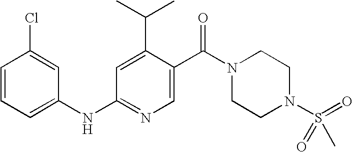 | 3.1 437 C₂₀H₂₅³⁵ClN₄O₃S |
| 62 | 6-(3-Chloro- phenylamino)-N-(1,1- dioxo-tetrahydro-1/⁶- thiophen-3-yl)-4- isopropyl-nicotinamide | 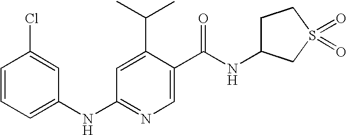 | 3.0 408 C₁₉H₂₂³⁵ClN₃O₃S |

TABLE 6

| | | | 1. Retention Time |
|---|---|---|---|
| | | | (mm). |
| Ex. | | | 2. [MH⁺] |
| No | Name | Structure | 3. Molecular formula |
| 63 | 6-(3-Chloro- phenylamino)-4- isopropyl-N-[(S)-1- (tetrahydro-furan-2- yl)methyl]-nicotinamide | 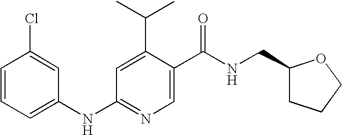 | 3.30 374 C₂₀H₂₄³⁵ClN₃O₂ |
| 64 | 6-(3-Chloro- phenylamino)-N-(1,1- dioxo-hexahydro-1/⁶- thiopyran-4-yl)-4- isopropyl-nicotinamide | 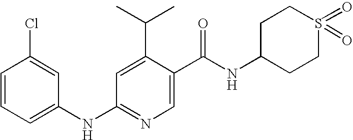 | 2.9 422 C₂₀H₂₄³⁵ClN₃O₃S |
| 65 | 1-[6-(3-Chloro- phenylamino)-4- isopropyl-pyridin-3-yl]- 1-(4-methyl-piperazin- 1-yl)-methanone | 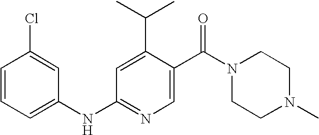 | 2.18 373 C₂₀H₂₅³⁵ClN₄O |
| 66 | 6-(3-Chloro- phenylamino)-N-(2- dimethylamino-ethyl)-4- isopropyl-nicotinamide | 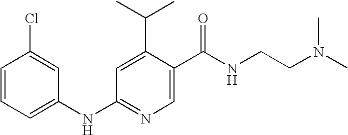 | 2.20 361 C₁₉H₂₅³⁵ClN₄O |
| 67 | N-((S)-1-{1-[6-(3- Chloro-phenylamino)- yl]-methanoyl}- pyrrolidin-3-yl)- acetamide | 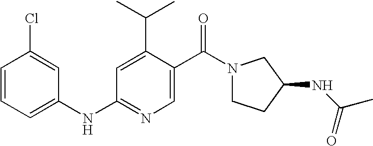 | 2.77 401 C₂₁H₂₅³⁵ClN₄O₂ |
| 68 | N-(1-{1-[6-(3-Chloro- phenylamino)-4- isopropyl-pyridin-3-yl]- methanoyl}-piperidin-4- yl)- methanesulfonamide | 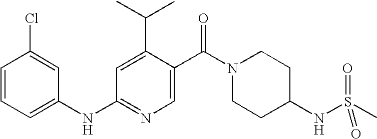 | 2.9 451 C₂₁H₂₇³⁵ClN₄O₃S |

### EXAMPLE 69

### 4-tert-Butyl-6-(3-chloro-phenylamino)-N-cyclohexylmethyl-nicotinamide

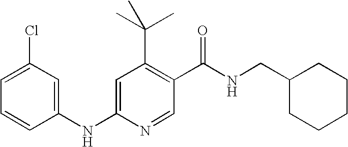

A solution of 4-tert-butyl-6-chloro-N-cyclohexylmethyl-nicotinamide (Description 14) (41 mg), 3-chloroaniline (21 μl) and methanesulphonic acid (17 μl) in dioxan (0.5 ml) was irradiated under microwave conditions at 180° for 30 minutes. Solvent was evaporated under reduced pressure and the residue purified by MDAP to afford the title compound (35 mg).

NMR (DMSO-d6) δ 0.85-1.0 (2H, m), 1.1-1.25 (3H, m), 1.35 (9H, s), 1.55 (1H, m), 1.6-1.8 (5H, m), 3.03 (2H, t), 6.87 (1H, s), 6.92 (1H, d), 7.27 (1H, t), 7.46 (1H, d), 7.95 (1H, s), 8.03 (1H, t), 8.36 (1H, t), 9.39 (1H, s).

LC/MS t=4.20 min, [MH⁺] consistent with molecular formula C₂₃H₃₀³⁵ClN₃O

TABLE 7
The compounds prepared in Table 7 were prepared in a manner similar to Example 69
from the intermediates in Description 14 or Description 15, with the reaction time given in
Table 7.

| | | | | 1. Retention Time |
|---|---|---|---|---|
| | | | | (mm). |
| | | | Reaction | 2. [MH⁺] |
| Ex. | | | time | 3. Molecular |
| No | Name | Structure | (minutes) | formula |
| 70 | 4-tert-Butyl-6-(2,4- dichloro- phenylamino)-N- cyclohexylmethyl- nicotinamide | 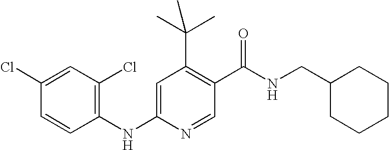 | 75 | 4.35 434 C₂₃H₂₉³⁵Cl₂N₃O |
| 71 | 4-tert-Butyl-6-(3- phenylamino)-N- (tetrahydro-pyran-4- ylmethyl)- nicotinamide | 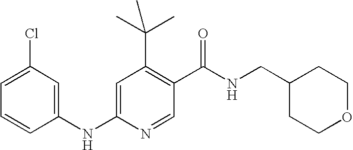 | 30 | 3.40 402 C₂₂H₂₈³⁵ClN₃O₂ |
| 72 | 4-tert-Butyl-6-(3- fluoro-phenylamino)- N-(tetrahydro-pyran- 4-ylmethyl)- nicotinamide | 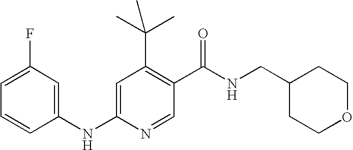 | 30 | 3.21 386 C₂₂H₂₈FN₃O₂ |
| 73 | 4-tert-Butyl-6-(2- chloro-3- fluorophenylamino)- N-(tetrahydro-pyran- 4-ylmethyl)- nicotinamide | 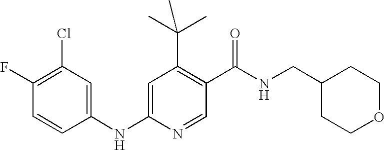 | 30 | 3.40 420 C₂₂H₂₇³⁵ClFN₃O₂ |
| 74 | 4-tert-Butyl-6-(2,4- di-chloro- phenylamino)-N- (tetrahydro-pyran-4- ylmethyl)- nicotinamide | 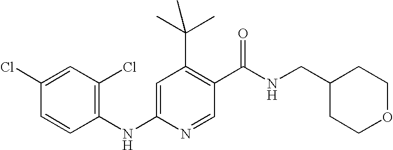 | 60 | 3.40 436 C₂₂H₂₇³⁵Cl₂N₃O₂ |

### EXAMPLE 75

### 6-(3,5-Dichloro-phenylamino)-4-isopropyl-N-(tetrahydro-pyran-4-ylmethyl)-nicotinamide

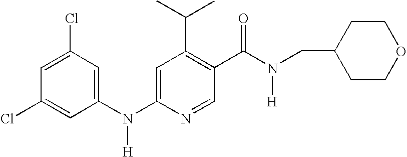

A mixture of 6-chloro-4-isopropyl-N-(tetrahydro-pyran-4-ylmethyl)-nicotinamide (Description 8) (100 mg), 3,5-dichloroaniline (ex-Aldrich, 109 mg), methanesulfonic acid (44 μl) in 1,4-dioxane (1 ml) was irradiated under microwave conditions at 180° C. for 30 minutes, The crude mixture was purified using MDAP to afford 6-(3,5-dichloro-phenylamino)-4-isopropyl-N-(tetrahydro-pyran-4-ylmethyl)-nicotinamide (50 mg) NMR (CDCl₃) δ1.21-1.29 (6H, m), 1.35-1.48 (2H, m), 1.35-1.49 (2H, m), 1.71 (2H, d), 1.86-1.99 (1H, m), 3.34-3.49 (4H, m), 3.50-3.61 (1H, m), 4.03 (2H, d), 6.10 (1H, bs), 6.75 (1H, bs), 7.08 (1H, bs), 7.10-7.16 (1H, m), 7.41-7.45 (2H, m), 8.26 (1H, s)

TABLE 8

| | | | | | RT (min), |
|---|---|---|---|---|---|
| | | | | | (MH⁺), |
| | | | | | Consistent |
| | | | | | with the |
| | | | | Purification | molecular |
| | Chemical Name | Structure | Method | method | formula |
| 76 | 6-(5-Chloro-2-fluoro- phenylamino)-4- isopropyl-N- (tetrahydro-pyran-4- ylmethyl)- nicotinamide | 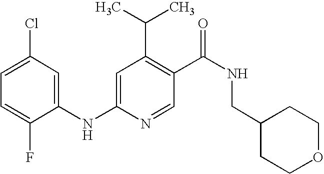 | G | E | 3.13 406 C₂₁H₂₅³⁵ClFN₃O₂ |
| 77 | 6-(3-Chloro-4-fluoro- phenylamino)-4- isopropyl-N- (tetrahydro-pyran-4- ylmethyl)- nicotinamide | 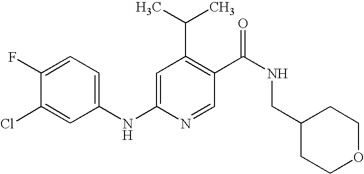 | G | E | 3.13 406 C₂₁H₂₅³⁵ClFN₃O₂ |
| 78 | 6-(3-Chloro-4- trifluoromethoxy- phenylamino)-4- isopropyl-N- (tetrahydro-pyran-4- ylmethyl)- nicotinamide | 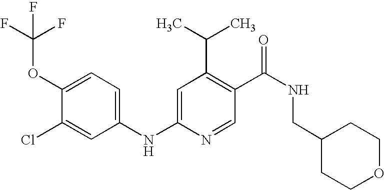 | G | E | 3.62 472 C₂₂H₂₅³⁵ClF₃N₃O₃ |
| 79 | 6-(3-Chloro-4- cyano- phenylamino)-4- isopropyl-N- (tetrahydro-pyran-4- ylmethyl) nicotinamide | 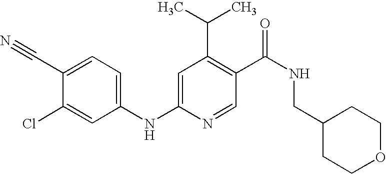 | G | E | 3.10 413 C₂₂H₂₅³⁵ClN₄O₂ |
| 80 | 6-(3-Fluoro-5- trifluoromethyl- phenylamino)-4- isopropyl-N- (tetrahydro-pyran-4- ylmethyl) nicotinamide | 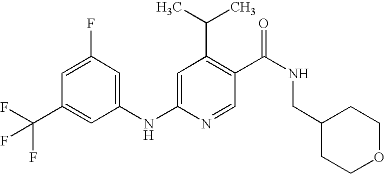 | G | E | 3.20 440 C₂₂H₂₅F₄N₃O₂ |
| 81 | 6-(2-Fluoro-3- trifluoromethyl- isopropyl-N- phenylamino)-4- (tetrahydro-pyran-4- ylmethyl) nicotinamide | 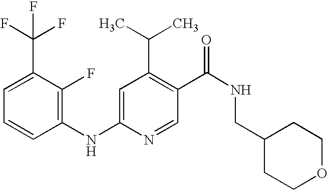 | G | E | 3.40 440 C₂₂H₂₅F₄N₃O₂ |
| 82 | 6-(4-Bromo-2- chloro- phenylamino)-4- isopropyl-N- ylmethyl)- nicotinamide | 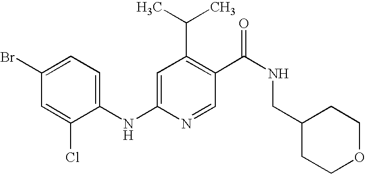 | G | E | 3.41 468 C₂₁H₂₅⁷⁹Br³⁵ClN₃O₂ |
| 83 | 6-(2-Bromo-4- chloro- phenylamino)-4- isopropyl-N- (tetrahydro-pyran-4- ylmethyl) nicotinamide | 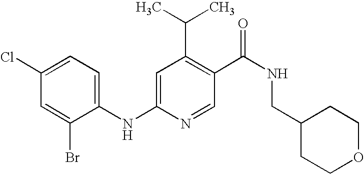 | G | E | 3.39 468 C₂₁H₂₅⁷⁹Br³⁵ClN₃O₂ |
| 84 | 4-lsopropyl-6-(2- methyl-3- trifluoromethyl- phenylamino)-N- (tetrahydro-pyran-4- ylmethyl)- nicotinamide | 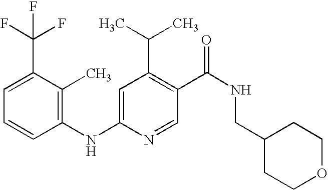 | G | E | 3.09 436 C₂₃H₂₈F₃N₃O₂ |
| 85 | 6-(3-chloro-4- methyl- phenylamino)-4- (tetrahydro-pyran-4- ylmethyl)- nicotinamide | 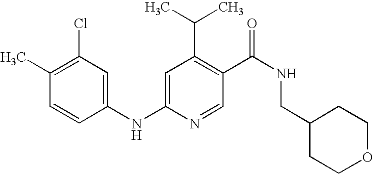 | G | H | 3.24 402 C₂₂H₂₈³⁵ClN₃O₂ |
| 86 | 6-(4-Bromo-3- methyl- phenylamino)-4- isopropyl-N- (tetrahydro-pyran-4- ylmethyl)- nicotinamide | 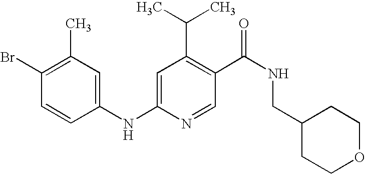 | G | A | 2.48 446 C₂₂H₂₈⁷⁹BrN₃O₂ |
| 87 | 6-(2,5-Dichloro- phenylamino)-4- isopropyl-N- (tetrahydro-pyran-4- ylmethyl)- nicotinamide | 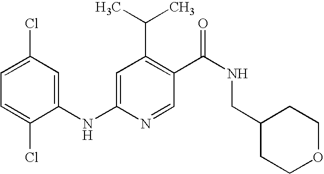 | G NB Irradi- ation time was 60 min. | E | 3.28 422 C₂₁H₂₅³⁵Cl₂N₃O₂ |
| 88 | 4-lsopropyl-6-(2- methyl-5- trifluoromethyl- phenylamino)-N- (tetrahydro-pyran-4- ylmethyl)- nicotinamide | 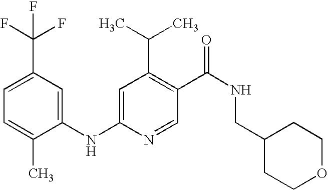 | G | E | 3.23 436 C₂₃H₂₈F₃N₃O₂ |
| 89 | 6-(2-Bromo-4- chloro- phenylamino)-N- cyclopentylmethyl-4- isopropyl- nicotinamide | 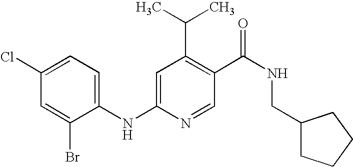 | G | E | 3.97 452 C₂₁H₂₅⁷⁹Br₃₅ClN₃O |
| 90 | 6-(4-Bromo-3- chloro- phenylamino)-4- isopropyl-N- (tetrahydro-pyran-4- ylmethyl)- nicotinamide | 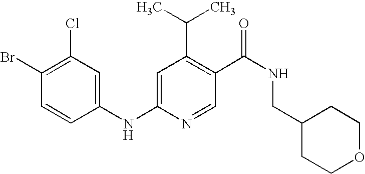 | G | H | 3.48 466 C₂₁H₂₅⁷⁹Br₃₅ClN₃O₂ |
| 91 | 6-(4-Chloro-2-fluoro- phenylamino)-N- cyclopentylmethyl-4- isopropyl- nicotinamide | 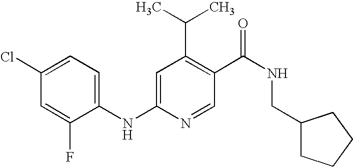 | G | E | 3.7 390 C₂₁H₂₅³⁵ClFN₃O |
| 92 | N- Cyclopentylmethyl- 6-(3-fluoro-4- trifluoromethyl- phenylamino)-4- isopropyl- nicotinamide | 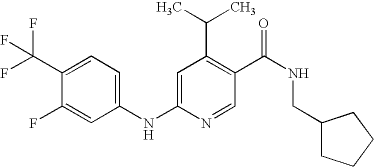 | G | H | 3.8 424 C₂₂H₂₅F₄N₃O |
| 93 | 6-(4-Cyano-2- methyl- phenylamino)-N- cyclopentylmethyl-4- isopropyl- nicotinamide | 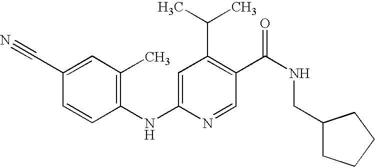 | B | H | 3.43 377 C₂₃H₂₈N₄O |

Preparative Method B As for the preparation of Example 3
Preparative Method G As for the preparation of Example 75
Purification Method A: Purify by trituration as for Example 2.
Purification Method E: Purify by mass-directed autopreparative technique.
Purification Method H: Purify using the Biotage Horizon system detailed at the beginning of the experimental section.

TABLE 9
All compounds in table 9 were prepared as for Example 75 and purified by the technique
given in the table.

| | | | | RT (min), (MH⁺), |
|---|---|---|---|---|
| Ex. | | | Purification | Consistent with the |
| No. | Name | Structure | method | molecular formula |
| 94 | 6-(3-Chloro-2-fluoro- phenylamino)-4- isopropyl-N- (tetrahydro-pyran-4- ylmethyl)-nicotinamide | 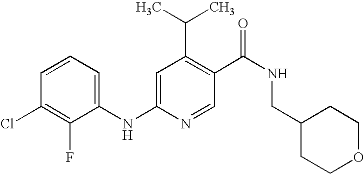 | E | 3.05 406 C₂₁H₂₅³⁵ClFN₃O₂ |
| 95 | 6-(3-Fluoro-4- trifluoromethyl- phenylamino)-4- isopropyl-N- (tetrahydro-pyran-4- ylmethyl)-nicotinamide | 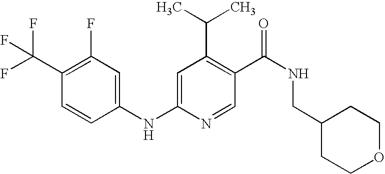 | E | 3.40 440 C₂₂H₂₅F₄N₃O₂ |
| 96 | 6-(4-Cyano-3- trifluoromethyl- phenylamino)-4- isopropyl-N- (tetrahydro-pyran-4- ylmethyl)-nicotinamide | 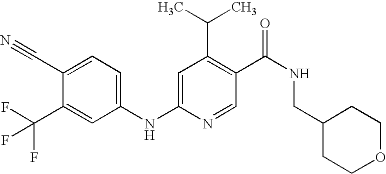 | E | 3.29 447 C₂₃H₂₅F₃N₄O₂ |
| 97 | 6-(4-Cyano-2-fluoro- phenylamino)-4- isopropyl-N- (tetrahydro-pyran-4- ylmethyl)-nicotinamide | 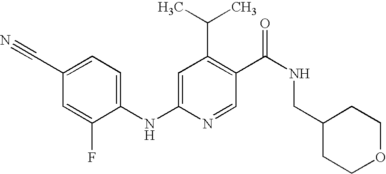 | E | 2.92 397 C₂₂H₂₅FN₄O₂ |
| 98 | 6-(4-fluoro-3-methyl- phenylamino)-4- isopropyl-N- (tetrahydro-pyran-4- ylmethyl)-nicotinamide | 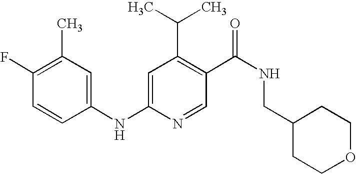 | H | 2.83 386 C₂₂H₂₈FN₃O₂ |
| 99 | 6-(5-Chloro-2-methyl- phenylamino)-4- isopropyl-N- (tetrahydro-pyran-4- ylmethyl)-nicotinamide | 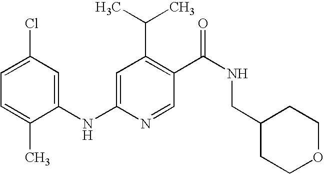 | E | 3.02 402 C₂₂H₂₈³⁵ClN₃O₂ |
| 100 | 6-(3-Fluoro-4-methyl- phenylamino)-4- isopropyl-N- (tetrahydro-pyran-4- ylmethyl)-nicotinamide | 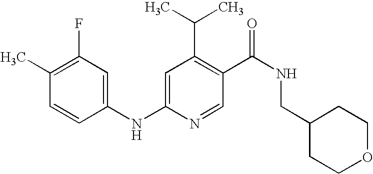 | H | 3.03 386 C₂₂H₂₈FN₃O₂ |
| 101 | 6-(3,4-Dimethyl- phenylamino)-4- isopropyl-N- (tetrahydro-pyran-4- ylmethyl)-nicotinamide | 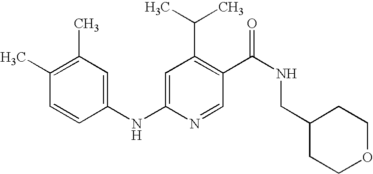 | H | 2.85 382 C₂₃H₃₁N₃O₂ |
| 102 | 6-(3-Bromo-4-methyl- phenylamino)-4- isopropyl-N- (tetrahydro-pyran-4- ylmethyl)-nicotinamide | 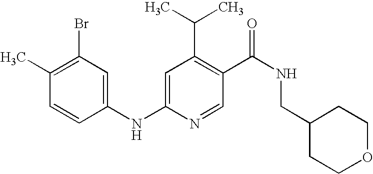 | H | 3.32 446 C₂₂H₂₈⁷⁹BrN₃O₂ |

Purification Method E: Purify by mass-directed autopreparative technique.
Purification Method H: Purify using the Biotage Horizon system detailed at the beginning of the experimental section.

### EXAMPLE 103

### 6-(3-Chloro-phenylamino)-N-(4-hydroxy-tetrahydro-pyran-4-ylmethyl)-4-isopropyl-nicotinamide

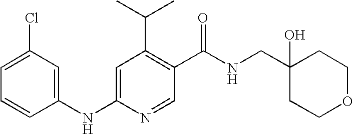

This was prepared by the same method used to prepare Example 50 from Description 16.

LC/MS t=2.89 min, [MH⁺] 404 C₂₁H₂₆³⁵ClN₃O₃

### EXAMPLE 104

### 6-(2,3-Dichloro-phenylamino)-N-(cyclobutyl)-4-trifluoromethyl-nicotinamide

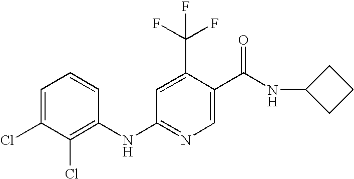

N-methylmorpholine (48 uL, 0.43 mmol), cyclobutylamine (13 mg, 0.18 mmol), 1-hydroxybenzotriazole (30 mg, 0.22 mmol), 1-(3-dimethylamino-propyl)-3-ethylcarbodiimide hydrochloride (32 mg, 0.17 mmol) were added to a solution of 6-(2,3-dichloro-phenylamino)-4-trifluoromethyl nicotinic acid (Description 18) (50 mg, 0.14 mmol) in dimethylformamide (3 mL). After stirring at room temperature for 6 h, dimethylformamide was evaporated under reduced pressure and dichloromethane was added. The solution was washed with an aqueous solution of NaHCO₃ 5% (5 mL), with water (10 mL), then with brine (2×3 mL) and was evaporated under reduced pressure. The crude residue was triturated with diethyl ether, filtered and dried under vacuum to afford the title compound (46 mg, yield=81%).

¹H NMR (300 MHz, DMSO-d₆) δ: 9.27 (s br, 1H); 8.66 (d br, 1H); 8.27 (s, 1H); 7.90 (dd, 1H); 7.42-7.31 (m, 3H); 4.30 (m, 1H); 2,21 (m, 2H); 1.97 (m, 2H); 1.66 (m, 2H).

MS m/z (EI+); TSQ 700; source 180° C.; 70 V; 200 uA: 403 (M⁺), 375, 332.

### EXAMPLE 105

### 6-(2,4-Dichloro-phenylamino)-N-(tetrahydropyran-4-ylmethyl)-4-trifluoromethyl-nicotinamide

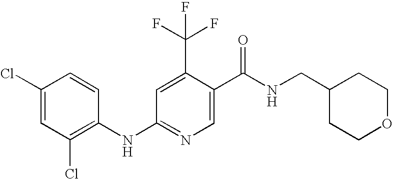

1-Hydroxy-7-azabenzotriazole (33 mg, 0.24 mmol), tetrahydropyran-4-ylmethylamine (17 mg, 0.14 mmol) and PS-carbodiimide (218 mg, 0.28 mmol, loading 1.31 mmol/g, ex Argonaut Technologies) were added to a solution of 6-(2,4-dichloro-phenylamino)-4-trifluoromethyl nicotinic acid (Description 20)(75 mg, 0.21 mmol) in 3 mL of dichloromethane. After orbital shaking at room temperature overnight, the resin was filtered and washed repeatedly with dichloromethane; the filtrate was treated with an aqueous solution of NaHCO₃ 5%. The organic layer was separated through Phase Separator cartridge, dried over sodium sulphate and evaporated in vacuo. The solid residue was triturated with acetonitrile, filtered and dried under vacuum to afford the title compound (44 mg, yield=46%).

¹H NMR (300 MHz, DMSO-d₆) δ: 9.18 (s, 1H); 8.48 (t br, 1H); 8.27 (s, 1H); 7.98 (d, 1H); 7.66 (d, 1H); 7.42 (dd, 1H); 7.37 (s, 1H); 3.84 (dd, 2H); 3.26 (dd, 2H); 3.10 (dd, 1H); 1.74 (m, H); 1.60 (d br, 2H); 1.18 (m, 2H).

MS m/z (EI+); TSQ 700; source 180° C.; 70 V; 200 uA: 447 (M⁺), 412, 333, 314.

### EXAMPLE 106

### 6-(3-Chloro-phenylamino)-N-(1,1-dioxo-tetrahydrothiophen-3-ylmethyl)-4-trifluoromethyl-nicotinamide

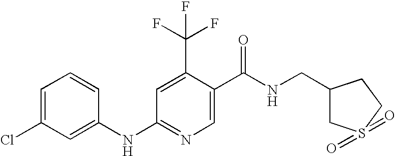

PS-carbodiimide (1.6 g, 2 mmol, loading 1.31 mmol/g, ex Argonaut Technologies) and 1-hydroxy-benzotriazole (0.2 g, 1.5 mmol) were added to a solution of 6-(3-chlorophenylamino)-4-trifluoromethyl nicotinic acid (Description 22) (0.35 g, 1 mmol) in dry dichloromethane (15 mL) and the mixture was stirred at room temperature overnight. The resin was filtered and washed repeatedly with dichloromethane, the solvent was then removed under reduced pressure. The solid residue was dissolved in anhydrous tetrahydrofuran (3.5 mL) and PS-diisopropylethylamine (300 mg, 1.16 mmol, loading 3.88 mmol/g, ex Argonaut Technologies), (1,1-dioxo-tetrahydrothiophen-3-yl)methylamine (0.185 g, 1 mmol) and 1-butyl-3-methylimidazolium hexafluorophosphate (72 uL, 0.35 mmol) were added. The mixture was heated in a sealed tube under microwaves irradiation for 40 min at 140° C. (power=25-30 W), then the resin was filtered and washed with THF (15 mL) and dichloromethane (15 mL) and the filtrate was evaporated under reduced pressure. The residue was dissolved in dichloromethane, washed with an aqueous solution of K₂CO₃ 10%, dried over magnesium sulphate and evaporated under reduced pressure. Purification by flash chromatography on silica gel (initial eluent: DCM, final eluent: DCM/MeOH 98:2) yielded the title compound (210 mg, yield=47%).

¹H NMR (300 MHz, CDCl₃) δ: 8.41 (s, 1H); 8.38 (s, 1H); 7.73 (dd, 1H); 7.37 (d br, 1H); 7.36 (t br, 1H); 7.21 (dd, 1H); 7.04 (s, 1H); 6.98 (d br, 1H); 3.60-3.39 (m, 2H); 3.24-3.12 (m, 2H); 3.02 (ddd, 1H); 2.90-2.70 (m, 2H); 2.38-2.26 (m, 1H); 2.09-1.87 (m, 1H).

MS m/z (EI+); TSQ 700; source 180° C.; 70 V; 200 uA: 447 (M⁺); 299; 236.

TABLE 10
Compounds of Examples 107 to 172 described in Table 10 were prepared as described in
Example 104 (Method A), Example 105 (Method B) and Example 106 (Method C). The
method used is indicated in the third column.

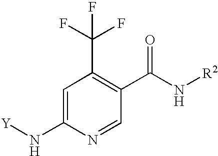

| Ex. | | | | | |
|---|---|---|---|---|---|
| No | Chemical name | Method | Y | R² | ¹H NMR (solvent) ppm and/or MS |
| 107 | N-Cyclohexylmethyl-6- phenylamino-4-trifluoro- methyl-nicotinamide | B | 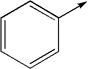 | 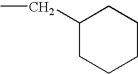 | ESI Pos: AQA; Spray 3.5 kV; Skimmer 30 V; Probe 250° C.: 378 (MH+). |
| 108 | N-Cyclopentylmethyl-6- phenylamino-4-trifluoro- methyl-nicotinamide | B | 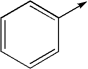 | 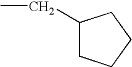 | ESI Pos: AQA; Spray 3.5 kV; Skimmer 30 V; Probe 250° C.: 364 (MH+). |
| 109 | N-Cyclobutylmethyl-6- phenylamino-4-trifluoro- methyl-nicotinamide | B | 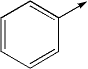 | 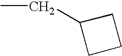 | ESI Pos: AQA; Spray 3.5 kV; Skimmer 30 V; Probe 250° C.: 350 (MH+). |
| 110 | N-Cyclobutyl-6-(3-chloro- phenylamino)-4-trifluoro- methyl-nicotinamide | B | 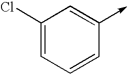 | 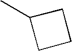 | ¹H NMR (300 MHz, DMSO-d_{8) δ: 9.87 (s, 1H); 8.66 (d br, 1H); 8.40 (s, 1H); 8.01 (dd, 1H); 7.49 (dd, 1H); 7.34 (dd, 1H); 7.16 (s, 1H); 7.02 (dd, 1H); 4.31 (m, 1H); 2.22 (m, 2H); 1.99 (m, 2H); 1.67 (m, 2H). ESI Pos: AQA; Spray 3 kV; Source 20 V; Probe 250° C.: 370 (MH+).} |
| 111 | N-(Tetrahydropyran-4-yl- methyl)-6-(3-chlorophenyl- amino)-4-trifluoromethyl- nicotinamide | B | 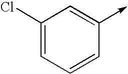 | 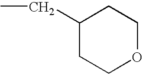 | ESI Pos: AQA; Spray 3.5 kV; Skimmer 30 V; Probe 250° C.: 414 (MH+). |
| 112 | N-Cyclobutylmethyl-6-(3- chlorophenylamino)-4-tri- fluoromethyl-nicotinamide | B | 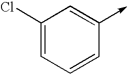 | 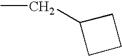 | ESI Pos: AQA; Spray 3.5 kV; Skimmer 30 V; Probe 250° C.: 383 (MH+). |
| 113 | N-Isobutyl-6-(3-chloro- phenylamino)-4-trifluoro- methyl-nicotinainide | B | 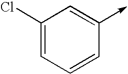 | 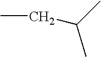 | ESI Pos: AQA; Spray 3.5 kV; Skimmer 30 V; Probe 250° C.: 372 (MH+). |
| 114 | N-Cyclopentylmethyl-6-(3- chlorophenylamino)-4-tri- fluoromethyl-nicotinamide | B | 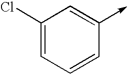 | 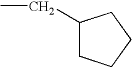 | ESI Pos: AQA; Spray 3.5 kV; Skimmer 30 V; Probe 250° C.: 398 (MH+). |
| 115 | N-Cyclopropylmethyl-6-(3- chlorophenylamino)-4-tri- fluoromethyl-nicotinamide | B | 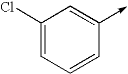 | 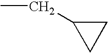 | ESI Pos: AQA; Spray 3.5 kV; Skimmer 30 V; Probe 250° C.: 370 (MH+). |
| 116 | N-Cyclohexylmethyl-6-(3- bromophenylamino)-4-tri- fluoromethyl-nicotinamide | B | 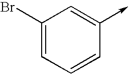 | 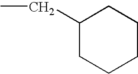 | ESI Pos: AQA; Spray 3.5 kV; Skimmer 30 V; Probe 250° C.: 456 (MH+). |
| 117 | N-Cycloheptylmethyl-6-(3- bromophenylamino)-4-tri- fluoromethyl-nicotinamide | B | 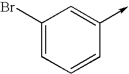 | 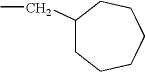 | ESI Pos: AQA; Spray 3.5 kV; Skimmer 30 V; Probe 250° C.: 470 (MH+). |
| 118 | N-(Tetrahydropyran-4-yl- methyl)-6-(3-bromophenyl- amino)-4-trifluoromethyl- nicotinamide | B | 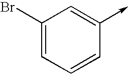 | 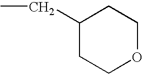 | ESI Pos: AQA; Spray 3.5 kV; Skimmer 30 V; Probe 250° C.: 458 (MH+). |
| 119 | N-Cyclobutyl-6-(3-bromo- phenylamino)-4-trifluoro- methyl-nicotinamide | B | 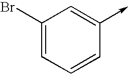 | 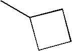 | ESI Pos: AQA; Spray 3.5 kV; Skimmer 30 V; Probe 250° C.: 414 (MH+). |
| 120 | N-Cyclobutylmethyl-6-(3- bromo-phenylamino)-4-tri- fluoromethyl-nicotinamide | B | 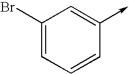 | 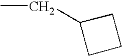 | ESI Pos: AQA; Spray 3.5 kV; Skimmer 30 V; Probe 250° C.: 427 (MH+). |
| 121 | N-Isobutyl-6-(3-bromo- phenylamino)-4-trifluoro- methyl-nicotinamide | B | 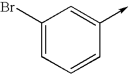 | 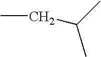 | ESI Pos: AQA; Spray 3.5 kV; Skimmer 30 V; Probe 250° C.: 415 (MH+). |
| 122 | N-Cyclopentylmethyl-6-(3- bromo-phenylamino)-4-tri- fluoromethyl-nicotinamide | B | 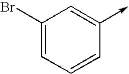 | 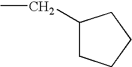 | ESI Pos: AQA; Spray 3.5 kV; Skimmer 30 V; Probe 250° C.: 442 (MH+). |
| 123 | N-Cyclopropylmethyl-6-(3- bromo-phenylamino)-4-tri- fluoromethyl-nicotinamide | B | 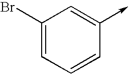 | 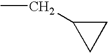 | ESI Pos: AQA; Spray 3.5 kV; Skimmer 30 V; Probe 250° C.: 414 (MH+). |
| 124 | N-Cyclobutylmethyl-6-(2- fluoro-phenylamino)-4-tri- fluoromethyl-nicotinamide | B | 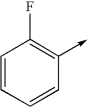 | 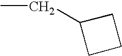 | ESI Pos: AQA; Spray 3.5 kV; Skimmer 30 V; Probe 250° C.: 368 (MH+). |
| 125 | N-Cycloheptylmethyl-6-(3- fluoro-phenylamino)-4-tri- fluoromethyl-nicotinamide | B | 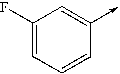 | 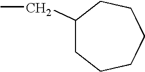 | ESI Pos: AQA; Spray 3.5 kV; Skimmer 30 V; Probe 250° C.: 410 (MH+). |
| 126 | N-(Tetrahydropyran-4-yl- methyl)-6-(3-fluoro-phenyl)- 4-trifluoromethyl- nicotinamide | B | 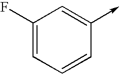 | 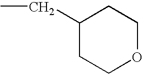 | ESI Pos: AQA; Spray 3.5 kV; Skimmer 30 V; Probe 250° C.: 398 (MH+). |
| 127 | N-Cyclobutyl-6-(3-fluoro- phenylamino)-4-trifluoro- methyl-nicotinamide | B | 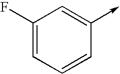 | 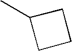 | ESI Pos: AQA; Spray 3.5 kV; Skimmer 30 V; Probe 250° C.: 354 (MH+). |
| 128 | N-Cyclohexylmethyl-6-(3- fluoro-phenylamino)-4-tri- fluoromethyl-nicotinamide | B | 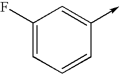 | 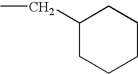 | ESI Pos: AQA; Spray 3.5 kV; Skimmer 30 V; Probe 250° C.: 396 (MH+). |
| 129 | N-Cyclobutylmethyl-6-(3- fluoro-phenylamino)-4-tri- fluoromethyl-nicotinamide | B | 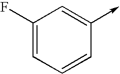 | 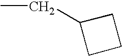 | ESI Pos: AQA; Spray 3.5 kV; Skimmer 30 V; Probe 250° C.: 367 (MH+). |
| 130 | N-Cyclopentylmethyl-6-(3- fluoro-phenylamino)-4-tri- fluoromethyl-nicotinamide | B | 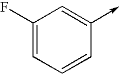 | 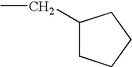 | ESI Pos: AQA; Spray 3.5 kV; Skimmer 30 V; Probe 250° C.: 382 (MH+). |
| 131 | N-Isobutyl-6-(3-fluoro- phenylamino)-4-trifluoro- methyl-nicotinamide | B | 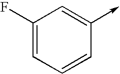 | 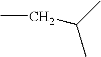 | ESI Pos: AQA; Spray 3.5 kV; Skimmer 30 V; Probe 250° C.: 356 (MH+). |
| 132 | N-Cyclopropylmethyl-6-(3- fluoro-phenylamino)-4-tri- fluoromethyl-nicotinamide | B | 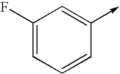 | 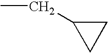 | ESI Pos: AQA; Spray 3.5 kV; Skimmer 30 V; Probe 250° C.: 354 (MH+). |
| 133 | N-(1,1-Dioxo-tetrahydro- thiophen-3-ylmethyl)-6-(3- fluoro-phenylamino)-4- trifluoromethyl-nicotinamide | B | 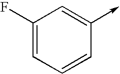 | 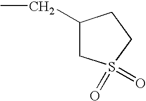 | ESI Pos: AQA; Spray 3.5 kV; Skimmer 30 V; Probe 250° C.: 432 (MH+). |
| 134 | N-Cyclobutylmethyl-6-(4- fluoro-phenylamino)-4- trifluoromethyl-nicotinamide | B | 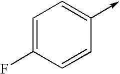 | 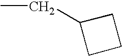 | ESI Pos: AQA; Spray 3.5 kV; Skimmer 30 V; Probe 250° C.: 368 (MH+). |
| 135 | N-(Tetrahydropyran-4-yl- methyl)-6-(2,3-dichloro- phenylamino)-4-trifluoro- methyl-nicotinanhide | B | 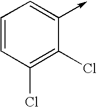 | 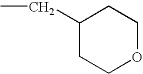 | ESI Pos: AQA; Spray 3.5 kV; Skimmer 30 V; Probe 250° C.: 448 (MH+). |
| 136 | N-Cyclohexylmethyl-(2,3- dichloro-phenylamino)-4-tri- fluoromethyl-nicotinamide | B | 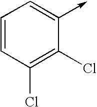 | 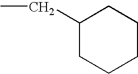 | ESI Pos: AQA; Spray 3.5 kV; Skimmer 30 V; Probe 250° C.: 446 (MH+). |
| 137 | N-Cycloheptylmethyl-6-(2,3- dichloro-phenylamino)-4-tri- fluoromethyl-nicotinamide | B | 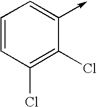 | 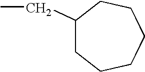 | ESI Pos: AQA; Spray 3.5 kV; Skimmer 30 V; Probe 250° C.: 460 (MH+). |
| 138 | N-Cyclohexylmethyl-6-(2,4- dichloro-phenylamino)-4-tri- fluoromethyl-nicotinamide | B | 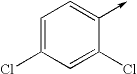 | 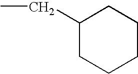 | ESI Pos: AQA; Spray 3.5 kV; Skimmer 30 V; Probe 250° C.: 447 (MH+). |
| 139 | N-Cycloheptylmethyl-6-(2,4- dichloro-phenylamino)-4-tri- fluoromethyl-nicotinamide | B | 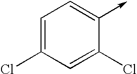 | 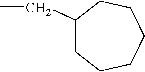 | ESI Pos: AQA; Spray 3.5 kV; Skimmer 30 V; Probe 250° C.: 460 (MH+). |
| 140 | N-Cyclobutyl-6-(2,4-dichloro- phenylamino)-4-trifluoro- methyl-nicotinamide | B | 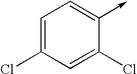 | 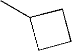 | ESI Pos: AQA; Spray 3.5 kV; Skimmer 30 V; Probe 250° C.: 404 (MH+). |
| 141 | N-Cyclopentylmethyl-6-(2,4- dichloro-phenylamino)-4-tri- fluoromethyl-nicotinamide | B | 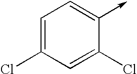 | 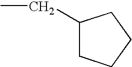 | ESI Pos: AQA; Spray 3.5 kV; Skimmer 30 V; Probe 250° C.: 432 (MH+). |
| 142 | N-Cyclobutylmethyl-6-(2,4- dichloro-phenylamino)-4-tri- fluoromethyl-nicotinamide | B | 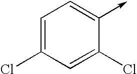 | 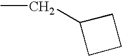 | ESI Pos: AQA; Spray 3.5 kV; Skimmer 30 V; Probe 250° C.: 418 (MH+). |
| 143 | N-Isobutyl-6-(2,4-dichloro- phenylamino)-4-trifluoro- methyl-nicotinamide | B | 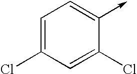 | 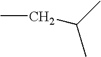 | ESI Pos: AQA; Spray 3.5 kV; Skimmer 30 V; Probe 250° C.: 406 (MH+). |
| 144 | N-Cyclopropylmethyl-6-(2,4- dichloro-phenylamino)-4-tri- fluoromethyl-nicotinamide | B | 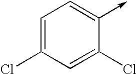 | 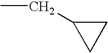 | ESI Pos: AQA; Spray 3.5 kV; Skimmer 30 V; Probe 250° C.: 404 (MH+). |
| 145 | N-(1,1-Dioxo-tetrahydro-thio- phen-3-ylmethyl)-6-(2,4-di- chloro-phenylamino)-4- C | 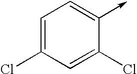 | 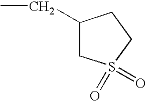 | ¹H NMR (300 MHz, CDCl₃) δ: 8.38 (s, 1H); 8.08 (d, 1H); 7.47 (s, 1H); 7.41 (t br, 1H); 7.40 (d, 1H); 7.23 (dd, 1H); 7.04 (s, 1H); 3.60-3.39 (m, | |
| # 2H); 3.24-3.12 (m, 2H); 3.01 (ddd, 1H); 2.90-2.72 (m, 2H); 2.38-2.26 (m, 1H); 2.09-1.87 (m, 1H). EI+; TSQ 700; source 180° C.; 70 V; 200 uA: 481 (M⁺); 446; 333; 270. | | | | | |
| 146 | N-Cyclohexylmethyl-6-(3,5- dichloro-phenytamino)-4-tri- fluoromethyl-nicotinamide | B | 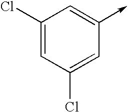 | 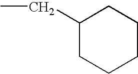 | ESI Pos: AQA; Spray 3.5 kV; Skimmer 30 V; Probe 250° C.: 446 (MH+). |
| 147 | N-(Tetrahydropyran-4-yl- methyl)-6-(3,5-dichloro- phenylamino)-4-trifluoro- methyl-nicotinamide | B | 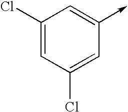 | 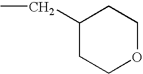 | ESI Pos: AQA; Spray 3.5 kV; Skimmer 30 V; Probe 250° C. 448 (MH+). |
| 148 | N-Cyclobutyl-6-(3,5-dichloro- phenylamino)-4-trifluoro- methyl-nicotinamide | B | 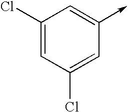 | 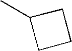 | ESI Pos: AQA; Spray 3.5 kV; Skimmer 30 V; Probe 250° C.: 404 (MH+). |
| 149 | N-Cyclopentylmethyl-6-(3,5- dichloro-phenylamino)-4-tri- fluoromethyl-nicotinamide | B | 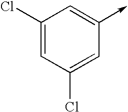 | 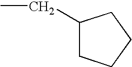 | ESI Pos: AQA; Spray 3.5 kV; Skimmer 30 V; Probe 250° C.: 432 (MH+). |
| 150 | N-Cyclobutylmethyl-6-(3,5- dichloro-phenylamino)-4-tri- fluoromethyl-nicotinamide | B | 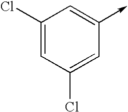 | 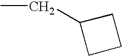 | ESI Pos: AQA; Spray 3.5 kV; Skimmer 30 V; Probe 250° C.: 418 (MH+). |
| 151 | N-Isobutyl-6-(3,5-dichloro- phenylamino)-4-trifluoro- methyl-nicotinamide | B | 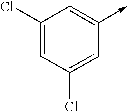 | 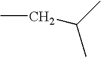 | ESI Pos: AQA; Spray 3.5 kV; Skimmer 30 V; Probe 250° C.: 406 (MH+). |
| 152 | N-Cyclopropylmethyl-6-(3,5- dichloro-phenylamino)-4-tri- fluoromethyl-nicotinamide | B | 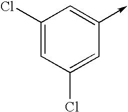 | 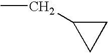 | ESI Pos: AQA; Spray 3.5 kV; Skimmer 30 V; Probe 250° C.: 404 (MH+). |
| 153 | N-Isobutyl-6-(3,4-dichloro- phenylamino)-4-trifluoro- methyl-nicotinamide | B | 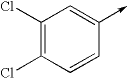 | 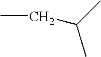 | ¹H NMR (300 MHz, DMSO-d₆) δ: 9.98 (s br, 1H); 8.47 (t br, 1H); 8.41 (s, 1H); 8.20 (s, 1H); 7.55 (s, 2H); 7.17 (s, 1H); 3.05 (dd, 2H); 1.80 (m, 1H); 0.90 (d, 6H). ESI Pos: AQA; Spray 3 kV; Source 20 V; Probe 250° C.: 406 (MH+). |
| 154 | N-Cyclobutyl-6-(3,4-dichloro- phenylamino)-4-trifluoro- methyl-nicotinamide | B | 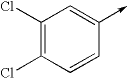 | 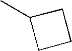 | ESI Pos: AQA; Spray 3.5 kV; Skimmer 30 V; Probe 250° C.: 404 (MH+). |
| 155 | N-(Tetrahydropyran-4-yl- methyl)-6-(3,4-dichloro- phenylamino)-4-trifluoro- methyl-nicotinamide | B | 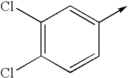 | 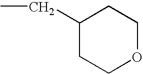 | ESI Pos: AQA; Spray 3.5 kV; Skimmer 30 V; Probe 250° C.: 448 (MH+). |
| 156 | N-Cyclopentylmethyl-6-(3,4- dichloro-phenylamino)-4-tri- fluoromethyl-nicotinamide | B | 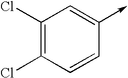 | 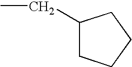 | ESI Pos: AQA; Spray 3.5 kV; Skimmer 30 V; Probe 250° C.: 432 (MH+). |
| 157 | N-Cyclobutylmethyl-6-(3,4- dichloro-phenylamino)-4-tri- fluoromethyl-nicotinamide | B | 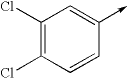 | 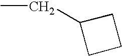 | ESI Pos: AQA; Spray 3.5 kV; Skimmer 30 V; Probe 250° C.: 418 (MH+). |
| 158 | N-Cyclopropylmethyl-6-(3,4- dichloro-phenylamino)-4-tri- fluoromethyl-nicotinamide | B | 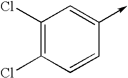 | 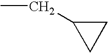 | ESI Pos: AQA; Spray 3.5 kV; Skimmer 30 V; Probe 250° C.: 404 (MH+). |
| 159 | N-Cyclohexylmethyl-6-(3,4- dichloro-phenylamino)-4-tri- fluoromethyl-nicotinamide | B | 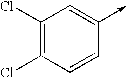 | 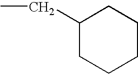 | ESI Pos: AQA; Spray 3.5 kV; Skimmer 30 V; Probe 250° C.: 446 (MH+). |
| 160 | N-Cyclobutylmethyl-6-(2- fluoro-4-chloro-phenyl- amino)-4-trifluoromethyl- nicotinamide | A | 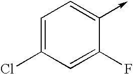 | 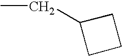 | ¹H NMR (300 MHz, DMSO-d₆) δ: 9.42 (s, 1H); 8.42 (t br, 1H); 8.28 (s, 1H); 8.17 (dd, 1H); 7.48 (dd, 1H); 7.35 (s, 1H); 7.27 (d br, 1H); 3.23 (dd, |
| # 211); 2.48 (m, 1H); 2.04-1.91 (m, 2H); 1.89-1.64 (m, 4H). EI+; TSQ 700; source 180° C.; 70 V; 200 uA: 401 (M⁺) 366, 333, 317. | | | | | |
| 161 | N-Cyclopentylmethyl-6-(2- fluoro-4-chloro-phenyl- amino)-4-trifluoromethyl- nicotinamide | A | 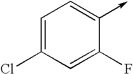 | 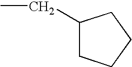 | ¹H NMR (300 MHz, DMSO-d₆) δ: 9.42 (s, 1H); 8.47 (t br, 1H); 8.29 (s, 1H); 8.17 (dd, 1H); 7.48 (dd, 1H); 7.35 (s, 1H); 7.27 (d br, 1H); 3.14 (dd, 2H); 2.08 (m, 1H); 1.75-1.42 (m, 6H); 1.29-1.15 |
| # (m, 2H). EI+; TSQ 700; source 180° C.; 70 V; 200 uA: 415 (M⁺) 346, 333, 317. | | | | | |
| 162 | N-(Tetrahydropyran-4-yl- methyl)-6-(2-fluoro-4-chloro- phenylamino)-4-trifluoro- methyl-nicotinamide | A | 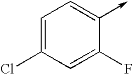 | 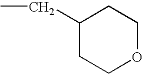 | ¹H NMR (300 MHz, DMSO-d₆) 5: 9.44 (s, 1H); 8.50 (t br, 1H); 8.32 (s, 1H); 8.17 (dd, 1H); 7.48 (s, 1H); 7.29 (d br, 1H); 3.84 (dd, 2H); 3.26 (dd, 2H); 3.11 (dd, 2H); 1.74 (m, 1H); 1.60 (m, 2H); |
| # 1.19 (m, 2H). EI+; TSQ 700; source 180° C.; 70 V; 200 uA: 431 (M⁺); 346; 333; 317. | | | | | |
| 163 | N-Cyclobutylmethyl-6-(2- Chloro-4-fluoro-phenyl- amino)-4-trifluoromethyl- nicotinamide | A | 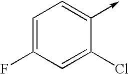 | 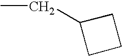 | ¹H NMR (300 MHz, DMSO-d₆) δ: 9.13 (s, 1H); 8.39 (t br, 1H); 8.19 (s, 1H); 7.80 (dd, 1H); 7.51 (dd, 1H); 7.24 (dt, 1H); 7.20 (s, 1H); 3.22 (dd, |
| # 2H); 2.55-2.42 (m, 1H); 2.04-1.63 (m, 6H). EI+; TSQ 700; source 180° C.; 70 V; 200 uA: 401 (M⁺); 366; 317; 298; 254. | | | | | |
| 164 | N-Cyclopentylmethyl-6-(2- Chloro-4-fluoro-phenyl- amino)-4-trifluoromethyl- nicotinamide | A | 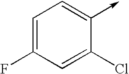 | 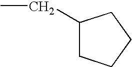 | ¹H NMR (300 MHz, DMSO-d₆) δ: 9.13 (s, 1H); 8.42 (t br, 1H); 8.20 (s, 1H); 7.81 (dd, 1H); 7.52 (dd, 1H); 7.24 (dt, 1H); 7.20 (s, 1H); 3.13 (dd, 2H); 2.07 (m, 1H); 1.75-1.42 (m, 6H); 1.30-1.15 |
| # (m, 2H). EI+; TSQ 700; source 180° C.; 70 V; 200 uA: 415 (M⁺); 380; 346: 317; 298; 254. | | | | | |
| 165 | N-(Tetrahydropyran-4-yl- methyl)-6-(2-chloro-4-fluoro- phenylamino)-4-trifluoro- methyl-nicotinamide | A | 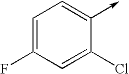 | 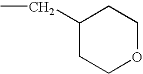 | ¹H NMR (300 MHz, DMSO-d₆) δ: 9.14 (s, 1H); 8.45 (t br, 1H); 8.23 (s, 1H); 7.81 (dd, 1H); 7.51 (dd, 1H); 7.24 (dt, 1H); 7.20 (s, 1H); 3.84 (dd, 2H); 3.25 (dd, 2H); 3.10 (dd, 2H); 1.73 (m, 1H); |
| # 1.59 (m, 2H); 1.18 (m, 2H). EI+; TSQ 700; source 180° C.; 70 V; 200 uA: 431.1 (M⁺), 346, 333, 317. | | | | | |
| 166 | N-Cyclobutylmethyl-6-(2,4- difluoro-phenylamino)-4-tri- fluoromethyl-nicotinamide | A | 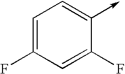 | 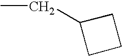 | ¹H NMR (300 MHz, DMSO-d₆) δ: 9.28 (s, 1H); 8.39 (t br, 1H); 8.23 (s, 1H); 7.95 (m, 1H); 7.31 (ddd, 1H); 7.21 (s, 1H); 7.08 (t br, 1H); 3.24 (dd, |
| # 2H); 2.55-2.42 (m, 1H); 2.04-1.63 (m, 6H). EI+; TSQ 700; source 180° C.; 70 V; 200 uA: 385 (M⁺); 366; 317; 301. | | | | | |
| 167 | N-Cyclopentylmethyl-6-(2,4- difluoro-phenylamino)-4-tri- fluoromethyl-nicotinamide | A | 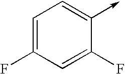 | 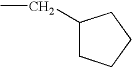 | ¹H NMR (300 MHz, DMSO-d₆) δ: 9.29 (s, 1H); 8.45 (t br, 1H); 8.24 (s, 1H); 7.96 (dt, 1H); 7.32 (ddd, 1H); 7.22 (s, 1H); 7.09 (t br, 1H); 3.13 (dd, 2H); 2.08 (m, 1H); 1.75-1.42 (m, 6H); 1.30-1.16 |
| # (m, 2H). EI+; TSQ 700; source 180° C.; 70 V; 200 uA: 399 (M⁺); 380; 330; 317; 301; 298. | | | | | |
| 168 | N-(Tetrahydropyran-4-yl- methyl)-6-(2-methoxy-5- chloro-phenylainino)-4-tri- fluoromethyl-nicotinamide | B | 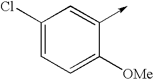 | 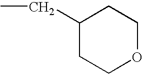 | ESI Pos: AQA; Spray 3.5 kV; Skimmer 30 V; Probe 250° C.: 445 (MH+). |
| 169 | N-Cyclobutylmethyl-6-(2- methoxy-5-chloro-phenyl- amino)-4-trifluoromethyl- nicotinamide | B | 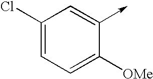 | 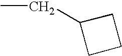 | ESI Pos: AQA; Spray 3.5 kV; Skimmer 30 V; Probe 250° C.: 415 (MH+). |
| 170 | N-(Tetrahydropyran-4-yl- methyl)-6-(2-hydroxy-5- chloro-phenylamino)-4- trifluoromethyl-nicotinamide | A | 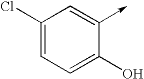 | 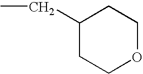 | ESI Pos: AQA; Spray 3.5 kV; Skimmer 30 V; Probe 250° C.: 430 (MH+). |
| 171 | N-Cyclohexylmethyl-6-(2- methyl-4-chloro-phenyl- amino)-4-trifluoromethyl- nicotinamide | A | 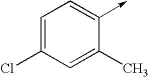 | 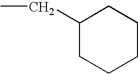 | ¹H NMR (300 MHz, DMSO-d₈) δ: 8.89 (s br, 1H); 8.36 (t br, 1H); 8.21 (s, 1H); 7.62 (d, 1H); 7.33 (d, 1H); 7.24 (dd, 1H); 7.12 (s, 1H); 3.04 (dd, 2H); 2.23 (s, 3H); 1.76-1.39 (m, 6H); 1.29- |
| # 1.05 (m, 3H); 0.99-0.83 (m, 2H). EI+; TSQ 700; source 180° C.; 70 V; 200 uA: 425 (M⁺); 410; 342; 329; 313. | | | | | |
| 172 | N-(Tetrahydropyran-4-yl- methyl)-6-(2-methyl-4-chloro- phenylamino)-4-trifluoro- A | 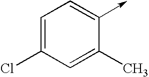 | 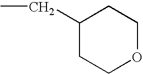 | ¹H NMR (300 MHz, DMSO-d₈) δ: 8.91 (s br, 1H); 8.42 (t br, 1H); 8.23 (s, 1H); 7.63 (d, 1H); 7.32 (d, 1H); 7.24 (dd, 1H); 7.12 (s, 1H); 3.84 (m, 2H); 3.26 (m, 2H); 3.09 (dd, 2H); 2.23 (s, 3H); 1.82-1.65 (m, 1H); 1.58 (d br, 2H); 1.18 | |
| # (dq, 2H). EI+; TSQ 700; source 180° C.; 70 V; 200 uA: 427 (M⁺); 412; 313. | | | | | |

TABLE 11
Compounds of Examples 173 to 177 described in Table 11 were prepared as described in Example 104 (Method A),
Example 105 (Method B) and Example 106 (Method C). The method used is indicated in the third column.

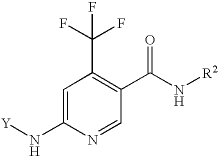

| Ex. | | | | | |
|---|---|---|---|---|---|
| No. | Chemical name | Method | Y | R² | ¹H NMR (solvent) ppm and/or MS |
| 173 | N-(Tetrahydropyran-4-ylmethyl)-6- phenylamino-4-trifluoromethyl- nicotinamide | B | 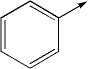 | 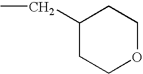 | ESI Pos: AQA; Spray 3.5 kV; Skimmer 30 V; Probe 250° C.: 380 (MH+). |
| 174 | N-Cyclopropylmethyl-6- phenylamino-4-trifluoromethyl- nicotinamide | B | 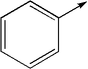 | 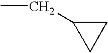 | ESI Pos: AQA; Spray 3.5 kV; Skimmer 30 V; Probe 250° C.: 336 (MH+). |
| 175 | N-Cycloheptylmethyl-6-(3,5- dichloro-phenyiamino)-4- trifluoromethyl-nicotinamide | B | 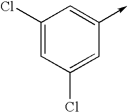 | 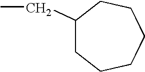 | ESI Pos: AQA; Spray 3.5 kV; Skimmer 30 V; Probe 250° C.: 460 (MH+). |
| 176 | N-(Tetrahydropyran-4-ylmethyl)-6- (2-methyl-5-chloro-phenylamino)-4- trifluoromethyl-nicotina A | 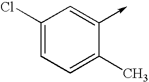 | 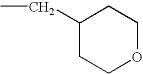 | ¹H NMR (300 MHz, DMSO-d₆) δ: 8.89 (s br, 1H); 8.45 (t br, 1H); 8.30 (s, 1H); 7.86 (d, 1H); 7.26 (s, 1H); 7.25 (d, 1H); 7.07 (dd, 1H); 3.84 (m, 2H); 3.27 (m, 2H); 3.10 (dd, 2H); 2.23 (s, 3H); 1.83-1.68 (m, 1H); 1.60 | |
| # (m, 2H); 1.27-1.10 (m, 2H). EI+; TSQ 700; source 180° C.; 70 V; 200 uA: 427 (M⁺); 412; 313. | | | | | |
| 177 | N-Cyclobutylmethyl-6-(2-hydroxy-5- chloro-phenylamino)-4- trifluoromethyl-nicotmamide | A | 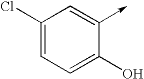 | 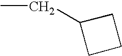 | ESI Pos: AQA; Spray 3.5 kV; Skimmer 30 V; Probe 250° C.: 400 (MH+). |

### EXAMPLE 196

### N-5-Oxo-pyrrolidin-3-ylmethyl)-6-(2,4-dichloro-phenylamino)-4-trifluoromethyl-nicotinamide

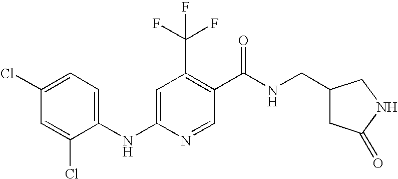

PS-carbodiimide (0.305 g, 0.4 mmol, loading 1.31 mmol/g, ex Argonaut Technologies) and 1-hydroxy-7-azabenzotriazole (0.046 g, 0.34 mmol) were added to a solution of 6-(2,4-dichlorophenylamino)-4-(trifluoromethyl)-nicotinic acid (Description 20) (0.08 g, 0.22 mmol) in dry dichloromethane (5 mL) and the mixture was stirred at room temperature overnight. The resin was filtered and washed repeatedly with dichloromethane, the solvent was then removed in vacuo. The solid residue was dissolved in anhydrous N-methylpyrrolidone (1 mL) and 4-aminomethyl-pyrrolidin-2-one (23 mg, 0.20 mmol) was added. The solution was heated in a sealed tube under microwaves irradiation for 30 min at 140° C. (power=50 M). The reaction mixture was diluted with dichloromethane, washed with an aqueous solution of K₂CO₃ 10%, dried over magnesium sulphate and evaporated under reduced pressure. Chromatographic purification through preparative HPLC on a Symmetry C₁₈ column, by gradient elution with a solvent system water/TFA 99.9:0.1 respectively (A) and CH₃CN/TFA 99.9:0.1 respectively (B) with the following gradient: 5% B (3 min); 5% B→95% B (11 min); 95% B (1 min); 95% B→5% B (2 min) afforded the title compound as its trifluoroacetate salt that was suspended in dichloromethane and treated with NaOH 0.5 N. The organic layer was dried over Na₂SO₄ and evaporated under reduced pressure to give the title compound (42 mg, yield=47%).

ESI Pos: AQA; Spray 3.5 kV; Skimmer 30V; Probe 250° C.: 447 (MH+).

TABLE 12
Compounds of Examples 178 to 201 described in Table 12 were prepared as described in
Example 104 (Method A), Example 105 (Method B) and Example 196 (Method D). The
method used is indicated in the third column.

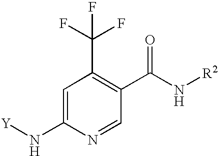

| Ex | | | | | |
|---|---|---|---|---|---|
| No | Chemical name | Method | Y | R² | 1h NMR(Solvent) ppm and/or MS |
| 178 | N-Cycloheptylmethyl-6- phenylamino-4-tri- fluoromethyl-nicotin- amide | B | 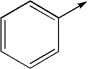 | 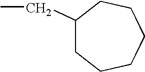 | ESI Pos: AQA; Spray 3.5 kV; Skimmer 30 V; Probe 250° C.: 392 (MH+). |
| 179 | N-Cyclobutyl-6-phenyl- amino-4-trifluoromethyl- nicotinamide | B | 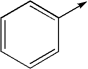 | 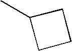 | ESI Pos: AQA; Spray 3.5 kV; Skimmer 30 V; Probe 250° C.: 336 (MH+). |
| 180 | N-Isobutyl-6-phenyl- amino-4-trifluoromethyl- nicotinamide | B | 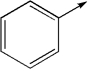 | 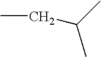 | ESI Pos: AQA; Spray 3.5 kV; Skimmer 30 V; Probe 250° C.: 338 (MH+). |
| 181 | N-(3-Dimethylamino- 2,2-dimethyl-propyl)-6- (3-chloro-phenylamino)- 4-trifluoromethyl- nicotinamide | B | 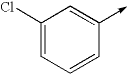 | 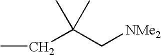 | ESI Pos: AQA; Spray 3.5 kV; Skimmer 30 V; Probe 250° C.: 429 (MH+). |
| 182 | N-(3-Hydroxy-2,2- dimethyl-propyl)-6-(3- chloro-phenylamlno)-4- trifluoromethyl- nicotinamide | B | 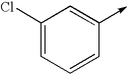 | 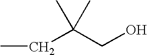 | ESI Pos: AQA; Spray 3.5 kV; Skimmer 30 V; Probe 250° C.: 402 (MH+). |
| 183 | N-(2-Methoxy-2-methyl- propyl)-6-(3-chloro- phenylamino)-4-trifluoro- methyl-nicotinamide | D | 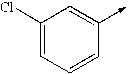 | 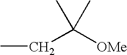 | ESI Pos: AQA; Spray 3.5 kV; Skimmer 30 V; Probe 250° C.: 402 (MH+). |
| 184 | N-([1,4]dioxan-2-yl- methyl)-6-(3-chloro- phenylamino)-4-trifluoro- methyl-nicotinamide | B | 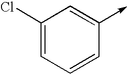 | 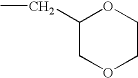 | ESI Pos: AQA; Spray 3.5 kV; Skimmer 30 V; Probe 250° C.: 416 (MH+). |
| 185 | N-(Piperidin-2-yl- methyl)-6-(3-trifluoro- methyl-nicotinamide | B | 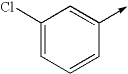 | 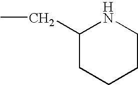 | ESI Pos: AQA; Spray 3.5 kV; Skimmer 30 V; Probe 250° C.: 427 (MH+). |
| 186 | N-(1-Benzyl-5-oxo- pyrrolidin-3-ylmethyl)- 6-(3-chloro-phenyl- amino)-4-trifluoro- methyl-nicotinamide | D | 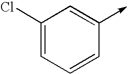 | 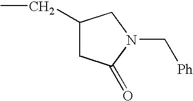 | ESI Pos: AQA; Spray 3.5 kV; Skimmer 30 V; Probe 250° C.: 503 (MH+). |
| 187 | N-(5-Oxo-pyrrolidin-3- ylmethyl)-6-(3-chloro- phenylamino)-4-trifluoro- methyl-nicotinamide | D | 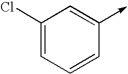 | 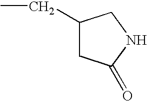 | ESI Pos: AQA; Spray 3.5 kV; Skimmer 30 V; Probe 250° C.: 413 (MH+). |
| 188 | N-Methylcarbamoyl- methyl-6-(3-chloro- phenylamino)-4-trifluoro- methyl-nicotinamide | D | 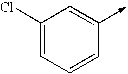 | 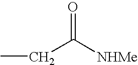 | ESI Pos: AQA; Spray 3.5 kV; Skimmer 30 V; Probe 250° C.: 387 (MH+). |
| 189 | N-(1-Ethyl-pyrrolidin-2- ylmethyl)-6-(3-chloro- phenylamino)-4-trifluoro- methyl-nicotinamide | D | 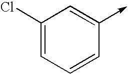 | 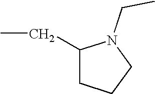 | ESI Pos: AQA; Spray 3.5 kV; Skimmer 30 V; Probe 250° C.: 427 (MH+). |
| 190 | N-(2,2,6,6-Tetramethyl- piperidin-4-ylmethyl)-6- (3-chloro-phenylamino)- 4-tiifluoromethyl- nicotinamide | D | 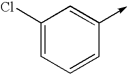 | 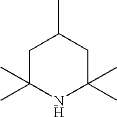 | ESI Pos: AQA; Spray 3.5 kV; Skimmer 30 V; Probe 250° C.: 455 (MH+). |
| 191 | N-(2,2-Dimethyl-[1,3]dioxolan-4-ylmethyl)-6- (3-chloro-phenylamino)- 4-trifluoromethyl- nicotinamide | B | 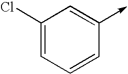 | 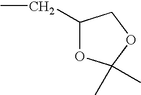 | ESI Pos: AQA; Spray 3.5 kV; Skimmer 30 V Probe 250° C.: 430 (MH+). |
| 192 | N-(Tetrahydropyran-4- ylmethyl)-6-(2-fluoro- phenylamino)-4-trifluoro- methyl-nicotinamide | B | 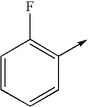 | 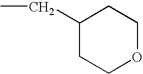 | ESI Pos: AQA; Spray 3.5 kV; Skimmer 30 V; Probe 250° C.: 398 (MH+). |
| 193 | N-(Tetrahydropyran-4- ylmethyl)-6-(4-fluoro- phenylamino)-4-trifluoro- methyl-nicotinamide | B | 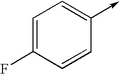 | 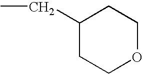 | ESI Pos: AQA; Spray 3.5 kV; Skimmer 30 V; Probe 250° C.: 398 (MH+). |
| 194 | N-(3-Dimethylamino- 2,2-dimethyl-propyl)-6- (2,4-dichloro-phenyl- amino)-4-trifluoro- methyl-nicotinamide | B | 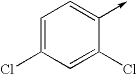 | 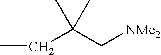 | ESI Pos: AQA; Spray 3.5 kV; Skimmer 30 V; Probe 250° C.: 463 (MH+). |
| 195 | N-([1,4]dioxan-2-yl- methyl)-6-(2,4-dichloro- phenylamino)-4-trifluoro- methyl-nicotinamide | B | 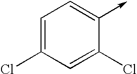 | 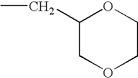 | ESI Pos: AQA; Spray 3.5 kV; Skimmer 30 V; Probe 250° C.: 450 (MH+). |
| 196-see above for full write | N-(5-Oxo-pyrrolidin-3- ylmethyl)-6-(2,4- dichloro-phenylamino)- 4-trifluoromethyl- nicotinamide | D | 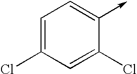 | 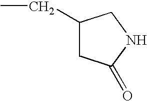 | ESI Pos: AQA; Spray 3.5 kV; Skimmer 30 V; Probe 250° C.: 447 (MH+). |
| 197 | N-Methylcarbamoyl- methyl-6-(2,4-dichloro- phenylamino)-4-trifluoro- methyl-nicotinamide | D | 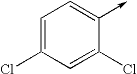 | 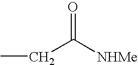 | ESI Pos: AQA; Spray 3.5 kV; Skimmer 30 V; Probe 250° C.: 421 (MH+). |
| 198 | N-(2,2-Dimethyl-[1,3]dioxolan-4-ylmethyl)-6- (2,4-dichloro-phenyl- amino)-4-trifluormethyl- nicotinamide | B | 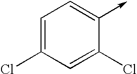 | 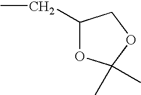 | ESI Pos: AQA; Spray 3.5 kV; Skimmer 30 V; Probe 250° C.: 464 (MH+). |
| 199 | N-Cycloheptylmethyl-6- (3,4-dichloro-phenyl- amino)-4-trifluoro- methyl-nicotinamide | B | 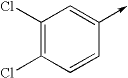 | 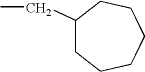 | ESI Pos: AQA; Spray 3.5 kV; Skimmer 30 V; Probe 250° C.: 460 (MH+). |
| 200 | N-(Tetrahydropyran-4-yl- methyl)-6-(2,4-difluoro- phenylamino)-4-trifluoro- methyl-nicotinamide | A | 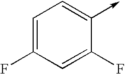 | 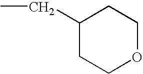 | EI+; TSQ 700; source 180° C.; 70 V; 200 uA: 415 (M⁺). |
| 201 | N-Cyclohexylmethyl-6- (2-methyl-5- chlorophenyl-amino)-4- trifluoromethyl- nicotinamide | A | 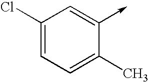 | 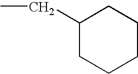 | ESI Pos: AQA; Spray 3.5 kV; Skimmer 30 V; Probe 250° C.: 431 (MH+). |

### EXAMPLE 202

### N-(2,3-Dihydroxy-propyl)-6-(3-chloro-phenylamino)-4-trifluoromethyl-nicotinamide

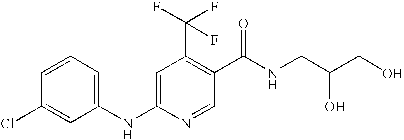

N-(2,2-dimethyl-[1,3]dioxolan-4-ylmethyl)-6-(3-chloro-phenylamino)-4-trifluoromethyl-nicotinamide (Example 191) (30 mg, 0.07 mmol), was dissolved in tetrahydrofuran (4 mL) and stirred overnight at ambient temperature in the presence of Et₂O/HCl (3 mL). Evaporation of the solvent in vacuo afforded the title compound as a white solid (27 mg, yield=99%).

¹H NMR (300 MHz, DMSO-d₆) δ: 9.90 (s, 1H); 8.45 (s, 1H); 8.41 (t br, 1H); 8.02 (dd, 1H); 7.50 (ddd, 1H); 7.34 (dd, 1H); 7.17 (s, 1H); 7.03 (ddd, 1H); 3.65-3.30 (m, 7H); 3.14 (ddd, 1H).

MS m/z (ESI+): AQA; Spray 3.5 kV; Skimmer 30V; Probe 250° C.: 390 (MH+).

### EXAMPLE 203

### N-(2,3-Dihydroxy-propyl)-6-(2,4-dichloro-phenylamino)-4-trifluoromethyl-nicotinamide

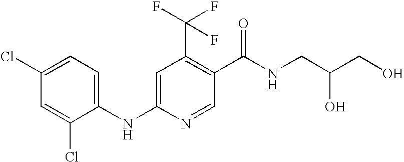

The title compound was prepared in a similar manner to that described in the Example 202, starting from N-(2,2-dimethyl-[1,3]dioxolan-4-ylmethyl)-6-(2,4-dichloro-phenylamino)-4-trifluoromethyl-nicotinamide (Example 199) (40 mg, 0.09 mmol) and the title compound was obtained as a white solid (35 mg, yield=96%).

¹H NMR (300 MHz, CDCl₃) δ: 8.36 (s, 1H); 8.02 (d, 1H); 7.66 (s br, 1H); 7.35 (d, 1H); 7.18 (dd, 1H); 7.11 (t br, 1H); 7.05 (s, 1H); 3.89 (s br, 1H); 3.77 (s br, 1H); 3.59-3.47 (m, 3H); 3.42 (ddd, 1H).

MS m/z (ESI+): AQA; Spray 3.5 kV; Skimmer 30V; Probe 250° C.: 424 (MH+).

### EXAMPLE 204

### 6-(3-Chloro-phenylamino)-N-(tetrahydro-pyran-4-ylmethyl)-2-trifluoromethyl-nicotinamide

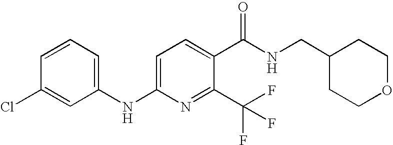

N-methyl morpholine (0.14 mL, 1.27 mmol, 2.5 eq), 1-hydroxy-benzotriazole (110 mg, 0.76 mmol, 1.5 eq), N-(3-dimethylaminopropyl)-N-ethylcarbodiimide hydrochloride (120 mg, 0.61 mmol, 1.2 eq) and tetrahydropyran-4-ylmethyl amine (77 mg, 0.66 mmol, 1.3 eq) were subsequently added to a solution of 6-(3-chloro-phenylamino)-2-trifluoromethyl-nicotinic acid hydrochloride (180 mg, 0.51 mmol, 1.0 eq) in anhydrous DCM (12 mL) and stirred at ambient temperature for 12 h. After evaporation of the solvent in vacuo, the mixture was diluted with ethyl acetate (50 mL) and washed subsequently with a saturated aqueous solution of NaHCO₃ (20 mL×2 times) and brine (25 mL). The organic phase was dried over sodium sulphate and concentrated in vacuo to afford a black residue that was purified by flash chromatography (silica gel, eluent gradient: from hexane/ethyl acetate 1:9 to pure ethyl acetate). The title compound was obtained as a brown solid (130 mg, yield=61%).

EI; TSQ 700; source 180 C; 70 V; 200 uA: 413 (M+.); 315; 299.

¹H NMR (300 MHz, DMSO-d₆) δ: 9.80 (s, 1H); 8.48 (t br, 1H); 8.02 (dd, 1H); 7.72 (d, 1H); 7.51 (dd, 1H); 7.31 (dd, 1H); 7.09 (d, 1H); 7.00 (dd, 1H); 3.89 (m, 2H); 3.27 (m, 2H); 3.09 (dd, 2H); 1.75 (m, 1H); 1.60 (m, 2H); 1.20 (m, 2H).

TABLE 13
Compounds of Example 205 to 209 were prepared as described Example 204, from the
appropriate starting materials via similar intermediates, prepared in a similar manner to
the intermediates described in Descriptions 25 to 29.

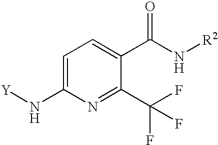

| Ex. | | | | |
|---|---|---|---|---|
| No | Chemical Name | Y | R² | ¹H NMR (Solvent) ppm and/or MS |
| 205 | 6-(3-Chloro- phenylamino)-N- cyclohexylmethyl-2- trifluoromethyl- nicotinamide | 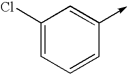 | 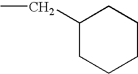 | EI; TSQ 700; source 180 C.; 70 V; 200 uA: 411 (M+), 315, 299. ¹H NMR (300 MHz, DMSO-d₆) δ: 9.80 (s, 1H); 8.38 (t br, 1H); 8.01 (dd, 1H); 7.72 (d, 1H); 7.51 (dd, 1H); 7.32 (dd, 1H); 7.08 (d, 1H); 7.00 (dd, 1H); 3.05 (dd, 2H); 1.77-1.57 (m, 5H); 1.57-1.41 (m, 1H); 1.30-1.10 (m, 3H); 1.02-0.83 (m, 2H). |
| 206 | 6-(3-Chloro- phenylamino)-N- cyclobutylmethyl-2- trifluoromethyl- nicotinamide | 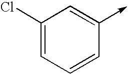 | 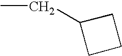 | EI; TSQ 700; source 180 C.; 70 V; 200 uA: 383 (M+); 315; 299. ¹H NMR (300 MHz, DMSO-d₆) δ: 9.80 (s, 1H); 8.40 (t br, 1H); 8.00 (dd, 1H); 7.71 (d, 1H); 7.50 (dd, 1H); 7.30 (dd, 1H); 7.08 (d, 1H); 7.00 (dd, 1H); 3.21 (dd, 2H); 2.50 (m, 1H); 2.00 (m, 2H); 1.95-1.68 (m, 4H). |
| 207 | 6-(3-Chloro- phenylamino)-N- cyclopentylmethyl-2- trifluoromethyl- nicotinamide | 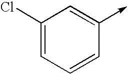 | 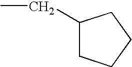 | EI; TSQ 700; source 180 C.; 70 V; 200 uA: 397 (M+); 315; 299. ¹H NMR (300 MHz, DMSO-d₆) δ: 9.80 (s, 1H); 8.42 (t br, 1H); 8.02 (dd, 1H); 7.71 (d, 1H); 7.52 (dd, 1H); 7.33 (dd, 1H); 7.09 (d, 1H); 7.00 (dd, 1H); 3.14 (dd, 2H); 2.08 (m, 1H); 1.76-1.43 (m, 6H); 1.32-1.16 (m, 2H). |

### EXAMPLE 208

### 6-(3-Chloro-phenylamino)-2-isopropyl-N-(tetrahydro-pyran-4-ylmethyl)-nicotinamide

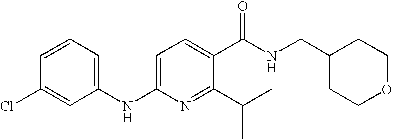

N-methyl morpholine (0.14 mL, 1.27 mmol, 2.5 eq), 1-hydroxy-benzotriazole (100 mg, 0.74 mmol, 1.5 eq), N-(3-dimethylaminopropyl)-N-ethylcarbodiimide hydrochloride (115 mg, 0.6 mmol, 1.2 eq) were subsequently added to a solution of 6-(3-chloro-phenylamino)-2-isopropyl-nicotinic acid hydrochloride (Description 35) (0.16 g, 0.49 mmol, 1.0 eq) in anhydrous DCM (5 mL). After stirring 1 h at room temperature, tetrahydropyran-4-ylmethyl amine (77 mg, 0.66 mmol, 1.3 eq) was added and the resulting solution was stirred at room temperature overnight. Solvent was evaporated in vacuo, the residue was dissolved in ethyl acetate (50 mL) and washed with a saturated aqueous solution of NaHCO₃ and with brine: the organic phase was dried over Na₂SO₄ and concentrated in vacuo to yield a solid that was triturated with hexane/diethyl ether 9:1 and filtered. The title compound was obtained as a white solid (170 mg, yield=89%).

EI; TSQ 700; source 180 C; 70 V; 200 uA: 387 (M+.), 289, 273, 243.

¹H NMR (300 MHz, DMSO-d₆) δ: 9.39 (s, 1H); 8.29 (dd, 1H); 8.21 (t br, 1H); 7.50 (d, 1H); 7.46 (dd, 1H); 7.27 (dd, 1H); 6.91 (dd, 1H); 6.65 (d, 1H); 3.86 (m, 2H); 3.45 (m, 1H); 3.27 (m, 2H); 3.10 (dd, 2H); 1.76 (m, 1H); 1.60 (m, 2H); 1.22 (d, 6H); 1.29-1.12 (m, 2H).

### EXAMPLE 209

### 6-(3-Chloro-phenylamino)-N-(1,1-dioxo-tetrahydrothiophen-3-ylmethyl)-2-isopropyl-nicotinamide

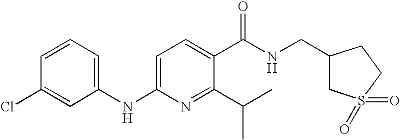

A mixture of 6-(3-chloro-phenylamino)-2-isopropyl-nicotinic acid hydrochloride (Description 35) (166 mg, 0.5 mmol, 1.0 eq), 1-hydroxy-benzotriazole (100 mg, 0.74 mmol, 1.5 eq), PS-dicyclohexylcarbodiimide (760 mg, 1.0 mmol, 2.0 eq, loading=1.31 mmol/g) and PS-diisopropylethylamine (1-54 mg, 0.6 mmol, 1.2 eq, loading=3.88 mmol/g) was stirred at room temperature overnight. The resins were filtered, washed with DCM and tetrahydrofuran (30 mL) and the filtrate was concentrated in vacuo. The residue was dissolved in 2.5 mL of anhydrous THF and C-(1,1-dioxo-tetrahydro-1/⁶-thiophen-3-ylmethyl amine (108 mg, 0.72 mmol, 1.44 eq) and 1-butyl-3-methylimidazolium hexafluorophosphate (53 uL) were then added. The mixture was heated under microwaves irradiation at 140° C. for 20 min, the solvent was removed in vacuo, the residue diluted with ethyl acetate (30 mL) and 5% Na₂CO₃ (aq) (20 mL). The organic phase was then washed with brine (20 mL) and evaporated in vacuo to afford a solid that was purified by flash chromatography (silica gel, eluent: DCM/MeOH/NH₄OH 97:3:0.3). The title compound was obtained as a solid (140 mg, yield=66%).

EI; TSQ 700; source 180 C; 70 V; 200 uA: 421 (M+.); 273.

¹H NMR (300 MHz, DMSO-d₆) δ: 9.41 (s, 1H); 8.36 (t br, 1H); 8.28 (dd, 1H); 7.55 (d, 1H); 7.45 (dd, 1H); 7.27 (dd, 1H); 6.91 (dd, 1H); 6.67 (d, 1H); 3.49-3.15 (m, 5H); 3.07 (m, 1H); 2.85 (dd, 1H); 2.63 (m, 1H); 2.23 (m, 1H); 1.86 (m, 1H); 1.09 (d, 6H).

TABLE 14
All the Examples described in Table 14 were prepared as described for the Example 208
and 209, from the appropriate starting materials via similar intermediates, prepared in a
similar manner to the intermediates described in Descriptions 30 to 35. In particular, the
compounds of the Examples 210 to 214 and 216 to 218 were prepared according to the
same experimental procedure as described for the Example 208, whereas the compounds
of the Examples 215 and 219 were prepared according to the same experimental
procedure as described for the Example 210

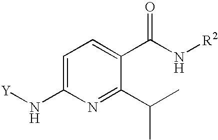

| Ex. | | | | |
|---|---|---|---|---|
| No | Chemical Name | Y | R² | ¹H NMR (Solvent) ppm and/or MS |
| 210 | 6-(3-Chloro- phenylamino)-N- cyclopentylmethyl-2- isopropyl-nicotinamide | 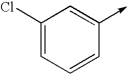 | 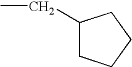 | EI; TSQ 700; source 180 C.; 70 V; 200 uA: 371 (M+.), 289, 273. ¹H NMR (300 MHz, DMSO-d₆) δ: 9.38 (s, 1H); 8.29 (dd, 1H); 8.19 (t br, 1H); 7.48 (d, 1H); 7.45 (dd, 1H); 7.27 dd, 1H; 6.91 dd, 1H; 6.66 d, 1H); 3.44 (m, 1H); 3.13 (dd, 2H); 2.16-2.04 (m, 1H); 1.76-1.42(m, 6H); 1.32-1.19 (m, 2H); 1.22 (d, 6H). |
| 211 | 6-(3-Chloro- phenylamino)-N- cyclohexylmethyl-2- isopropyl-nicotinamide | 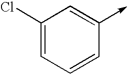 | 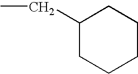 | EI; TSQ 700; source 180 C.; 70 V; 200 uA: 385 (M+.), 289, 273. ¹H NMR (300 MHz, DMSO-d₆) δ: 9.37 (s, 1H); 8.28 (dd, 1H); 8.14 (t br, 1H); 7.49 (d, 1H); 7.46 (dd, 1H); 7.27 (dd, 1H); 6.90 (dd, 1H); 6.65 (d, 1H); 3.45 (m, 1H); 3.05 (dd, 2H); 1.76-1.56 (m, 4H); 1.57-1.43 (m, 1H); 1.22 (d, 6H); |
| # 1.22-1.10 (m, 4H); 0.94 (m, 2H). | | | | |
| 212 | 6-(2,4-Dichloro- phenylamino)-N- cyclobutylmethyl-2- isopropyl-nicotinamide | 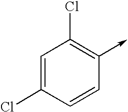 | 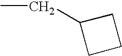 | EI; TSQ 700; source 180 C.; 70 V; 200 uA: 391 (M+.); 356; 322; 307. ¹H NMR (300 MHz, DMSO-d₆) δ: 8.52 (s, 1H); 8.23 (d, 1H); 8.15 (t br, 1H); 7.58 (d, 1H); 7.47 (d, 1H); 7.37 (dd, 1H); 6.86 (d, 1H); 3.39 (m, 1H); 3.23 (dd, 2H); 2.50 (m, 1H); 2.06-1.63 (m, 6H); 1.13 (d, 6H). |
| 213 | 6-(2,4-Dichloro- phenylamino)-N- cyclopentylmethyl-2- isopropyl-nicotinamide | 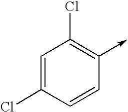 | 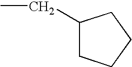 | EI; TSQ 700; source 180 C.; 70 V; 200 uA: 405 (M+.); 370; 307; 288. 1H NMR (300 MHz, DMSO-d₆) δ: 8.53 (s, 1H); 8.23 (d, 1H); 8.19 (t br, 1H); 7.58 (d, 1H); 7.48 (d, 1H); 7.37 (dd, 1H); 6.87 (d, 1H); 3.39 (m, 1H); 3.13 (dd, 2H); 2.11 (m, 1H); 1.75-1.41 (m, 6H); 1.23 (m, 2H); 1.14 (d, 6H). |
| 214 | 6-(2,4-Dichloro- phenylamino)-N- (tetrahydro-pyran-4- ylmethyl)-2-isopropyl- nicotinamide | 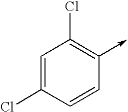 | 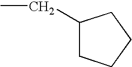 | EI; TSQ 700; source 180 C.; 70 V; 200 uA: 421 (M+.); 386; 307; 288. 271. ¹H NMR (300 MHz, DMSO-d₆) δ: 8.53 (s, 1H); 8.23 (d, 1H); 8.20 (t br, 1H); 7.58 (d, 1H); 7.51 (d, 1H); 7.37 (dd, 1H); 6.87 (d, 1H); 3.85 (m, 2H); 3.39 (m, 1H); 3.26 (m, 2H); 3.10 (dd, 2H); 1.75 (m, 1H); 1.60 (m, 2H); |
| # 1.28-1.07 (m, 2H); 1.13 (d, 6H). | | | | |
| 215 | 6-(2,4-Dichloro- phenylamino)-N-(1,1- tetrahydrothiophen-3- ylmethyl)-2-isopropyl- nicotinamide | 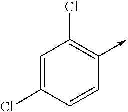 | 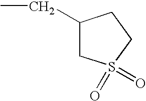 | EI; TSQ 700; source 180 C.; 70 V; 200 uA: 455 (M+.), 420, 307. ¹H NMR (300 MHz, DMSO-d₆) δ: 1H); 7.32 (d, 1H); 6.78 (d, 1H); 3.40-3.10 (m, 5H); 3.04 (m, 1H); 2.80 (dd, 1H); 2.63 (m, 1H); 2.23 (m, 1H); 1.82 (m, 1H); 1.09 (d, 6H). |
| 216 | 6-(3-Fluoro- phenylamino)-N- cyclobutylmethyl-2- isopropyl-nicotinamide | 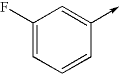 | 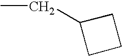 | EI; TSQ 700; source 180 C.; 70 V; 200 uA: 341 (M+.); 257. ¹H NMR (300 MHz, DMSO-d₆) δ: 9.38 (s, 1H); 8.15 (t br, 1H); 8.00 (d, 1H); 7.46 (d, 1H); 7.34-7.21 (m, 2H); 6.67 (m, 1H); 6.65 (d, 1H); 3.44 (m, 1H); 3.23 (dd, 2H); 2.50 (m, 1H); 2.07-1.64 (m, 6H); 1.21 (d, 6H). |
| 217 | 6-(3-Fluoro- phenylamino)-N- cyclopentylmethyl-2- isopropyl-nicotinamide | 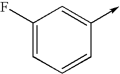 | 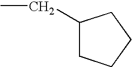 | EI; TSQ 700; source 180 C.; 70 V; 200 uA: 355 (M+.); 273; 257; 227. ¹H NMR (300 MHz, DMSO-d₆) δ: 9.38 (s, 1H); 8.19 (t br, 1H); 8.01 (ddd, 1H); 7.47 (d, 1H); 7.34-7.22 (m, 2H); 6.67 (m, 1H); 6.66 (d, 1H); 3.44 (m, 1H); 3.14 (dd, 2H); 2.11 (m, 1H); 1.76-1.43 (m, 6H); 1.25 (m, 2H); 1.22 (d, 6H). |
| 218 | 6-(3-Fluoro- phenylamino)-N- (tetrahydro-pyran-4- ylmethyl)-2-isopropyl- nicotinamide | 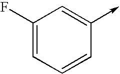 | 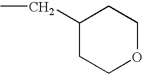 | EI; TSQ 700; source 180 C.; 70 V; 200 uA: 371 (M+.); 273; 257; 227. ¹H NMR (300 MHz, DMSO-d₆) δ: 9.39 (s, 1H); 8.20 (t br, 1H); 8.00 (d, 1H); 7.50 (d, 1H); 7.34-7.20 (m, 2H); 6.67 (m, 1H); 6.66 (d, 1H); 3.84 (m, 2H); 3.45 (m, 1H); 3.36-3.00 (m, 2H); 3.11 (dd, 2H); 1.76 (m, 1H); 1.61 |
| # (m, 2H); 1.33-1.04 (m, 2H); 1.21 (d, 6H). | | | | |
| 219 | 6-(3-Fluoro- phenylamino)-N-(1,1- dioxo- tetrahydrothiophen-3- ylmethyl)-2-isopropyl- nicotinamide | 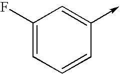 | 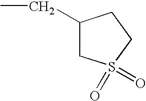 | ESI POS, spray 3.5 KV/source: 30 V/PROBE: 250 C.: 406 (MH+). ¹H NMR (300 MHz, DMSO-d₆) δ: 9.44 (s, 1H); 8.36 (t br, 1H); 8.00 (ddd, 1H); 7.55 (d, 1H); 7.35-7.22 (m, 2H); 6.68 (m, 1H); 6.67 (d, 1H); 3.35-3.14 (m, 5H); 3.07 (m, 1H); 2.85 (dd, 1H); 2.64 (m, 1H); 2.23 (m, 1H); |
| # 1.86 (m, 1H); 1.22 (d, 6H). | | | | |

### EXAMPLE 220

### 6-(4-Cyano-2-methyl-phenylamino)-4-isopropyl-N-(tetrahydro-pyran-4-ylmethyl)-nicotinamide

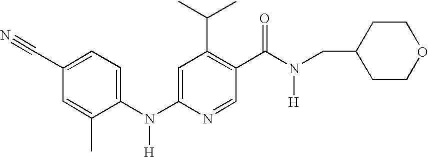

A mixture of 6-chloro-4-isopropyl-N-(tetrahydro-pyran-4-ylmethyl)-nicotinamide (Description 8) (100 mg), 4-amino-3-methyl benzonitrile (2eq), cesium carbonate (168 mg), tris(dibenzylideneacetone)palladium(0) (Pd₂(dba)₃) (3.4 mg), 4,5-bis(diphenylphosphino)-9,9-dimethylxanthene (Xantphos) (2.3 mg) in 1,4-dioxane (1 ml) was irradiated under microwave conditions at 150° C. for 30 minutes. Further quantities of cesium carbonate (168 mg), Pd₂(dba)₃ (3.4 mg) and Xantphos (2.3 mg) were added and the mixture was again subjected to microwave conditions at 150° C. for 30 minutes. Ethyl acetate was added and the mixture was washed with water. The ethyl acetate layer was dried (sodium sulphate) and the solvent was removed under reduced pressure. The residue was purified using MDAP to give the title compound (20 mg)

NMR (MeOD) δ 1.25 (6H, d), 1.29-1.43 (2H, m), 1.70 (2H, d), 1.81-1.93 (1H, m), 2.3393H, s), 3.21-3.50 (5H, m), 3.98 (2H, dd), 7.01 (1H, s), 7.49 (1H, dd), 7.55 (1H, bs), 8.02 (1H, d), 8.09 (1H, s)

LC/MS, t=2.89 min, Molecular ion observed [MH⁺]=393 consistent with the molecular formula C₂₃H₂₈N₄O₂

### EXAMPLE 221

### 6-(S-Chloro-2-cyano-phenylamino)-4-isopropyl-N-(tetrahydro-pyran-4-ylmethyl)-nicotinamide

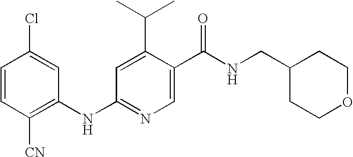

A mixture of 6-chloro-4 isopropyl-N-(tetrahydro-pyran-4-ylmethyl)-nicotinamide (Description 8) (100 mg), 2-amino-4-chlorobenzonitrile (61 mg), cesium carbonate (154 mg), tris(dibenzylideneacetone)palladium(0) (3.2 mg), 4,5-bis(diphenylphosphino)-9,9-dimethyl xanthene (Xantphos) (2.2 mg) and dioxan (1 ml) was stirred under reflux under nitrogen for 24 hours. The mixture was allowed to cool and insoluble material filtered and washed with ethyl acetate. The filtrate was evaporated under reduced pressure and the residue purified by trituration with ether followed by recrystallisation from methanol to give the title compound as a yellow solid (53 mg).

NMR (DMSO-d6) δ 1.2-1.3 (2H, m), 1.21 (6H, d), 1.62 (2H, d), 1.77 (1H, m), 3.15 (2H, t), 3.29 (2H, t), 3.33 (1H, m), 3.86 (2H, d), 7.05 (1H, s), 7.36 (1H, d), 7.46 (1H, s), 8.36 (1H, d), 8.79 (1H, t), 9.00 (1H, s), 9.74 (1H, s).

LC/MS t=2.3 min, [MH⁺] 413 consistent with the molecular formula C₂₂H³⁵ClN₄O₂.

### EXAMPLE 222

### 6-(2-cyano-5-methyl-phenylamino)-4-isopropyl-N-(tetrahydro-pyran-4-ylmethyl)-nicotinamide

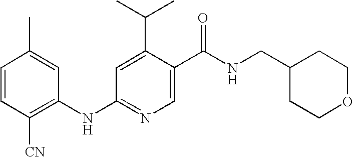

In a manner similar to Example 221, 6-chloro-4-isopropyl-N-(tetrahydro-pyran-4-ylmethyl)-nicotinamide (Description 8) (100 mg) and 2-amino-4-methylbenzonitrile (44.5 mg) afforded the title compound (38 mg).

LC/MS t=1.9 min, [MH⁺] 393 consistent with the molecular formula C₂₃H₂₈N₄O₂.

### EXAMPLE 223

### 6-(3-Chloro-phenylamino)-N-(1,1-dioxo-tetrahydro-1/⁶-thiophen-3-ylmethyl)-4-isopropyl-nicotinamide

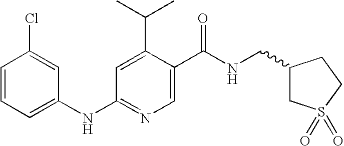

In a manner similar to that described in Example 50, 6-(3-chloro-phenylamino)-4 isopropyl-nicotinic acid (Description 13) (30 mg) and C-(1,1-dioxo-tetrahydro-1/⁶-thiophen-3-yl)-methylamine hydrochloride (Argyle et al, J Chem Soc (C), 1967, 2156) (23 mg) afforded the title compound (32 mg).

LC/MS t=3.0 min, [MH⁺] 422 consistent with C₂₀H₂₄³⁵ClN₃O₃S

### EXAMPLE 224

### N-Cyclobutylmethyl-4-isopropyl-6-(3-trifluoromethoxy-phenylamino)-nicotinamide

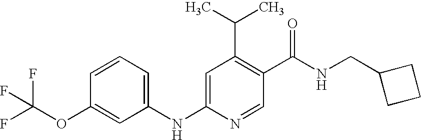

In a manner similar to Example 6,6-chloro-N-cyclobutylmethyl-4-isopropyl-nicotinamide (Description 6) (80 mg) and 3-trifluoromethoxyaniline (0.5 ml) gave the title compound (41 mg).

LC/MS, t=3.73 min, Molecular ion observed [MH⁺]=408 consistent with the molecular formula C₂₁H₂₄F₃N₃O₂

TABLE 15
Examples 225 to 233 were prepared by the method given in column 4 and purified by the
procedure given in column 5

| | | | | | 1. Retention time |
|---|---|---|---|---|---|
| | | | | | (min). |
| Example | | | Preparation | Purification | 2. [MH+] |
| no | Compound Name | Compound Structure | method | Method | 3. Molecular Formula |
| 225 | 6-(2,3-Difluoro-phenylamino)-4- isopropyl-N-(tetrahydro-pyran- 4-ylmethyl)-nicotinamide | 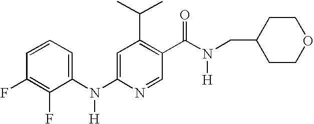 | G | E | 2.82 min 390 C₂₁H₂₅F₂N₃O₂ |
| 226 | 6-(3,5-Bis-trifluoromethyl- phenylamino)-4-isopropyl-N- (tetrahydro-pyran-4-ylmethyl)- nicotinamide | 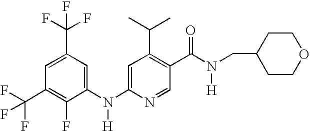 | G | E | 3.60 min 490 C₂₃H₂₅F₆N₃O₂ |
| 227 | 6-(2,4-Difiuoro-phenylamino)-4- isopropyl-N-(tetrahydro-pyran- 4-ylmethyl)-nicotinamide | 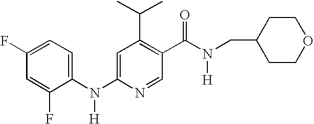 | G | E | 2.70 min 390 C₂₁H₂₅F₂N₃O₂ |
| 228 | 6-(3-Ethynyl-phenylamino)-4- isopropyl-N-(tetrahydro-pyran- 4-ylmethyl)-nicotinamide | 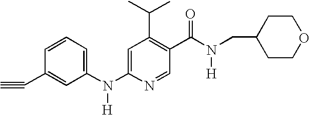 | G | E | 2.88 min 378 C₂₃H₂₇N₃O₂ |
| 229 | 6-(2-Fluoro-4-trifluoromethyl- phenylamino)-N- cyclopentylmethyl-4-isopropyl- nicotinamide | 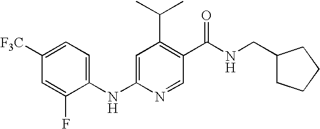 | G | E | 3.82 424 C₂₂H₂₅F₄N₃O |
| 230 | 6-(3-cyano-4-methyl- phenylamino)-4-isopropyl-N- (tetrahydropyran-4-ylmethyl)- nicotinamide | 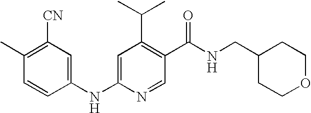 | J | H | 2.90 393 C₂₃H₂₈N₄O₂ |
| 231 | 6-(3-cyano-4-fluoro- phenylamino)- 4-isopropyl-N- (tetrahydropyran-4-ylmethyl)- nicotinamide | 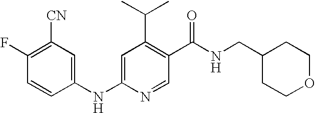 | J | Ethyl acetate trituration of crude product | 2.80 397 C₂₂H₂₅FN₄O₂ |
| 232 | 6-(3-bromo-4-trifluoromethoxy- phenylamino)-4-isopropyl-N- (tetrahydropyran-4-ylmethyl)- nicotinamide | 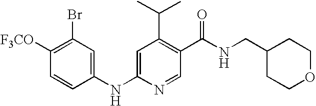 | J | Ether trituration of the crude product | 3.60 516 C₂₂H₂₅⁷⁹Br F₃N₃O₃ |
| 233 | 6-(4-Chloro-2-fluoro- phenylamino)-N- cyclobutylmethyl-4-isopropyl- nicotinamide | 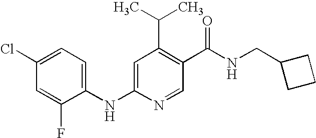 | J | H | 3.58 376 C₂₀H₂₃³⁵ClFN₃O |

Preparation method G: As for the preparation of Example 75
Preparation method J: As for the preparation of Example 46
Purification method E: Mass-directed autopreparative technique
Purification method H: Biotage Horizon

TABLE 16
Examples 234 to 279 in this table were prepared by the method and reaction time given in column 4 and
purified by the procedure given in column 5.

| | | | Method/ | | RT (min), (MH+) |
|---|---|---|---|---|---|
| Example | | | Reaction | Purification | Consistent with |
| No. | Name | Structure | Time | Method | molecular formula |
| 234 | 6-(5-Bromo-2-methyl- phenylamino)-4-isopropyl-N- (tetrahydro-pyran-4-ylmethyl)- nicotinamide | 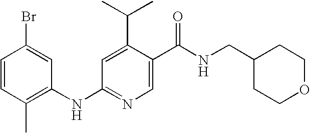 | G 30 min | E | 3.0 446 C₂₂H₂₈⁷⁹BrN₃O₂ |
| 235 | 6-(2-Bromo-5-fluoro- phenylamino)-4-isopropyl-N- (tetrahydro-pyran-4-ylmethyl)- nicotinamide | 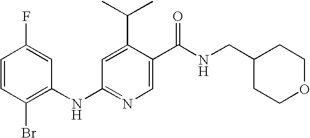 | G 1 hour | E | 3.0 450 C₂₁H₂₅⁷⁹BrFN₃O₂ |
| 236 | 6-(2-Fluoro-5-trifluoromethyl- phenylamino)-4-isopropyl-N- (tetrahydro-pyran-4-ylmethyl)- nicotinamide | 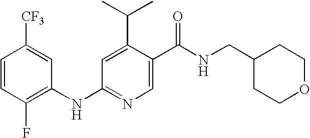 | G 30 min | E | 3.2 440 C₂₂H₂₅F₄N₃O₂ |
| 237 | 6-(2-Chloro-5-trifluoromethyl- phenylamino)-4-isopropyl-N- (tetrahydro-pyran-4-ylmethyl)- nicotinamide | 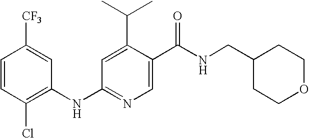 | G 1 hour | E | 3.4 456 C₂₂H₂₅³⁵ClF₃N₃O₂ |
| 238 | 6-(2-Bromo-5-trifluoromethyl- phenytamino)-4-isopropyl-N- (tetrahydro-pyran-4-ylmethyl)- nicotinamide | 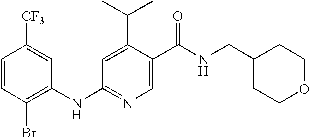 | G 1 hour | E | 3.4 500 C₂₂H₂₅⁷⁹BrF₃N₃O₂ |
| 239 | 6-(3-Bromo-4-cyano- phenylamino)-4-isopropyl-N- (tetrahydro-pyran-4-ylmethyl)- nicotinamide | 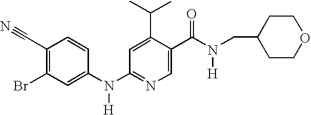 | G 30 min | E | 3.10 459 C₂₂H₂₅⁸¹BrN₄O₂ |
| 240 | 6-(2-Bromo-4-trifluoromethoxy- phenylamino)-4-isopropyl-N- (tetrahydro-pyran-4-ylmethyl)- nicotinamide | 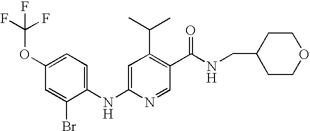 | G 30 min | E | 3.40 518 C₂₂H₂₅⁸¹BrF₃N₃O₃ |
| 241 | 6-(3-Chloro-2-methyl- phenylamino)-4-isopropyl-N- (tetrahydro-pyran-4-ylmethyl)- nicotinamide | 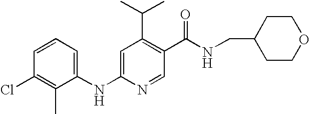 | G 30 min | E | 2.29 402 C₂₂H₂₈³⁵ClN₃O₂ |
| 242 | 6-(3,5-Difluoro-phenylamino)-4- isopropyl-N-(tetrahydro-pyran-4- ylmethyl)-nicotinamide | 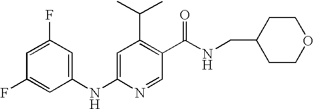 | G 30 min | E | 3.06 390 C₂₁H₂₅F₂N₃O₂ |
| 243 | 6-(2-Chloro-4-fluoro- phenylamino)-4-isopropyl-N- (tetrahydro-pyran-4-ylmethyl)- nicotinamide | 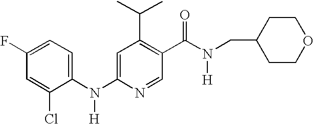 | G 30 min | E | 2.86 406 C₂₁H₂₅³⁵ClFN₃O₂ |
| 244 | 6-(4-Chloro-2-methyl- phenylamino)-4-isopropyl-N- (tetrahydro-pyran-4-ylmethyl)- nicotinamide | 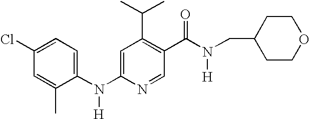 | G 30 min | E | 2.90 402 C₂₂H₂₈³⁵ClN₃O₂ |
| 245 | 6-(2-Fluoro-3-trifluoromethyl- phenylamino)-N- cyclopentylmethyl-4-isopropyl- nicotinamide | 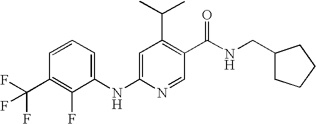 | G 30 Min | H | 3.72 424 C₂₂H₂₅F₄N₃O |
| 246 | 6-(2-Methyl-4-chloro- phenylamino)-N- cyclopentylmethyl-4-isopropyl- nicotinamide | 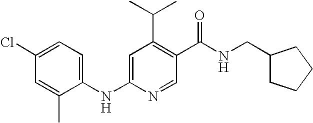 | G 30 Min | H | 3.50 386 C₂₂H₂₈³⁵ClN₃O |
| 247 | 6-(3-Chloro-4-cyano- phenytamino)-N- cyclopentylmethyl-4-isopropyl- nicotinamide | 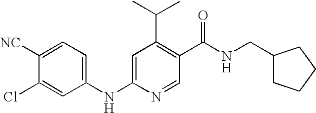 | G 30 Min | H | 3.68 397 C₂₂H₂₅³⁵ClN₄O |
| 248 | 6-(4-bromo-2-chloro phenylamino)-N- cyclopentylmethyl-4-isopropyl- nicotinamide | 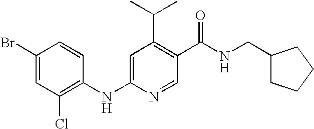 | G 30 Min | E | 3.91 450 C₂₂H₂₅⁷⁹Br³⁵ClN₃O |
| 249 | N-Cyclobutylmethyl-6-(2,4- difluoro-phenylamino)-4- isopropyl-nicotinamide | 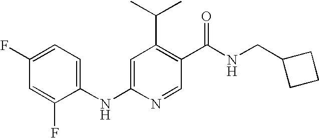 | G 1 hour | F | 3.24 360 C₂₀H₂₃F₂N₃O |
| 250 | N-Cyclobutylmethyl-6-(2,4- dichloro-phenylamino)-4- isopropyl-nicotinamide | 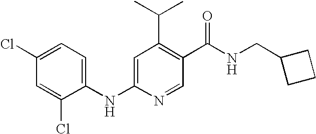 | G 1 hour | F | 3.75 392 C₂₀H₂₃³⁵Cl₂N₃O |
| 251 | N-Cyclobutylmethyl-6-(3,4- dichtoro-phenylamino)-4- isopropyl-nicotinamide | 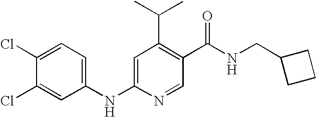 | G 1 hour | Crude product purified by trituration with 1:1 DCM/Ether | 3.89 392 C₂₀H₂₃³⁵Cl₂N₃O |
| 252 | N-Cyclobutylmethyl-6-(2,3- dichloro-phenylamino)-4- isopropyl-nicotinamide | 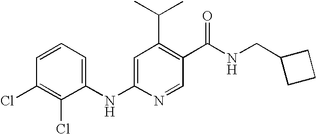 | G 1 hour | H | 3.68 392 C₂₀H₂₃³⁵Cl₂N₃O |
| 253 | 6-(2-Chloro-4-fluoro- phenylamino)-N- cyclobutylmethyl-4-isopropyl- nicotinamide | 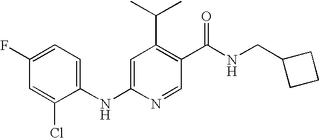 | G 1 hour | F | 3.37 376 C₂₀H₂₃³⁵ClFN₃O |
| 254 | 6-(3-Chloro-4-fluoro- phenylamino)-N- cyclobutylmethyl-4-isopropyl- nicotinamide | 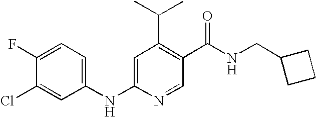 | G 1 hour | H | 3.63 376 C₂₀H₂₃³⁵ClFN₃O |
| 255 | 6-(4-Bromo-2-chloro- phenylamino)-N- cyclobutylmethyl-4-isopropyl- nicotinamide | 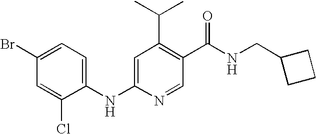 | G 1 hour | H | 3.81 436 C₂₀H₂₃⁷⁹Br₃₅ClN₃O |
| 256 | 6-(2-Bromo-4-chloro- phenylamino)-N- cyclobutylmethyl-4-isopropyl- nicotinamide | 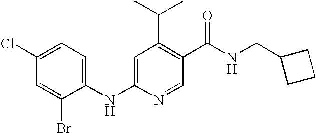 | G 1 hour | H | 3.75 436 C₂₀H₂₃⁷⁹Br₃₅ClN₃O |
| 257 | N-cyclobutylmethyl-6-(2-fluoro-3- trifluoromethyl-phenylamino)-4- isopropyl-nicotinamide | 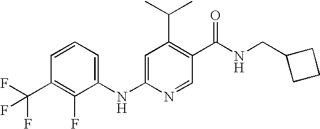 | G 1 hour | H | 3.64 410 C₂₁H₂₃F₄N₃O |
| 258 | 6-(4-Chloro-2-methyl- phenylamino)-N- cyclobutylmethyl-4-isopropyl- nicotinamide | 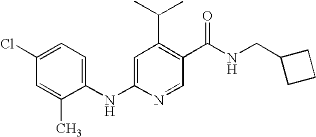 | G 1 hour | H | 3.35 372 C₂₁H₂₆³⁵ClN₃O |
| 259 | 6-(2-Chloro-4-cyano- phenylamino)-N- cyclobutylmethyl-4-isopropyl- nicotinamide | 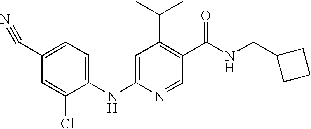 | K 3 hours | L | 3.41 383 C₂₁H₂₃³⁵ClN₄O |
| 260 | 6-(4-Cyano-2-fluoro- phenylamino)-N- cyclobutylmethyl-4-isopropyl- nicotinamide | 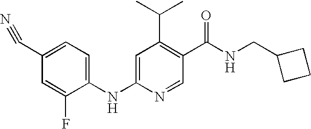 | K 4 hours | L | 3.32 367 C₂₁H₂₃FN₄O |
| 261 | 6-(4-Cyano-2-methyl- phenylamino)-N- cyclobutylmethyl-4-isopropyl- nicotinamide | 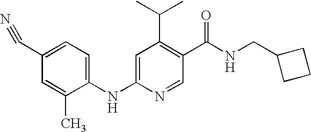 | K 4 hours | L | 3.24 363 C₂₂H₂₆N₄O |
| 262 | 6-(2-Chloro-4-trifluoromethyl- phenylamino)-N- cyclobutylmethyl-4-isopropyl- nicotinamide | 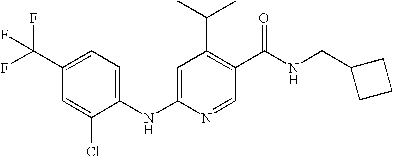 | K 4 hours | E | 3.86 426 C₂₁H₂₃³⁵ClF₃N₃O |
| 263 | N-Cyclobutylmethyl-6-(3,5- dichloro-phenylamino)-4- isopropyl-nicotinamide | 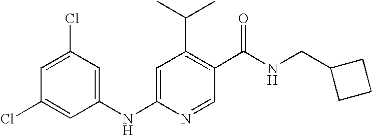 | G 1 hour | H | 4.01 392 C₂₀H₂₃³⁵Cl₂N₃O |
| 264 | N-Cyclobutylmethyl-6-(2,5- dichloro-phenylamino)-4- isopropyl-nicotinamide | 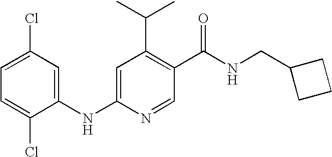 | G 1 hour | H | 3.78 392 C₂₀H₂₃³⁵Cl₂N₃O |
| 265 | N-Cyclobutylmethyl-6-(3,5- difluoro-phenylamino)-4- isopropyl-nicotinamide | 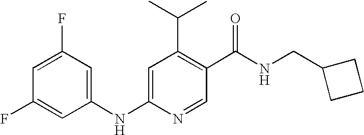 | G 1 hour | H | 3.57 360 C₂₀H₂₃F₂N₃O |
| 266 | 6-(5-Chloro-2-fluoro- phenylamino)-N- cyclobutylmethyl-4-isopropyl- nicotinamide | 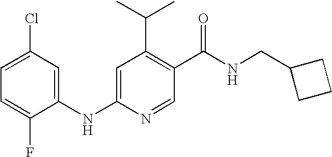 | G 1 hour | H | 3.62 376 C₂₀H₂₃³⁵ClFN₃O |
| 267 | 6-(2-Chloro-5-fluoro- phenylamino)-N- cyclobutytmethyl-4-isopropyl- nicotinamide | 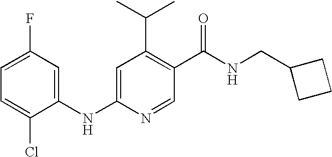 | G 1 hour | H | 3.56 376 C₂₀H₂₃³⁵ClFN₃O |
| 268 | 6-(3-Chloro-phenylamino)-N- isobutyl-4-isopropyl-nicotinamide | 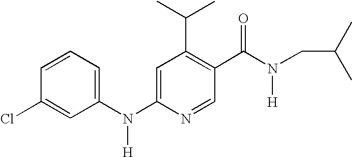 | G 30 min | H | 3.28 346 C₁₉H₂₄³⁵ClN₃O |
| 269 | N-Isobutyl-4-isopropyl-6-(3- trifluoromethyl-phenylamlno)- nicotinamide | 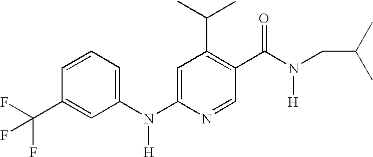 | G 30 min | H | 3.53 380 C₂₀H₂₄F₃N₃O |
| 270 | 6-(3,4-Dichloro-phenylamino)-N- isobutyl-4-isopropyl-nicotinamide | 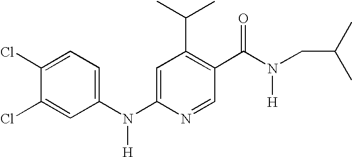 | G 30 min | H | 3.72 380 C₁₉H₂₃³⁵Cl₂N₃O |
| 271 | 6-(2-Fluoro-3-trifluoromethyl- phenylamino)-N-isobutyl-4- isopropyl-nicotinamide | 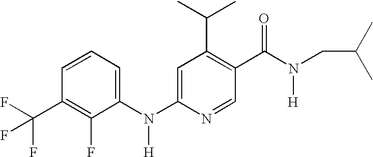 | G 30 min | H | 3.37 398 C₂₀H₂₃F₄N₃O |
| 272 | 6-(3-Bromo-2-methyl- phenylamino)-N-isobutyl-4- isopropyl-nicotinamide | 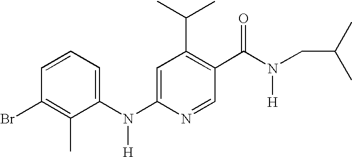 | G 30 min | H | 3.44 406 C₂₀H₂₆⁸¹BrN₃O |
| 273 | 6-(2,4-Dichloro-phenylamtno)-N- isobutyl-4-isopropyl-nicotinamide | 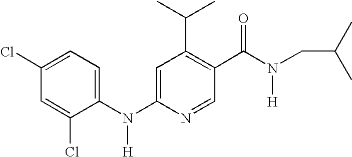 | G 30 min | H | 3.70 380 C₁₉H₂₃³⁵Cl₂N₃O |
| 274 | 6-(2-Chloro-5-fluoro- phenylamino)-N-isobutyl-4- isopropyl-nicotinamide | 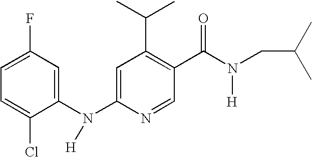 | G 30 min | H | 3.60 364 C₁₉H₂₃ClFN₃O |
| 275 | 6-(3,5-Difluoro-phenylamino)-N- isobutyl-4-isopropyl-nicotinamide | 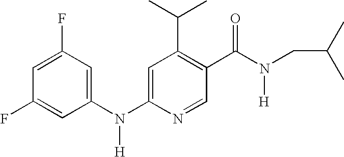 | G 30 min | H | 3.56 348 C₁₉H₂₃F₂N₃O |
| 276 | 6-(5-Chloro-2-fluoro- phenylamino)-N-isobutyl-4- isopropyl-nicotinamide | 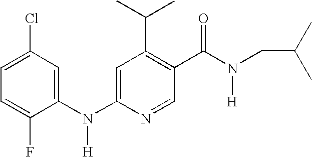 | G 30 min | H | 3.60 364 C₁₉H₂₃ClFN₃O |
| 277 | 6-(3-Bromo-phenylamino)-N- isobutyl-4-isopropyl-nicotinamide | 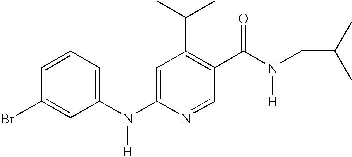 | G 30 min | H | 3.63 392 C₁₉H₂₄BrN₃O |
| 278 | 6-(2,4-Dichloro-phenylamino)-N- (1,1-dioxo-tetrahydro-1/⁶- thiophen-3-ylmethyl)-4-isopropyl- nicotinamide | 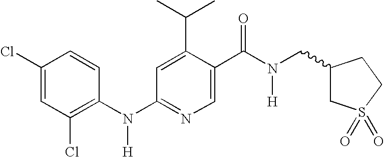 | G 30 min From Description 38 | E | 3.2 456 C₂₀H₂₃³⁵Cl₂N₃O₃S |
| 279 | 6-(4-Bromo-3-fluoro- phenylamino)-N-(1,1-dioxo- tetrahydro-1/⁶-thiophen-3- ylmethyl)-4-isopropyl- nicotinamide | 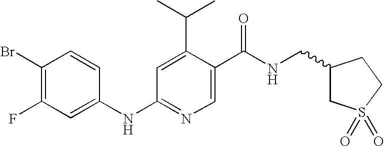 | G 30 min From Description 38 | E | 3.2 484 C₂₀H₂₃⁷⁹BrFN₃O₃S |

Method G: Examples were prepared as for Example 75
Method K: Examples were prepared as for Example 221.
Purification method E: mass-directed auto-preparative technique
Purification method H: Biotage Horizon
Purification method L: the reaction was evaporated, taken up in 1:1 DCM/MeOH, filtered, evaporated, and the residue triturated with MeOH

TABLE 17
Examples in this table were prepared by the method and reaction time given
in column 4 and purified by the procedure given in column 5.

| | | | Method/ | | RT (min), (MH+) |
|---|---|---|---|---|---|
| Example | | | Reaction | Purification | Consistent with |
| No. | Name | Structure | Time | Method | molecular formula |
| 280 | 6-(2-Chloro-5- fluoro- phenylamino)-4- isopropyl-N- (tetrahydro- pyran-4- ylmethyl)- nicotinamide | 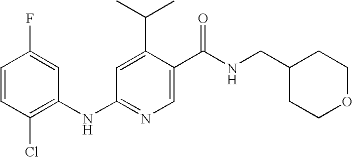 | G 1 hour | E | 3.1 406 C₂₁H₂₅³⁵ClFN₃O₂ |
| 281 | 6-(2-Chloro-5- methyl- phenylamino)-4- isopropyl-N- (tetrahydro- pyran-4- ylmethyl)- nicotinamide | 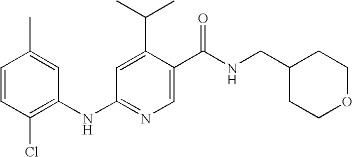 | G 30 min | E | 3.0 402 C₂₂H₂₈³⁵ClN₃O₂ |
| 282 | 6-(2-Fluoro-5- methyl- phenylamino)-4- isopropyl-N- (tetrahydro- pyran-4- ylmethyl)- nicotinamide | 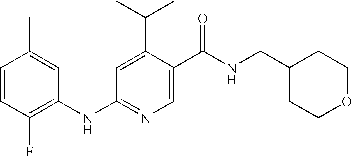 | G 30 min | E | 2.8 386 C₂₂H₂₈FN₃O₂ |
| 283 | 6-(5-Fluoro-2- methyl- phenylamino)-4- isopropyl-N- (tetrahydro- pyran-4- ylmethyl)- nicotinamide | 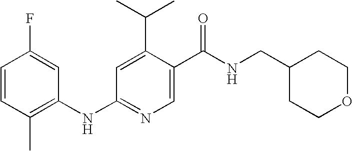 | G 1 hour | E | 2.7 386 C₂₂H₂₈FN₃O₂ |
| 284 | 6-(3-Bromo-2- methyl- phenylamino)-4- isopropyl-N- (tetrahydro- pyran-4- ylmethyl)- nicotinamide | 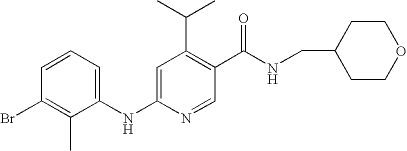 | G 30 min | E | 2.98 448 C₂₂H₂₈⁸¹BrN₃O₂ |
| 285 | 4-Isopropyl-6- (2-methyl-4- trifluoromethoxy- phenylamino)- N-(tetrahydro- pyran-4- ylmethyl)-nicotinamide | 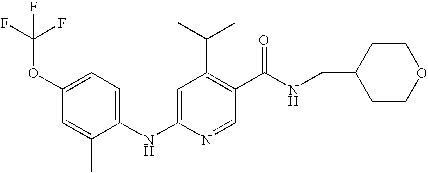 | G 30 min | E | 3.14 452 C₂₃H₂₈F₃N₃O₃ |
| 286 | 6-(3-Fluoro-2- methyl- phenylamino)-4- isopropyl-N- (tetrahydro-pyran- 4-ylmethyl)- nicotinamide | 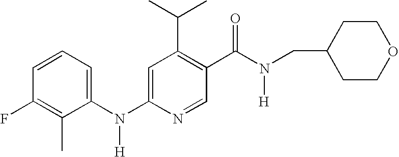 | G 30 min | E | 2.70 386 C₂₂H₂₈FN₃O₂ |
| 287 | 6-(3-Bromo-5- trifluoromethyl- phenylamino)-4- isopropyl-N- (tetrahydro-pyran- 4-ylmethyl)- nicotinamide | 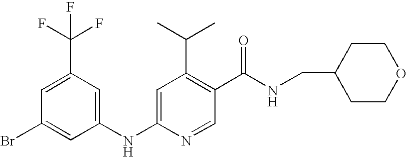 | G 30 min | E | 3.59 501 C₂₂H₂₅⁸¹BrF₃N₃O₂ |
| 288 | 6-(4-Cyano- phenylamino)-4- isopropyl-N- (tetrahydro-pyran- 4-ylmethyl)- nicotinamide | 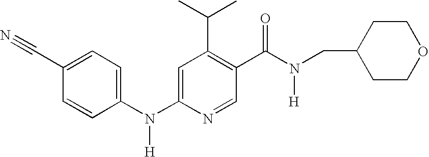 | G 30 min | E | 3.60 490 C₂₂H₂₆N₄O₂ |
| 289 | 6-(4-Chloro-2- fluoro- phenylamino)-4- isopropyl-N- (tetrahydro-pyran-4-ylmethyl)- nicotinamide | 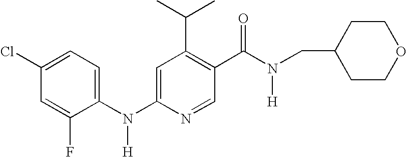 | G 30 min | E | 2.72 379 C₂₁H₂₅³⁵ClFN₃O₂ |
| 290 | 6-(4-bromo-2- fluoro- phenylamino)-N- cyclopentylmethyl-4-isopropyl- nicotinamide | 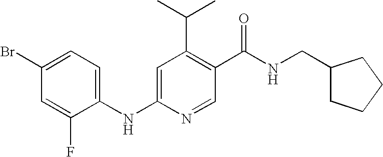 | G 30 min | E | 3.75 434 C₂₁H₂₅⁷⁹BrFN₃O |
| 291 | 6-(2-Bromo-4- trifluoromethoxy- phenylamino)-N- cyclobutylmethyl-4-isopropyl- nicotinamide | 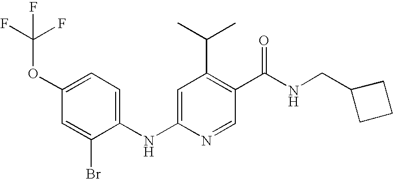 | G 1 hour | H | 3.84 486 C₂₁H₂₃⁷⁹BrF₃N₃O₂ |
| 292 | N-Cyclobutyl- methyl-6- (2-fluoro- 4-trifluoromethyl- phenylamino)- 4-isopropyl-nicotinamide | 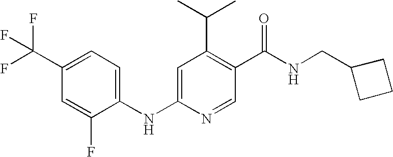 | K 8 hours | E | 3.71 410 C₂₁H₂₃F₄N₃O |

Method G: Examples were prepared as for example 75
Method K: Examples were prepared as for Example 221.
Purification method E: mass-directed auto-preparative technique
Purification method H: Biotage Horizon

### EXAMPLE 293

### 6-(4-Cyano-2-fluoro-phenylamino)-N-cyclopentylmethyl-4-isopropyl-nicotinamide

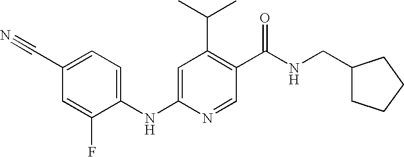

Prepared in a manner similar to Example 221 from 6-chloro-N-cyclopentylmethyl-4-isopropyl-nicotinamide (Description 10) and 4-cyano-2-fluoro-aniline, to give the title compound (16 mg).

NMR (DMSO-d6) δ1.16 (6H, d), 1.23 (2H, m), 1.51-1.68 (6H, m), 2.11 (1H, m), 3.17 (2H, s), 4.11 (1H, s), 7.25 (1H, s), 7.61 (1H, d), 7.80 (1H, d), 8.12 (1H, s), 8.43 (1H, s), 8.72 (1H, t), 9.37 (1H, s).

LC/MS t=3.4 min, [MH⁺] 381, consistent with molecular formula C₂₂H₂₅FN₄O

### EXAMPLE 294

### 6-(4-Bromo-3-trifluoromethyl-phenylamino)-4-isopropyl-N-(tetrahydropyran-4-ylmethyl)-nicotinamide

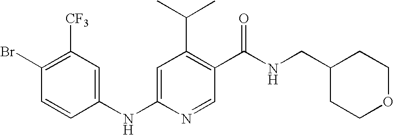

A mixture of 6-chloro-4-isopropyl-N-(tetrahydropyran-4-ylmethyl)-nicotinamide (Description 8) (100 mg), 4-bromo-3-trifluoromethyl- (ex Lancaster, 162 mg), methanesulfonic acid (44 μl) in 1,4-dioxane (1 ml) was irradiated under microwave conditions at 180° for 30 minutes. After removal of the 1,4-dioxane under reduced pressure, the mixture was partitioned between ethyl acetate (5 ml) and brine (2 ml) and the aqueous layer separated. The organic layer was evaporated under reduced pressure and the residue purified using the Biotage Horizon system. Purification afforded the title compound as a white solid (47 mg).

NMR (DMSO-d6) δ1.16-1.23 (8H, d, m), 1.60-1.63 (2H, d), 1.75 (1H, m), 3.10 (2H, t), 3.28 (2H, t), 3.41 (1H, m), 3.85 (2H, d), 6.80 (1H, s), 7.73 (1H, d), 7.83 (1H, d), 8.16 (1H, s), 8.38-8.42 (2H, m), 9.70 (1H, s).

LC/MS t=3.5 min, [MH⁺] 500, consistent with molecular formula C₂₂H₂₅⁷⁹BrF₃N₃O₂

### EXAMPLE 295

### 6-(4-Fluoro-3-trifluoromethyl-phenylamino)-4-isopropyl-N-(tetrahydropyran-4-ylmethyl)-nicotinamide

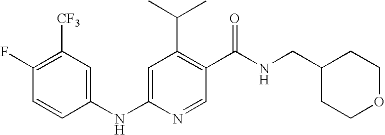

Prepared in a manner similar to Example 294 from 6-chloro-4-isopropyl-N-(tetrahydropyran-4-ylmethyl)-nicotinamide (Description 8) and 4-fluoro-3-trifluoromethyl-aniline (ex Lancaster, 120 mg). Purified by trituration with ether to afford the title compound as a white solid (121 mg).

NMR (DMSO-d6) δ 1.09-1.24 (8H, d, m), 1.60-1.63 (2H, d), 1.76 (1H, m), 3.10 (2H, t), 3.28 (2H, t), 3.41 (1H, m), 3.85 (2H, d), 6.78 (1H, s), 7.42 (1H, t), 7.86 (1H, d), 8.13 (1H, s), 8.30 (1H, d), 8.40 (1H, t), 9.60 (1H, s).

LC/MS t=3.3 min, [MH⁺] 440, consistent with molecular formula C₂₂H₂₅F₄N₃O₂

### EXAMPLE 296

### 6-(3,4-Dibromo-phenylamino)-4-isopropyl-N-(tetrahydropyran-4-ylmethyl)-nicotinamide

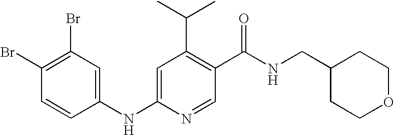

Prepared in a manner similar to Example 294 from 6-chloro-4-isopropyl-N-(tetrahydropyran-4-ylmethyl)-nicotinamide (Description 8) and 3,4-dibromoaniline (169 mg). Purified using the Biotage Horizon system to afford the title compound as a white solid (76 mg).

NMR (DMSO-d6) δ 1.09-1.23 (8H, d, m), 1.60-1.63 (2H, d), 1.76 (1H, m), 3.10 (2H, t), 3.28 (2H, t), 3.41 (1H, m), 3.85 (2H, d), 6.78 (1H, s), 7.48 (1H, d), 7.59 (1H, d), 8.15 (1H, s), 8.38 (2H, t), 9.52 (1H, s).

LC/MS t=3.5 min, [MH⁺] 510, consistent with molecular formula C₂₁H₂₅⁷⁹Br₂N₃O₂

### EXAMPLE 297

### 6-(4-Bromo-3-fluoro-phenylamino)-4-isopropyl-N-(tetrahydropyran-4-ylmethyl)-nicotinamide

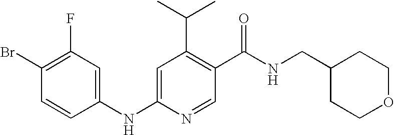

Prepared in a manner similar to Example 294 from 6-chloro-4-isopropyl-N-(tetrahydropyran-4-ylmethyl)-nicotinamide (Description 8) and 4-bromo-3-fluoro-aniline (128 mg). Purified by trituration with ether to afford the title compound as a white solid (88 mg).

NMR (DMSO-d6) δ 1.15-1.25 (8H, d, m), 1.60-1.63 (2H, d), 1.76 (1H, m), 3.10 (2H, t), 3.28 (2H, t), 3.41 (1H, m), 3.85 (2H, d), 6.81 (1H, s), 7.30 (1H, d), 7.54 (1H, t), 8.04 (1H, d), 8.15 (1H, s), 8.40 (1H, t), 9.64 (1H, s).

LC/MS t=3.3 min, [MH⁺] 450, consistent with molecular formula C₂₁H₂₅F⁷⁹BrN₃O₂

### EXAMPLE 298

### 6-(2-Chloro-4-trifluoromethyl-phenylamino)-N-cyclopentylmethyl-4-isopropyl-nicotinamide

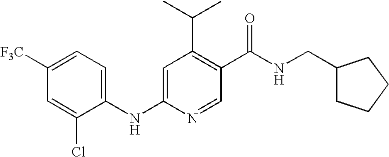

Prepared in a manner similar to Example 294 from 6-chloro-N-cyclopentylmethyl-4-isopropyl-nicotinamide and 2-chloro-4-trifluoromethylaniline, to give the title compound (30 mg).

NMR (DMSO-d6) δ 1.18 (8H, m), 1.50-1.68 (6H, m), 2.11 (1H, m), 3.16 (2H, s), 3.37 (1H, s), 7.29 (1H, s), 7.64 (1H, d), 7.83 (1H, s), 8.09 (1H, s), 8.43 (1H, s), 8.52 (1H, d), 8.80 (1H, s).

LC/MS t=4.0 min, [MH⁺] 440, consistent with molecular formula C₂₂H₂₅³⁵ClF₃N₃O

### EXAMPLE 299

### 6-(3,4-Difluoro-phenylamino)-4-isopropyl-N-(tetrahydropyran-4-ylmethyl)-nicotinamide

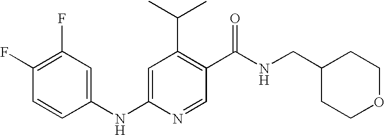

A mixture of 6-chloro-4-isopropyl-N-(tetrahydropyran-4-ylmethyl)-nicotinamide (Description 8) (100 mg), 3,4-difluoroaniline (ex Lancaster, 87 mg), methanesulfonic acid (44 μl) in 1,4-dioxane (1 ml) was irradiated under microwave conditions at 180° for 30 minutes. The solid was dissolved in methanol then evaporated under reduced pressure. The mixture was partitioned between ethyl acetate (5 ml) and brine (2 ml) whereby a solid remained at the interface. The solid was filtered off and washed with water and ethyl acetate to afford the title compound (43 mg).

NMR (DMSO-d6) δ 1.16-1.25 (8H, d, m), 1.60-1.62 (2H, d), 1.75 (1H, m), 3.10 (2H, t), 3.28 (2H, t), 3.41 (1H, m), 3.85 (2H, d), 6.85 (1H, s), 7.29 (1H, d), 7.37 (1H, q), 7.97 (1H, s), 8.08 (1H, s), 8.45 (1H, t), 9.80 (1H, s).

LC/MS t=3.0 min, [MH⁺] 390, consistent with molecular formula C₂₁H₂₅F₂N₃O₂

### EXAMPLE 300

### 6-(4-Chloro-3-trifluoromethyl-phenylamino)-4-isopropyl-N-(tetrahydropyran-4-ylmethyl)-nicotinamide

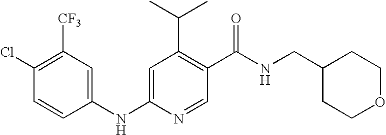

Prepared in a manner similar to Example 291 from 6-chloro-4-isopropyl-N-(tetrahydropyran-4-ylmethyl)-nicotinamide (Description 8) (100 mg) and 4-chloro-3-trifluoromethyl-aniline (ex Lancaster, 131 mg). Purified by trituration with ether to afford the title compound as a white solid (79 mg).

NMR (DMSO-d6) δ1.16-1.24 (8H, d, m), 1.60-1.63 (2H, d), 1.76 (1H, m), 3.11 (2H, t), 3.28 (2H, t), 3.41 (1H, m), 3.85 (2H, d), 6.80 (1H, s), 7.58 (1H, d), 7.91 (1H, d), 8.16 (1H, s), 8.39 (1H, s), 8.41 (1H, t), 9.70 (1H, s).

LC/MS t=3.5 min, [MH⁺] 456, consistent with molecular formula C₂₂H₂₅³⁵ClF₃N₃O₂

### EXAMPLE 301

### 6-(4-Methyl-3-trifluoromethyl-phenylamino)-4-isopropyl-N-(tetrahydropyran-4-ylmethyl)-nicotinamide

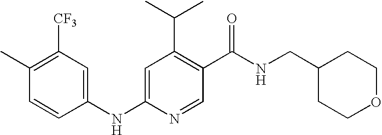

Prepared in a manner similar to Example 291 from 6-chloro-4-isopropyl-N-(tetrahydropyran-4-ylmethyl)-nicotinamide (Description 8) (100 mg) and 4-methyl-3-trifluoromethylaniline (ex Lancaster, 18 mg). Purified by trituration with ether to afford the title compound as a white solid (105 mg).

NMR (DMSO-d6) δ1.15-1.24 (8H, d, m), 1.60-1.63 (2H, d), 1.76 (1H, m), 2.36 (3H, s), 3.11 (2H, t), 3.28 (2H, t), 3.41 (1H, m), 3.85 (2H, d), 6.76 (1H, s), 7.31 (1H, d), 7.76 (1H, d), 8.13 (1H, s), 8.18 (1H, s), 8.37 (1H, t), 9.45 (1H, s).

LC/MS t=3.2 min, [MH⁺] 436, consistent with molecular formula C₂₃H₂₈F₃N₃O₂

### EXAMPLE 302

### 6-(2-Chloro-4-trifluoromethoxy-phenylamino)-4-isopropyl-N-(tetrahydropyran-4-ylmethyl)-nicotinamide

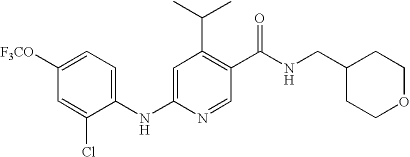

Prepared in a manner similar to Example 291 from 6-chloro-4-isopropyl-N-(tetrahydropyran-4-ylmethyl)-nicotinamide (Description 8) (100 mg) and 2-chloro-4-trifluoromethoxyaniline (ex Acros, 142 mg). Purified using the Biotage Horizon system detailed at the beginning of the experimental section and by trituration with ether to afford the title compound as a white solid (20 mg).

NMR (DMSO-d6) δ 1.16-1.23 (8H, d, m), 1.59-1.62 (2H, d), 1.75 (1H, m), 3.11 (2H, t), 3.28 (2H, t), 3.37 (1H, m), 3.84 (2H, d), 7.09 (1H, s), 7.34 (1H, d), 7.58 (1H, s), 8.04 (1H, s), 8.20 (1H, d), 8.38 (1H, t), 8.66 (1H, s).

LC/MS t=3.4 min, [MH⁺] 472, consistent with molecular formula C₂₂H₂₅³⁵ClF₃N₃O₃

### EXAMPLE 303

### 6-(2-Cyano-3-methyl-phenylamino)-4-isopropyl-N-(tetrahydropyran-4-ylmethyl)-nicotinamide

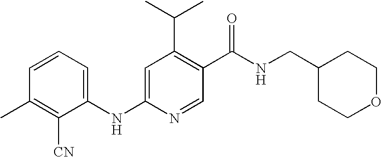

Prepared in a manner similar to Example 221 from 6-chloro-4-isopropyl-N-(tetrahydropyran-4-ylmethyl)-nicotinamide (Description 8) (100 mg) and 2-cyano-3-methylaniline (ex Fluka, 44 mg) to give the title compound (60 mg).

NMR (DMSO-d6) δ1.16-1.23 (8H, d, m), 1.59-1.62 (2H, d), 1.75 (1H, m), 2.46 (3H, s), 3.11 (2H, t), 3.28 (2H, t), 3.37 (1H, m), 3.84 (2H, d), 6.96 (1H, s), 7.07 (1H, d), 7.48 (1H, t), 7.67 (1H, d), 8.03 (1H, s), 8.39 (1H, t), 9.13 (1H, s).

LC/MS t=2.7 min, [MH⁺] 393, consistent with molecular formula C₂₃H₂₈N₄O₂

### EXAMPLE 304

### 6-(3-Chloro-2-cyano-phenylamino)-4-isopropyl-N-(tetrahydropyran-4-ylmethyl)-nicotinamide

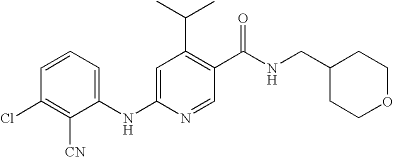

Prepared in a manner similar to Example 221 from 6-chloro-4-isopropyl-N-(tetrahydropyran-4-ylmethyl)-nicotinamide (Description 8) (100 mg) and 3-chloro-2-cyanoaniline (ex Lancaster, 51 mg) to give the title compound (64 mg).

NMR (DMSO-d6) δ 1.17-1.23 (8H, d, m), 1.59-1.62 (2H, d), 1.75 (1H, m), 3.11 (2H, t), 3.28 (2H, t), 3.37 (1H, m), 3.85 (2H, d), 7.03 (1H, s), 7.32 (1H, d), 7.60 (1H, t), 7.87 (1H, d), 8.07 (1H, s), 8.42 (1H, t), 9.41 (1H, s).

LC/MS t=2.8 min, [MH⁺] 413, consistent with molecular formula C₂₂H₂₅³⁵ClN₄O₂

### EXAMPLE 305

### 6-(3-Chloro-phenylamino)-4-(1-hydroxy-methyl-ethyl)-N-(tetrahydropyran-4-ylmethyl)-nicotinamide

### 1) 6-Chloro-1,1,dimethyl-1H-furo[3,4-c]pyridin-3-one

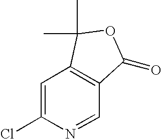

To a solution of 2,2,6,6,-tetramethylpiperidine (ex Aldrich, 13.44 g) in tetrahydrofuran (90 ml) at −55° C. under nitrogen was added dropwise 1.6M butyl lithium in hexane (ex Aldrich, 80 ml). After 30 minutes a solution of 6-chloronicotinic acid (ex Aldrich, 5 g) in tetrahydrofuran (40 ml) was added dropwise and the solution stirred at −71° C. for 2 hours. The solution was treated with acetone (23 ml) and then allowed to warm to room temperature. The solvent was removed under reduced pressure and the residue dissolved in water (100 ml) and acidified to pH 3 with concentrated hydrochloric acid. The precipitated white solid was filtered off washed with water and dried to afford the title compound (4.42 g).

NMR (DMSO-d6) δ1.65 (6H, s), 8.11 (1H, s), 8.91 (1H, s)

LC/MS t=2.0 min, [MH⁺] 198, consistent with molecular formula C₉H₈³⁵ClNO₂

### 2) 6-(3-Chloro-phenylamino)-1,1,dimethyl-1H-furo[3,4-c]pyridin-3-one

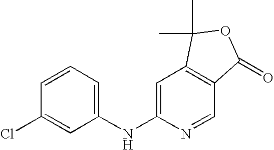

A mixture of 6-Chloro-1,1,dimethyl-1H-furo[3,4-c]pyridin-3-one (100 mg), 3-chloroaniline (ex Lancaster, 318 mg), methanesulfonic acid (65 μl) in 1,4-dioxane (1 ml) was irradiated under microwave conditions at 180° for 30 minutes. The solid was dissolved in methanol then evaporated under reduced pressure and the residue partitioned between ethyl acetate (5 ml) and water (2 ml) and the aqueous layer separated. The organic layer was dried over anhydrous magnesium sulphate, filtered and evaporated under reduced pressure. Purified by trituration with ether to afford the title compound as a white solid (30 mg).

NMR (DMSO-d6) δ 1.61 (6H, s), 6.91 (1H, s), 7.04 (1H, d), 7.34 (1H, t), 7.55 (1H, d), 7.93 (1H, t), 8.69 (1H, s), 9.96 (1H, s).

LC/MS t=3.3 min, [MH⁺] 289, consistent with molecular formula C₁₅H₁₃³⁵ClN₂O₂

### 3) 6-(3-Chloro-phenylamino)-4-(1-hydroxy-methyl-ethyl)-N-(tetrahydropyran-4-ylmethyl)-nicotinamide

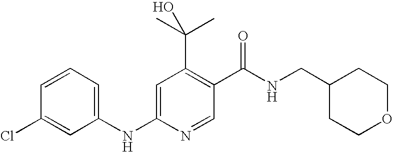

To a solution of 4-aminomethyltetrahydropyran (ex Combi-Blocks, Inc, 60 mg) in dry dichloromethane (2 ml) under nitrogen, was added dropwise 2.0M trimethylaluminium in hexane (ex Aldrich, 280 μl) and the solution stirred for 15 minutes. Then a solution of 6-(3-Chloro-phenylamino)-1,1,dimethyl-1H-furo[3,4-c]pyridin-3-one (70 mg) in dry dichloromethane (2 ml) was added and the mixture stirred at 40° C. overnight. A further portion of 4-aminomethyltetrahydropyran (80 mg) and 2.0M trimethylaluminium in hexane (380 μl) in dry dichloromethane (3 ml) was added and the mixture stirred for 48 h. The solvent was evaporated under reduced pressure and the residue partitioned between ethyl acetate (10 ml) and water (5 ml) and the aqueous layer separated. The organic layer was dried over anhydrous magnesium sulphate, filtered and evaporated under reduced pressure. Purified using the Biotage Horizon system detailed at the beginning of the experimental section to afford the title compound as a white solid (40 mg).

NMR (DMSO-d6) δ1.18-1.23 (2H, m), 1.47 (6H, s), 1.62-1.65 (2H, d), 1.80 (1H, m), 3.11 (2H, t), 3.28 (2H, t), 3.85 (2H, d), 6.06 (1H, s), 6.93 (1H, d), 7.05 (1H, s), 7.28 (1H, t), 7.48 (1H, d), 8.07 (1H, s), 8.17 (1H, s), 8.67 (1H, t), 9.53 (1H, s).

LC/MS t=3.0 min, [MH⁺] 404, consistent with molecular formula C₂₁H₂₆³⁵ClN₃O₃

### EXAMPLE 306

The compound below was prepared as for Example 75 from the intermediate of Description 15.

| | | | | RT (min), (MH+) |
|---|---|---|---|---|
| | | | Purification | Consistent with |
| Name | Structure | Method | Method | molecular formula |
| 4-tert-Butyl-6-(3,4- dichloro- phenylamino)-N- (tetrahydro-pyran-4- ylmethyl)- nicotinamide | 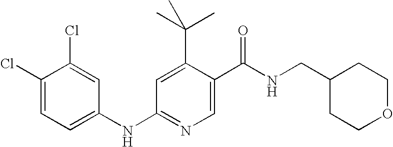 | G | E | 3.6 436 C₂₂H₂₇³⁵Cl₂N₃O₂ |

Formulations for pharmaceutical use incorporating compounds of the present invention can be prepared in various forms and with numerous excipients. Examples of such formulations are given below.

### EXAMPLE 307

### Inhalant Formulation

A compound of formula (I) or a pharmaceutically acceptable derivative thereof, (1 mg to 100 mg) is aerosolized from a metered dose inhaler to deliver the desired amount of drug per use.

### EXAMPLE 308

### Tablet Formulation

| Tablets/Ingredients | | Per Tablet |
|---|---|---|
| 1. | Active ingredient | 40 mg |
| | (Compound of formula (I) or pharmaceutically | |
| | acceptable derivative) | |
| 2. | Corn Starch | 20 mg |
| 3. | Alginic acid | 20 mg |
| 4. | Sodium Alginate | 20 mg |
| 5. | Mg stearate | 1.3 mg |

Procedure for tablet formulation:

Ingredients 1, 2, 3 and 4 are blended in a suitable mixer/blender. Sufficient water is added portion-wise to the blend with careful mixing after each addition until the mass is of a consistency to permit its conversion to wet granules. The wet mass is converted to granules by passing it through an oscillating granulator using a No. 8 mesh (2.38 mm) screen. The wet granules are then dried in an oven at 140° F. (60° C.) until dry. The dry granules are lubricated with ingredient No. 5, and the lubricated granules are compressed on a suitable tablet press.

### EXAMPLE 309

### Parenteral Formulation

A pharmaceutical composition for parenteral administration is prepared by dissolving an appropriate amount of a compound of formula (I) in polyethylene glycol with heating. This solution is then diluted with water for injections Ph Eur. (to 100 ml). The solution is then rendered sterile by filtration through a 0.22 micron membrane filter and sealed in sterile containers.

It is to be understood that the present invention covers all combinations of particular and preferred groups described herein above

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

1. A compound of formula (I): 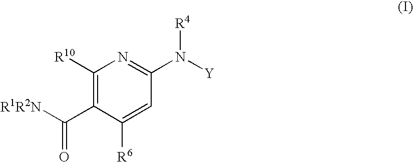
wherein:
Y is phenyl, unsubstituted or substituted with one, two or three substituents selected from C₁₋₆ alkyl, halosubstitutedC₁₋₆ alkyl, C₁₋₆ alkoxy, a hydroxy group, a cyano group, halo, a C₁₋₆alklsulfonyl group, —CONH₂, —NHCOCH₃—COOH halosubstitutedC₁₋₆ alkoxy, SO₂NR^{8a}R^{8b} and C₁₋₆ alkynyl;
R¹ is selected from hydrogen, C₁₋₆ alkyl, C₃₋₆ cycloalkyl, and halosubstitutedC₁₋₆ alkyl;
R² is (CH₂)ₘR³ where m is 0 or 1;
or R¹ and R² together with N to which they are attached form 4- to 8-membered non-aromatic heterocyclyl ring optionally substituted with 1, 2 or 3 substituents selected from: C₁₋₆ alkyl, C₁₋₆ alkoxy, a hydroxy group, a cyano group, halo, a sulfonyl group, methylsulfonyl, NR^{8a}R^{8b}, CH₂phenyl. NHCOCH₃ (═O), CONHCH₃ or NHSO₂CH₃;
R³ is a 4- to 8-membered non-aromatic heterocyclyl group, a C₃₋₈ cycloalkyl group, a straight or branched C₁₋₁₀ alkyl, a C₂₋₁₀alkenyl, a C₃₋₈cycloalkenyl, a C₂₋₁₀alkynyl, or a C₃₋₈cycloalkynyl any of which can be unsubstituted or substituted with C₁₋₆ alkyl, C₁₋₆ alkoxy, a hydroxy group, a cyano group, halo, a sulfonyl group, methylsulfonyl, NR^{8a}R^{8b}, CH₂phenyl, NHCOCH₃, (═O), CONHCH₃ or NHSO₂CH₃ or R³ can be R⁵;
R⁴ is selected from hydrogen, C₁₋₆ alkyl, C₃₋₆ cycloalkyl, or halosubstitutedC₁₋₆ alkyl, COCH₃, and SO₂Me;
R⁵ is 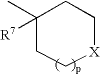
wherein p is 0, 1 or 2, and X is CH₂, O, or S;
R⁶ is a substituted or unsubstituted (C₁₋₆)alkyl or chloro and R¹⁰ is hydrogen or R¹⁰ is a substituted or unsubstituted (C₁₋₆)alkyl or chloro and R⁶ is hydrogen wherein said substituted (C₁₋₆)alkyl group is substituted with 1, 2 or 3 substitutents selected from hydroxy. C₁₋₆alkyoxy, cyano, halo, NR^{8a}R^{8b}, CONR^{8a}R^{8b}, SO₂NR^{8a}R^{8b}, NR^{8a}COR^{8b} and NR^{8a}SO₂R^{8b};
R⁷ is OH, C₁₋₆alkoxy, NR^{8a}R^{8b}, NHCOR⁹, NHSO₂R⁹ or SOqR⁹;
R^{8a} is H or C₁₋₆alkyl;
R^{8b} is H or C₁₋₆alkyl;
R⁹ is C₁₋₆alkyl;
q is 0, 1 or 2;
or a pharmaceutically acceptable derivative thereof.

2. A compound as of formula (Ia): 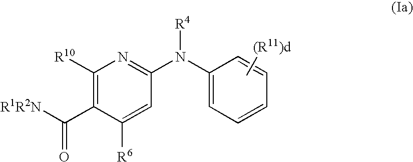
R¹ is selected from hydrogen, C₁₋₆ alkyl, C₃₋₆ cycloalkyl, and halosubstitutedC₁₋₆ alkyl;
R² is (CH₂)ₘR³ where m is 0 or 1;
or R¹ and R² together with N to which they are attached form a non-aromatic heterocyclyl ring selected from azetidinyl, pyrrolidinyl, morpholinyl, piperazinyl, piperidinyl, tetrahydropyridinyl, azapine, oxapine, azacyclooctanyl, azaoxacyclooctanyl and azathiacyclooctanyl, any of which can be unsubstituted or substituted with 1, 2 or 3 substituents selected from; C₁₋₆ alkyl, C₁₋₆ alkoxy, hydroxy, cyano, halo, sulfonyl, methylsulfonyl, NR^{8a}R^{8b}, CH₂phenyl, NHCOCH₃, (═O), CONHCH₃ and NHSO₂CH₃;
R³ is 2- or 3-azetidinyl, oxetanyl, thioxetanyl, thioxetanyl-s-oxide, thioxetanyl-s,s-dioxide, dioxalanyl, pyrrolidinyl, tetrahydrofuranyl, tetrahydrothiophenyl, tetrahydrothiophenyl-s,s-dioxide, morpholinyl, piperidinyl, piperazinyl, tetrahydropyranyl, tetrahydrothiopyranyl, thiomorpholinyl, thiomorpholinyl-s,s-dioxide, tetrahydropyridinyl, dioxanyl, tetrahydro-thiopyran 1,1 dioxide, azapine, oxapine, azacyclooctanyl, azaoxacyclooctanyl, azathiacyclooctanyl, oxacylcooctanyl, thiacyclooctanyl, a C₃₋₈ cycloalkyl group, a straight or branched C₁₋₁₀ alkyl, a C₂₋₁₀alkenyl, a C₃₋₈cycloalkenyl, a C₂₋₁₀alkynyl, or a C₃₋₈cycloalkynyl or R⁵; any of which can be unsubstituted or substituted with 1, 2 or 3 substituents selected from C₁₋₆ alkyl, C₁₋₆ alkoxy, hydroxy, cyano, halo, sulfonyl, methylsulfonyl, NR^{8a}R^{8b}, CH₂phenyl, NHCOCH₃, (═O), CONHCH₃ and NHSO₂CH₃;
R⁴ is selected from hydrogen, C₁₋₆ alkyl, C₃₋₆ cycloalkyl, or halosubstitutedC₁₋₆ alkyl, COCH₃, and SO₂Me;
R⁵ is 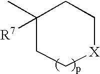
wherein p is 0, 1 or 2, and X is CH₂, O or S;
R⁵ is a substituted or unsubstituted (C₁₋₆)alkyl or chloro and R¹⁰ is hydrogen or R¹⁰ is a substituted or unsubstituted (C₁₋₆)alkyl or chloro and R⁶ is hydrogen wherein said substituted (C₁₋₆)alkyl group is substituted with 1, 2 or 3 substitutents selected from hydroxy, C₁₋₆alkyoxy, cyano, halo, NR^{8a}R^{8b}, CONR^{8a}R^{8b}, SO₂NR^{8a}R^{8b}, NR^{8a}COR^{8b} and NR^{8a}SO₂R^{8b};
R⁷ is OH, C₁₋₆alkoxy, NR^{8a}R^{8b}, NHCOR⁹, NHSO₂R⁹ or SOqR⁹;
R^{8a} is H or C₁₋₆alkyl;
R^{8b} is H or C₁₋₆alkyl;
R⁹ is C₁₋₆alkyl;
R¹¹ is C₁₋₆ alkyl, halosubstitutedC₁₋₆ alkyl, C₁₋₆ alkoxy, hydroxy, cyano, halo, C₁₋₆alkylsulfonyl group, —CONH₂, —NHCOCH₃, —COOH, halosubstituted C₁₋₆ alkoxy SO₂NR^{8a}R^{8b} or C₁₋₆ alkynyl;
q is 0, 1 or 2;
d is 0, 1, 2, or 3;
or a pharmaceutically acceptable derivative thereof.

3. A compound as claimed in claim 1 or wherein R¹ is hydrogen.

4. A compound as claimed in claim 1 wherein R⁴ is C₁₋₆ alkyl or hydrogen.

5. A compound as claimed in claim 1 wherein R⁶ is t-butyl, isopropyl or CF₃.

6. A pharmaceutical composition comprising a compound as claimed in claim 1.

7. A pharmaceutical composition as claimed in claim 6 further comprising a pharmaceutical carrier or diluent thereof.

8. A method of treating a mammal suffering from a condition which is mediated by the activity of cannabinoid 2 receptors which comprises administering to said mammal a therapeutically effective amount of a compound as claimed in claim 1.

9. A method of treatment as claimed in claim 8 wherein the condition is an immune disorder, an inflammatory disorder, pain, rheumatoid arthritis, multiple sclerosis, osteoarthritis or osteoporosis.

10. The method as claimed in claim 9, wherein said pain is selected from inflammatory pain, viseral pain, cancer pain, neuropathic pain, lower back pain, muscular sceletal, post operative pain, acute pain and migraine.

11. The method as claimed in claim 8, wherein said mammal is a human.

12. A compound selected from
6-(3-Chloro-phenyl-amino)-4-isopropyl-N-(tetrahydro-pyran-4-ylmethyl)-nicotinamide;
6-(3-Bromo-phenyl-amino)-4-isopropyl-N-(tetrahydro-pyran-4-ylmethyl)-nicotinamide;
6-(2,4-Dichloro-phenylamino)-4-isopropyl-N-(tetrahydro-pyran-4-ylmethyl)-nicotinamide;
4-isopropyl-N-(tetrahydro-pyran-4-ylmethyl)-6-(3-trifluoromethoxy-phenylamino)-nicotinamide;
4-tert-Butyl-6-(2,4-di-chloro-phenylamino)-N-(tetrahydro-pyran-4-ylmethyl)-nicotinamide;
6-(3-Chloro-4-cyano-phenylamino)-4-isopropyl-N-(tetrahydro-pyran-4-ylmethyl)-nicotinamide;
6-(2-Fluoro-3-trifluoromethyl-phenylamino)-4-isopropyl-N-(tetrahydro-pyran-4-ylmethyl)-nicotinamide;
6-(4-Bromo-2-chloro-phenylamino)-4-isopropyl-N-(tetrahydro-pyran-4-ylmethyl)-nicotinamide;
6-(3,4-Dichloro-phenylamino)-4-isopropyl-N-(tetrahydro-pyran-4-ylmethyl)-nicotinamide;
6-(2-Bromo-4-trifluoromethoxy-phenylamino)-4-isopropyl-N-(tetrahydro-pyran-4-ylmethyl)-nicotinamide;
6-(3,5-Difluoro-phenylamino)-4-isopropyl-N-tetrahydro-pyran-4-ylmethyl)-nicotinamide;
6-(2,4-Dichloro-phenylamino)-N-(tetrahydro-pyran-4-ylmethyl)-4-trifluoromethyl-nicotinamide;
and pharmaceutically acceptable derivatives thereof.

13. A pharmaceutical composition comprising a compound as claimed in claim 12.

14. A method of treating a mammal suffering from a condition which is mediated by the activity of cannabinoid 2 receptors which comprises administering to said mammal a therapeutically effective amount of a compound as claimed in claim 12.

15. A method of treatment as claimed in claim 14 wherein the condition is an immune disorder, an inflammatory disorder, pain, rheumatoid arthritis, multiple sclerosis, osteoarthritis or osteoporosis.

16. The method as claimed in claim 15, wherein said pain is selected from inflammatory pain, viseral pain, cancer pain, neuropathic pain, lower back pain, muscular sceletal, post operative pain, acute pain and migraine.

17. The method as claimed in claim 14, wherein said mammal is a human.
